SEQUENCE LISTING (24 sequences)

SEQ ID NO: 1 - protein, 345 aa, Homo sapiens
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 2 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 3 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 4 - protein, 5 aa, Artificial Sequence
  GC5B596 HCDR1
GYTMN

SEQ ID NO: 5 - protein, 17 aa, Artificial Sequence
  GC5B596 HCDR2
LINPYNSDTNYAQKLQG

SEQ ID NO: 6 - protein, 9 aa, Artificial Sequence
  GC5B596 HCDR3
VALRVALDY

SEQ ID NO: 7 - protein, 11 aa, Artificial Sequence
  GC5B596 LCDR1
KASQNVATHVG

SEQ ID NO: 8 - protein, 7 aa, Artificial Sequence
  GC5B596 LCDR2
SASYRYS

SEQ ID NO: 9 - protein, 9 aa, Artificial Sequence
  GC5B596 LCDR3
QQYNRYPYT

SEQ ID NO: 10 - protein, 118 aa, Artificial Sequence
  GC5B596 VH
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 11 - protein, 107 aa, Artificial Sequence
  GC5B596 VL
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 12 - protein, 445 aa, Artificial Sequence
  GC5B596 HC
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 210 aa, Artificial Sequence
  GC5B596 LC
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 14 - protein, 5 aa, Artificial Sequence
  CD3B219 HCDR1
TYAMN

SEQ ID NO: 15 - protein, 19 aa, Artificial Sequence
  CD3B219 HCDR2
RIRSKYNNYATYYAASVKG

SEQ ID NO: 16 - protein, 14 aa, Artificial Sequence
  CD3B219 HCDR3
HGNFGNSYVSWFAY

SEQ ID NO: 17 - protein, 14 aa, Artificial Sequence
  CD3B219 LCDR1
RSSTGAVTTSNYAN

SEQ ID NO: 18 - protein, 7 aa, Artificial Sequence
  CD3B219 LCDR2
GTNKRAP

SEQ ID NO: 19 - protein, 9 aa, Artificial Sequence
  CD3B219 LCDR3
ALWYSNLWV

SEQ ID NO: 20 - protein, 125 aa, Artificial Sequence
  CD3B219 VH
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 21 - protein, 112 aa, Artificial Sequence
  CD3B219 VL
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 22 - protein, 452 aa, Artificial Sequence
  CD3B219 HC
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 23 - protein, 215 aa, Artificial Sequence
  CD3B219 LC
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 24 - protein, 327 aa, Artificial Sequence
  Wild-type IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2022/0177584 A1
(43) Pub. Date: Jun. 9, 2022

(54) METHODS FOR TREATING MULTIPLE MYELOMA

(71) Applicant: Janssen Pharmaceutica NV, Beerse (BE)

(72) Inventors: Suzette Girgis, Spring House, PA (US); Jenna Goldberg, Raritan, NJ (US); Brandi Hilder, Spring House, PA (US); Xuewen Ma, Spring House, PA (US); Jeffery Russell, Salt Lake City, UT (US); Raluca Verona, Spring House, PA (US); Shiyi Yang, Spring House, PA (US); Kodandaram Pillarisetti, King of Prussia, PA (US)

(21) Appl. No.: 17/477,435

(22) Filed: Sep. 16, 2021

Related U.S. Application Data
(60) Provisional application No. 63/187,888, filed on May 12, 2021.
(60) Provisional application No. 63/116,549, filed on Nov. 20, 2020.
(60) Provisional application No. 63/079,294, filed on Sep. 16, 2020.

(51) Int. Cl. C07K 16/28; A61K 39/395; A61P 35/00

(52) U.S. Cl. CPC C07K 16/2809; C07K 16/28; A61K 39/3955; A61K 2039/54; C07K 2317/31; C07K 2317/565; C07K 2317/56; A61P 35/00

(57) ABSTRACT

Methods of treating a hematological malignancy using a GPRC5D×CD3 bispecific antibody are described. The hematological malignancy can be a relapsed or refractory multiple myeloma, and the GPRC5D×CD3 bispecific antibody can be talquetamab.

### CROSS-REFERENCE TO RELATED APPLICATIONS

This application claims the benefit of U.S. Provisional Application Ser. No. 63/079,294, filed 16 Sep. 2020, U.S. Provisional Application Ser. No. 63/116,549, filed 20 Nov. 2020, and U.S. Provisional Application Ser. No. 63/187,888, filed 12 May 2021. The entire content of the aforementioned applications is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 3, 2021, is named “Sequence Listing 065768_89US4txt” and is 26.8 kilobytes in size.

### FIELD OF THE INVENTION

Methods of treating a hematological malignancy, particularly relapsed or refractory multiple myeloma, using a GPRC5D×CD3 bispecific antibody are disclosed.

### BACKGROUND OF THE INVENTION

G-protein coupled receptor, class C, group 5, member D (GPRC5D) is an orphan, atypical, class C GPCR first identified in 2001 (Brauner-Osborne et al., Biochim Biophys Acta., 1518(3):237-248, 2001). GPRC5D and other group 5 GPCRs have unusually short amino-terminal domains for class C receptors, and are therefore, predicted to be conformationally similar to class A receptors. In this regard they are unique, with sequence homology to class C GPCRs and predicted structural topology comparable to class A receptors. Functional consequence of GPRC5D activation has not been described and the ligand remains unknown. The gene has three exons and is located on chromosome 12p13.3 in humans. GPRC5D receptor is highly conserved among various species and shares 92% identity with cynomolgus monkey GPRC5D.

GPRC5D mRNA is predominantly expressed in all malignant plasma cells from MM patients (Atamaniuk J A et al. Eur J Clin Invest, 42(9) 953-960; 2012; Frigyesi-blood and Cohen, et al., Hematology 18(6): 348-35; 2013). GPRC5D expression is variable among the patients and correlates well with plasma cell burden and genetic aberrations such as Rb-1 deletion (Atamaniuk J A et al., Eur J Clin Invest, 42(9) 953-960; 2012).

Multiple myeloma (MM) is the second most common hematological malignancy and constitutes 2% of all cancer deaths. MM is a heterogeneous disease and caused mostly by chromosome translocations inter alia t(11;14), t(4; 14), t(8;14), del(13), del(17) (Drach et al., Blood. 1998; 92(3):802-809, Gertz et al., Blood. 2005; 106(8). 2837-2840; Facon et al., Blood. 2001; 97(6): 1566-1571). MM-affected patients can experience a variety of disease-related symptoms due to, bone marrow infiltration, bone destruction, renal failure, immunodeficiency, and the psychosocial burden of a cancer diagnosis. Based on people diagnosed with MM between 2009 and 2015, the 5-year relative survival rate for MM was approximately 51%. This highlights that MM is a difficult-to-treat disease where there are currently insufficient curative options.

Relapsed and refractory MM constitutes a specific unmet medical need. Patients with relapsed and refractory disease are defined as those who achieve minor response or better then progress while on therapy or who experience progression within 60 days of their last therapy. Patients who progress after receiving both an immunomodulatory drug and proteasome inhibitor have limited options. Heavily pretreated patients often present with a compromised immune system, which can result in other disease conditions such as opportunistic infections and toxicities (e.g., myelosuppression, peripheral neuropathy, deep vein thrombosis) that persist from prior treatment. Furthermore, patients with advanced MM are often elderly and are susceptible to serious treatment-emergent adverse events (TEAEs) with continued exposure to these therapies. After standard available therapies (such as proteasome inhibitors, immunomodulatory drugs, and monoclonal antibodies) have been exhausted, there is no standard therapy. Selinexor, BLENREP (belantamab mafodotin-blmf), recently approved Melfufen (melphalan flufenamide) administered in combination with dexamethasone, as well as recently approved Ide-cel (idecabtagene viceleucel, formerly termed bb2121) are licensed in the United States for this highly refractory disease setting. The remaining options for these patients are either entry into a clinical trial, or they can be offered retreatment with a prior treatment regimen (if the toxicity profile for retreatment permits). But often, if no other treatment options remain, they are provided with palliative care to ameliorate disease-related symptoms only. In elderly population, for whom stem cell transplantation is often not a viable option, and in patients with refractory disease who have exhausted all available therapies, the median overall survival is only 8 to 9 months (Kumar et al., Leukemia, 2012, 26:149-157; Usmani et al., Oncolgist, 2016, 21:1355-1361). For patients with disease that is refractory to commonly administered proteasome inhibitors and immunomodulatory drugs, the medium overall survival decreases to only 5 months (Usmani et al., 2016).

Thus, there remains an unmet medical need to develop treatment options for MM patients, particularly those who are relapsed or refractory to treatment with a prior anti-cancer therapeutic.

### SUMMARY OF THE INVENTION

In one general aspect, provided herein is a method of treating a hematological malignancy, such as multiple myeloma, in a subject in need thereof, comprising administering to the subject a therapeutically effective amount of a GPRC5D×CD3 bispecific antibody or antigen binding fragment thereof, wherein the subject is relapsed or refractory to treatment with a prior anti-cancer therapy.

In one embodiment of a method of the application, the GPRC5D×CD3 bispecific antibody or antigen binding fragment thereof comprises a GPRC5D binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

In a further embodiment of a method of the application, the GPRC5D binding domain comprises a heavy chain variable region (VH) having the amino acid sequence of SEQ ID NO: 10 and a light chain variable region (VL) having the amino acid sequence of SEQ ID NO: 11, and the CD3 biding domain comprises a VH having the amino acid sequence of SEQ ID NO: 20 and a VL having the amino acid sequence of SEQ ID NO: 21.

In a yet further embodiment of a method of the application, the GPRC5D×CD3 bispecific antibody is of IgG4 isotype and comprises phenylalanine at position 405 and arginine at position 409 in a first heavy chain (HC1) and leucine at position 405 and lysine at position 409 in a second heavy chain (HC2).

In a yet further embodiment of a method of the application, the GPRC5D×CD3 bispecific antibody further comprises proline at position 228, alanine at position 234 and alanine at position 235 in both the HC1 and the HC2.

In a yet further embodiment of a method of the application, the GPRC5D×CD3 bispecific antibody comprises the HC1 having the amino acid sequence of SEQ ID NO: 12, a first light chain (LC1) having the amino acid sequence of SEQ ID NO: 13, the HC2 having the amino acid sequence of SEQ ID NO: 22 and a second light chain (LC2) having the amino acid sequence of SEQ ID NO: 23.

In a yet further embodiment of a method of the application, the GPRC5D×CD3 bispecific antibody is talquetamab.

In a yet further embodiment of a method of the application, the GPRC5D×CD3 bispecific antibody is administered intravenously or subcutaneously at a dose of about 0.2 μg/kg to about 1200 μg/kg.

In a yet further embodiment of a method of the application, the GPRC5D×CD3 bispecific antibody is administered intravenously at a dose of about 0.2 μg/kg to about 500 μg/kg, preferably about 1 μg/kg to about 300 μg/kg, most preferably about 10 μg/kg to about 200 μg/kg, such as about 10, 20, 30, 40, 50, 60, 70, 80, 90, 100, 110, 120, 130, 140, 150, 160, 170, 180, 190, 200 μg/kg, or any value in between. The dose can be administered monthly, tri-weekly (i.e., one dose every three weeks), bi-weekly (i.e., one dose every other week), weekly, twice weekly (i.e., two doses every week).

In a yet further embodiment of a method of the application, the GPRC5D×CD3 bispecific antibody is administered subcutaneously at a dose of about 0.5 μg/kg to about 2400 μg/kg, about 0.5 μg/kg to about 1200 μg/kg, or about 1 μg/kg to about 100 μg/kg, or about 10 μg/kg to about 800 μg/kg, such as about 10, 20, 30, 40, 50, 60, 70, 80, 90, 100, 135, 150, 200, 250, 300, 350, 400, 405, 450, 500, 550, 600, 650, 700, 750, 800, 900, 950, 1000, 1050, 1100, 1150, 1200 μg/kg, or any value in between. The dose can be administered monthly, tri-weekly, bi-weekly, weekly, or twice weekly. In certain embodiments, the GPRC5D×CD3 bispecific antibody is administered subcutaneously at a dose of about 10 μg/kg to about 1000 μg/kg weekly, such as lat a dose of about 10, 20, 30, 40, 50, 60, 70, 80, 90, 100, 135, 150, 200, 250, 300, 350, 400, 405, 450, 500, 550, 600, 650, 700, 750, 800, 900, 950, 1000 μg/kg weekly. In certain other embodiments, the GPRC5D×CD3 bispecific antibody is administered subcutaneously at a dose of about 100 μg/kg to about 2400 μg/kg biweekly, such as lat a dose of about 100, 135, 150, 200, 250, 300, 350, 400, 405, 450, 500, 550, 600, 650, 700, 750, 800, 900, 950, 1000, 1100, 1200, 1300, 1400, 1500, 1600, 1700, 1800, 1900, 2000, 2100, 2200, 2300 or 2400 μg/kg, or any dose in between biweekly.

In a yet further embodiment of a method of the application, the GPRC5D×CD3 bispecific antibody is administered for a time sufficient to achieve complete response, stringent complete response, very good partial response, partial response, minimal response or stable disease status, and can be continued until disease progression or lack of patient benefit.

In a yet further embodiment of a method of the application, the GPRC5D×CD3 bispecific antibody is administered to achieve negative minimal residual disease (MRD) status, preferably negative MRD status defined as fewer than one tumor cell in 10⁻⁶ bone marrow cells, as determined by next generation sequencing (NGS), or an overall response rate of at least 20%, such as at least 20%, 25%, 30%, 35%, 40%, 45%, 50%, 55%, 60%, 65%, 70%, 75%, 80%, 85%, 90%, 95%, 100%, or any value in between.

In a yet further embodiment of a method of the application, the GPRC5D×CD3 bispecific antibody is administered to a subject in need thereof to result in an exposure of GPRC5D×CD3 bispecific antibody at a steady state mean Cmax of 10 to 25,000 ng/ml, such as 100 to 20,000 ng/ml or 1000-10,000 ng/ml, and a steady state mean AUC_{0-14d} of 1000 to 1,500,000 ng h/ml, such as 5000 to 1,000,000 ng h/ml or 10,000 to 1,000,000 ng h/mL.

In a yet further embodiment of a method of the application, the prior anti-cancer therapy is selected from the group consisting of thalidomide, lenalidomide, pomalidomide, bortezomib, ixazomib, carfilzomib, panobinostat, pamidronate, zoledronic acid, daratumumab, elotuzumab, melphalan, selinexor, belantamab mafodotin-blmf, Venetoclax, CC-92480 (CELMoD (cereblon E3 ligase modulator) agent), CAR-T therapies, other BCMA-directed therapies, other CD38-directed therapies, and combinations of two or more thereof. In some embodiment, the subject is relapsed or refractory to treatment with more than one prior anti-cancer therapies. For example, the subject can be relapsed or refractory to 2-20 prior anti-cancer therapies, such as at least two, three, four, five, six, seven, eight, nine, ten or more prior anti-cancer therapies.

In a yet further embodiment of a method of the application, the subject is a human. In certain embodiments, the subject has relapsed or refractory multiple myeloma or is intolerant to standard therapies. The subject can be previously treated with a B-cell maturation antigen (BCMA)-directed therapy. In a yet further embodiment of a method of the application, the method further comprises administering to the subject one or more additional anti-cancer therapies.

In a yet further embodiment of a method of the application, the one or more additional anti-cancer therapies are selected from the group consisting of autologous stem cell transplants (ASCT), radiation, surgery, chemotherapeutic agents, CAR-T therapies, cellular therapies, immunomodulatory agents, targeted cancer therapies, and combinations of two or more thereof.

In a yet further embodiment of any of the methods above, the one or more additional anti-cancer therapies are selected from the group consisting of selinexor, belantamab mafodotin-blmf, isatuximab, venetoclax, lenalidomide, thalidomide, pomalidomide, bortezomib, carfilzomib, elotozumab, ixazomib, melphalan, dexamethasone, vincristine, cyclophosphamide, hydroxydaunorubicin, prednisone, rituximab, imatinib, dasatinib, nilotinib, bosutinib, ponatinib, bafetinib, saracatinib, tozasertib, danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, and combinations of two or more thereof.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1A shows a schematic example of potential escalation steps.

FIG. 1B shows the study design of a phase 1 study with 184 patients in total, which includes an escalation study with weekly (QW) subcutaneous administration (SC) of 5-800 μg/kg of talquetamab, or with intravenous administration (IV) of 0.3-180 μg/kg of talquetamab, with or without step-up dosing, wherein the step-up dosing contained 1-3 step-up doses administered to the patients within 1 week before a full dose, e.g., the SC of 405 μg/kg talquetamab was administered with step-up doses of 10 and 60 μg/kg within 1 week before the administration of the full dose.

FIG. 2A is a graph showing mean PK profile following first treatment with IV dose of 60 μg/kg (n=5) and SC dose of 405 μg/kg (n=8), and FIG. 2B is a graph showing mean PK profile following first SC dose of 405 μg/kg with increased number of patients tested (n=18), wherein, EC₉₀ values were from ex vivo cytotoxicity assay using bone marrow mononuclear cells from patients with multiple myeloma (n=6) (EC₉₀, concentration of talquetamab at 90% of maximum effect; max, maximum; min, minimum).

FIG. 3A is a graph showing induction of interleukin-2 receptor subunit alpha (IL2Ra) with SC dosing with the cut-off date for the analysis of 24 Oct. 2020, and FIG. 3B is a graph showing induction of programmed cell death protein 1 (PD-1)+ T cells with SC dosing with the cut-off date for the analysis of Apr. 18, 2021.

FIG. 4 is a bar chart showing the overall response rate for SC doses, (CR, complete response; ORR, overall response rate; PR, partial response; sCR, stringent complete response; VGPR, very good partial response).

FIG. 5A is a graph showing duration of response for IV doses with the cut-off date for the analysis of 24 Oct. 2020, FIG. 5B is a graph showing duration of response for SC doses ranging from 45 to 800 μg/kg with the cut-off date for the analysis of Apr. 18, 2021, FIG. 5C is a graph showing duration of response for SC at 405 μg/kg weekly, and FIG. 5D is a graph showing duration of response for SC at 800 μg/kg biweekly (MR=minimal response; SD=stable disease; PD=progressive disease).

FIGS. 6A and 6B are graphs showing GPRC5D cell surface expression profile. Abbreviations: HD, healthy donor; NDMM, newly diagnosed MM; RRMM, relapsed/refractory MM; DARA-R MM, daratumumab-refractory MM. Bone marrow mononuclear cells (BM-MNCs) were analyzed using flow cytometry (HD n=11, MM n=74, *P<0.05; ***P<0.001; ****P<0.0001; ns=not significant).

FIG. 7 is a graph showing talquetamab-mediated MM cell lysis. Incubation of HD peripheral blood MNCs+luciferase-transduced cell line (ratio 10:1) with serial dilutions of talquetamab, Bioluminescence Imaging read out after 48 hours.

FIG. 8 is a graph showing lysis of primary MM cells. Incubation of freshly isolated BM-MNCs with serial dilutions of talquetamab or control antibodies, FACS read out after 48 hours (EC₅₀=concentration of talquetamab at 50% of maximum effect).

FIGS. 9A-9D are graphs showing impacts of pre-treatment and cytogenetic abnormalities on talquetamab-mediated lysis. Incubation of freshly isolated BM-MNCs with serial dilutions of talquetamab (n=45), FACS read out after 48 hours. RRMM: prior lines=3, 88% lenalidomide-refractory, 24% bortezomib-refractory. DARA-R MM: prior lines=6, 100% lenalidomide-refractory, 60% bortezomib-refractory, 100% daratumumab-refractory. Standard risk cytogenetics n=28, high risk n=10.

FIGS. 10A-10C are graphs showing impact of tumor and immune characteristics by talquetamab. Samples were divided in groups based on the median group-value of the indicated variable.

FIGS. 11A-11D are graphs showing impact of Treg on talquetamab efficacy. (A) Tregs and CD4+CD25− effector T-cells were isolated from healthy donor-derived buffy coats using an immune-magnetic cell isolation kit, and baseline immune cell frequencies and purity of isolated fractions were determined by flow cytometry, representative density plots are depicted; (B) Violet tracer labeled T-cells were incubated with or without Tregs for 5 days in the presence of anti-CD3/CD28 beads, proliferation was read out using flow cytometry (n=3). (C) Luciferase transduced RPMI-8226 cell line was incubated for 48 hours with 4 μg/mL of talquetamab in different conditions (n=3). (D) Cytokines and granzyme B were measured in supernatants using flow cytometry and ELISA, respectively (*P<0.05; **P<0.01, ***P<0.001; ****P<0.0001).

FIGS. 12A and 12B are graphs showing the impact of bone marrow stromal cells (BMSC) on talquetamab efficacy. (A) Luciferase-transduced MM cell lines were incubated with patient derived stromal cells (ratio 1:2)+HD PBMCs (PBMC:MM ratio 10:1) and serial dilutions of talquetamab for 48 hours. (B) Stromal cells were placed directly in the well containing MM cells and PBMCs (direct) or in a transwell insert (indirect).

FIGS. 13A-13C are graphs showing that patient-specific factors can determine response to T-cell redirectors targeting different agents. Single agent activity of both talquetamab and the BCMA-targeting bispecific antibody teclistamab (only differing in tumor-binding domain) was determined in 41 MM-patient derived BM samples (*P<0.05; **P<0.01, ns=not significant).

FIG. 14 is a graph showing the maximum Cytokine Release Syndrome (CRS) Grade in patients treated with weekly (QW) subcutaneous (SC) administration of talquetamab during the study. RP2D stands for recommended Phase 2 dose, which was administered at 405 μg/kg, with step-up doses of 10 and 60 μg/kg; CRS was graded according to Lee et al. Blood. 2014.124:188.

### DETAILED DESCRIPTION OF THE INVENTION

The disclosed methods can be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set fourth fully herein.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” as used herein refers to numerical ranges, cutoffs, or specific values means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of an assay, result or embodiment, “about” means within one standard deviation per the practice in the art, or a range of up to 5%, whichever is larger.

The term “antibodies” as used herein is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions can be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins can be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

The terms “antigen binding fragment” or “antigen binding domain” as used herein refer to a portion of an immunoglobulin molecule that binds an antigen. Antigen binding fragments can be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains can be linked together via a synthetic linker to form various types of single chain antibody designs where the VH/VL domains can pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001; WO1988/01649; WO1994/13804; and WO1992/01047.

The term “bispecific” as used herein refers to an antibody that specifically binds two distinct antigens or two distinct epitopes within the same antigen. The bispecific antibody can have cross-reactivity to other related antigens, for example to the same antigen from other species (homologs), such as human or monkey, for example Macaca cynomolgus (cynomolgus, cyno) or Pan troglodytes, or can bind an epitope that is shared between two or more distinct antigens.

Specifically binds” or “binds specifically” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a Kd of less than about 1=10⁻⁶ M, as measured by a surface plasmon resonance assay or a cell-binding assay. Phrases such as “[antigen]-specific” antibody (e.g., GPRC5D-specific antibody) are meant to convey that the recited antibody specifically binds the recited antigen.

The term “CH3 region” or “CH3 domain” as used herein refers to the CH3 region of an immunoglobulin. The CH3 region of human IgG1 antibody corresponds to amino acid residues 341-446. However, the CH3 region can also be any of the other antibody isotypes as described herein.

As used herein, a “GPRC5D×CD3 antibody” is a multispecitic antibody, optionally a bispecific antibody, which comprises two different antigen-binding regions, one of which binds specifically to the antigen GPRC5D and one of which binds specifically to CD3. A multispecitic antibody can be a bispecific antibody, diabody, or similar molecule (see for instance PNAS USA 90(14), 6444-8 (1993) for a description of diabodies). The bispecific antibodies, diabodies, and the like, provided herein can bind any suitable target in addition to a portion of GPRC5D. The term “bispecific antibody” is to be understood as an antibody having two different antigen-binding regions defined by different antibody sequences. This can be understood as different target binding but includes as well binding to different epitopes in one target.

As used herein, the terms “G-protein coupled receptor family C group 5 member D” and “GPRC5D” specifically include the human GPRCSD protein, for example as described in SEQ ID NO: 1 or GenBank Accession No. BC069341, NCBI Reference Sequence: NP 061124.1 and UniProtKB/Swiss-Prot Accession No. Q9NZD1 (see also Brauner-Osborne, H. et al. 2001, Biochim. Biophys. Acta 1518, 237-248).

SEQ ID NO: 1
MYKDCIESTGDYFLLCDAEGPWGIILESLAILGIVVTILLLLAFLFLM
RKIQDCSQWNVLPTQLLFLLSVLGLFGLAFAFIIELNQQTAPVRYFLF
GVLFALCFSCLLAHASNLVKLVRGCVSFSWTTILCIAIGCSLLQIIIA
TEYVTLEVITRGMMFVNMTPCQLNVDFVVLLVYVLFLMALTFFVSKAT
FCGPCENWKQHGRLIFITVLFSIIIWVVWISMLLRGNPQFQRQPQWDD
PVVCIALVTNAWVFLLLYIVPELCILYRSCRQECPLQGNACPVTAYQH
SFQVENQELSRARDSDGAEEDVALTSYGTPIQPQTVDPTQECFIPQAK
LSPQQDAGGV

The term “CD3” refers to the human CD3 protein multi-subunit complex. The CD3 protein multi-subunit complex is composed to 6 distinctive polypeptide chains. These include a CD37 chain (SwissProt P09693), a CD36 chain (SwissProt P04234), two CD3r chains (SwissProt P07766), and one CD3 ζ chain homodimer (SwissProt 20963), and which is associated with the T cell receptor α and β chain. The term “CD3” includes any CD3 variant, isoform and species homolog which is naturally expressed by cells (including T cells) or can be expressed on cells transfected with genes or cDNA encoding those polypeptides, unless noted. For example, human CD3 epsilon can comprise the amino acid sequence of SEQ ID NO: 2. SEQ ID NO: 3 shows the extracellular domain of a human CD3 epsilon.

SEQ ID NO: 2
MQSGTHWRVLGLCLLSVGVWGQDGNEEMGGITQTPYKVSISGTTVILT
CPQYPGSEILWQHNDKNIGGDEDDKNIGSDEDHLSLKEFSELEQSGYY
VCYPRGSKPEDANFYLYLRARVCENCMEMDVMSVATIVIVDICITGGL
LLLVYYWSKNRKAKAKPVTRGAGAGGRQRGQNKERPPPVPNPDYEPIR
KGQRDLYSGLNQRRI
SEQ ID NO: 3
DGNEEMGGITQTPYKVSISGTTVILTCPQYPGSEILWQHNDKNIGGDE
DDKNIGSDEDHLSLKEFSELEQSGYYVCYPRGSKPEDANFYLYLRARV
CENCMEMD

The term “cancer” as used herein refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” can include a tumor.

The term “combination” as used herein means that two or more therapeutics are administered to a subject together in a mixture, concurrently as single agents or sequentially as single agents in any order.

The term “complementarity determining regions” (CDR) as used herein refers to antibody regions that bind an antigen. CDRs can be defined using various delineations such as Kabat (Wu et al. J Exp Med 132: 211-50, 1970) (Kabat et al., Sequences of Proteins of Immunological Interest, 5th Ed. Public Health Service, National Institutes of Health, Bethesda, Md., 1991), Chothia (Chothia et al. J Mol Biol 196: 901-17, 1987), IMGT (Lefranc et al. Dev Comp Immunol 27: 55-77, 2003) and AbM (Martin and Thornton J Bmol Biol 263: 800-15, 1996). The correspondence between the various delineations and variable region numbering are described in, e.g., Lefranc et al. Dev Comp Immunol 27: 55-77, 2003; Honegger and Pluckthun, J Mol Biol 309:657-70, 2001; International ImMunoGeneTics (IMGT) database; and Web resources: http://www.imgt.org. Available programs such as abYsis by UCL Business PLC can be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

The term “comprising” as used herein is intended to include examples encompassed by the terms “consisting essentially of” and “consisting of”; similarly, the term “consisting essentially of” as used herein is intended to include examples encompassed by the term “consisting of” Unless the context clearly requires otherwise, throughout the description and the claims, the terms “comprise”, “comprising”, and the like are to be construed in an inclusive sense as opposed to an exclusive or exhaustive sense; that is to say, in the sense of “including, but not limited to”.

The term “enhance” or “enhanced” as used herein refers to enhancement in one or more functions of a test molecule when compared to a control molecule or a combination of test molecules when compared to one or more control molecules.

Exemplary functions that can be measured are tumor cell killing, T cell activation, relative or absolute T cell number, Fc-mediated effector function (e.g. ADCC, CDC and/or ADCP) or binding to an Fcγ receptor (FcγR) or FcRn. “Enhanced” can be an enhancement of about 10%, 20%, 30%, 40%, 50%, 60%, 70%, 80%, 90%, 100% or more, or a statistically significant enhancement.

The term “Fc gamma receptor” (FcγR) as used herein refers to well-known FcγRI, FcγRIIa, FcγRIIb or FcγRIII. Activating FcγR includes FcγRI, FcγRIIa and FcγRIII.

The term “human antibody” as used herein refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

The term “humanized antibody” as used herein refers to an antibody in which at least one CDR is derived from non-human species and at least one framework is derived from human immunoglobulin sequences. Humanized antibody can include substitutions in the frameworks so that the frameworks cannot be exact copies of expressed human immunoglobulin or human immunoglobulin germline gene sequences.

The term “isolated” as used herein refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. The term “isolated antibody” as used herein refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

The term “monoclonal antibody” as used herein refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies can have heterogeneous glycosylation within the antibody population. Monoclonal antibody can be monospecific or multispecific such as bispecific, monovalent, bivalent or multivalent.

The term “mutation” as used herein refers to an engineered or naturally occurring alteration in a polypeptide or polynucleotide sequence when compared to a reference sequence. The alteration can be a substitution, insertion or deletion of one or more amino acids or polynucleotides.

The term “multispecific” as used herein refers to an antibody that specifically binds at least two distinct antigens or at least two distinct epitopes within the same antigen. Multispecific antibody can bind for example two, three, four or five distinct antigens or distinct epitopes within the same antigen.

Current IMWG guidelines define “negative minimal residual disease status” or “negative MRD status” or “MRD negative” as fewer than one tumor cell in 100000 bone marrow cells (10⁻⁵) in a patient who fulfills the criteria for complete response (CR). Negative minimal residual disease status was determined using next generation sequencing (NGS).

The term “pharmaceutical composition” as used herein refers to composition that comprises an active ingredient and a pharmaceutically acceptable carrier. The term “pharmaceutically acceptable carrier” or “excipient” as used herein refers to an ingredient in a pharmaceutical composition, other than the active ingredient, which is nontoxic to a subject.

The term “recombinant” as used herein refers to DNA, antibodies and other proteins that are prepared, expressed, created or isolated by recombinant means when segments from different sources are joined to produce recombinant DNA, antibodies or proteins.

The term “reduce” or “reduced” as used herein refers to a reduction in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g. ADCC, CDC and/or ADCP) or binding to an Fcγ receptor (FcγR) or FcRn. “Reduced” can be a reduction of about 10%, 20%, 30%, 40%, 50%, 60%, 70%, 80%, 90%, 100% or more, or a statistically significant enhancement.

The term “refractory” as used herein refers to a cancer that is unresponsive to an anti-cancer therapy.

The term “relapsed” as used herein refers to a cancer that responded to a treatment but then returns after the treatment.

The term “subject” as used herein includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

The term “T cell redirecting therapeutic” as used herein refers to a molecule containing two or more binding regions, wherein one of the binding regions specifically binds a cell surface antigen on a target cell or tissue and wherein a second binding region of the molecule specifically binds a T cell antigen. Examples of cell surface antigen include a tumor associated antigen, such as GPRC5D. Examples of T cell antigens include, e.g., CD3. This dual/multi-target binding ability of a T cell redirecting therapeutic recruits T cells to a target cell or tissue, such as that having a tumor associated antigen, leading to the eradication of the target cell or tissue.

The term “therapeutically effective amount” as used herein refers to an amount effective, at doses and for periods of time necessary, to achieve a desired therapeutic result. A therapeutically effective amount can vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

The term “treat” or “treatment” as used herein refers to both therapeutic treatment and prophylactic or preventative measures, wherein the object is to prevent or slow down (lessen) an undesired physiological change or disorder. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

The term “tumor cell” or a “cancer cell” as used herein refers to a cancerous, pre-cancerous or transformed cell, either in vivo, ex vivo, or in tissue culture, that has spontaneous or induced phenotypic changes. These changes do not necessarily involve the uptake of new genetic material. Although transformation can arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, Md. (1991), unless otherwise explicitly stated. Antibody constant chain numbering can be found for example at ImMunoGeneTics website, at IMGT Web resources at IMGT Scientific charts.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. The application contemplates use of any of the applicable components and/or steps in any combination that can be used the application, whether or not a particular combination is expressly described.

### GPRC5D×CD3 Bispecific Antibodies and Uses Thereof

Overexpression of GPRC5D in the bone marrow is associated with poor prognosis in patients with multiple myeloma (see e.g., Atamaniuk et al., Eur. J. Clin. Invest. 42:953-960(2012)). This exclusive expression of GPRC5D on the plasma-cell lineage designates it as an ideal target for antimyeloma antibodies. Anti-GPRC5D antibodies and bispecific antibodies against GPRC5D and CD3 are described, e.g., in U.S. Pat. No. 10,562,968, the content of which is incorporated herein by reference in its entirety.

The invention is based, at least in part, on the finding that a GPRC5D×CD3 bispecific antibody, such as talquetamab, can be used to treat a hematological malignancy, such as multiple myeloma in subjects in need thereof, preferably subjects that are relapsed or refractory to treatment with a prior anti-cancer therapy. Accordingly, in one general aspect, the invention relates to a method of treating a hematological malignancy, such as multiple myeloma, in a subject in need thereof, comprising administering to the subject a therapeutically effective amount of a GPRC5D×CD3 bispecific antibody to treat the hematological malignancy, wherein the subject is relapsed or refractory to treatment with a prior anti-cancer therapy.

### Antibodies

Any suitable GPRC5D×CD3 bispecific antibody known to those skilled in the art in view of the present disclosure can be used in the invention.

Various bispecific antibody formats include formats described herein and recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule, or bispecific antibodies generated by arm exchange. Exemplary bispecific formats include dual targeting molecules include Dual Targeting (DT)-Ig (GSK/Domantis), Two-in-one Antibody (Genentech) and mAb2 (F-Star), Dual Variable Domain (DVD)-Ig (Abbott), DuoBody (Genmab), Ts2Ab (Medlmmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS) and Dual Affinity Retargeting Technology (Fc-DART) (MacroGenics), F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech), Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5):588-594.

In some embodiment, a GPRC5D×CD3 bispecific antibody useful for the invention comprises a GPRC5D binding domain comprising a heavy chain complementarity determining region 1 (HCDR1) of SEQ ID NO: 4, a HCDR2 of SEQ ID NO: 5, a HCDR3 of SEQ ID NO: 6, a light chain complementarity determining region 1 (LCDR1) of SEQ ID NO: 7, a LCDR2 of SEQ ID NO: 8 and a LCDR3 of SEQ ID NO: 9.

In other embodiment, a GPRC5D×CD3 bispecific antibody further comprises a CD3 binding domain comprising a HCDR1 of SEQ ID NO: 14, a HCDR2 of SEQ ID NO: 15, a HCDR3 of SEQ ID NO: 16, a LCDR1 of SEQ ID NO: 17, a LCDR2 of SEQ ID NO: 18, and a LCDR3 of SEQ ID NO: 19.

Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system.

In one embodiment, a GPRC5D×CD3 bispecific antibody useful for the invention comprises a GPRC5D binding domain having a heavy chain variable region (VH) of SEQ ID NO: 10 and a light chain variable region (VL) of SEQ ID NO: 11, and a CD3 binding domain having a VH of SEQ ID NO: 20 and a VL of SEQ ID NO: 21. In a yet further embodiment, a GPRC5D×CD3 bispecific antibody useful for the invention is of IgG1, IgG2, IgG3 or IgG4 isotype. In preferred embodiments, the bispecific antibody is of IgG4 isotype. An exemplary wild-type IgG4 Fc region comprises the amino acid sequence of SEQ ID NO: 24.

SEQ ID NO: 24:
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDK
RVESKYGPPCPSCPAPEFLGGPSVFLFPPKPKDTLMISRTPEVTCVVV
DVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYS
RLTVDKSRWQEGNVFSCSVMHEALHNHYTQKSLSLSLGK

A GPRC5D×CD3 bispecific antibody useful for the invention can be of any allotype. It is expected that allotype has no influence on properties of the bispecific antibodies, such as binding or Fc-mediated effector functions. Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host can be determined in part by the allotype of the antibody (Stickler et al., (2011) Genes and Immunity 12:213-21). Antibody allotype is related to amino acid sequence variations at specific locations in the constant region sequences of the antibody. Table 2 shows exemplary IgG1, IgG2 and IgG4 allotypes.

TABLE 2

| Allotype | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| | 189 | 282 | 309 | 422 | 214 | 356 | 358 | 431 |
| G2m(n) | T | M | | | | | | |
| G2m(n-) | P | V | | | | | | |
| G2m(n)/(n- | T | V | | | | | | |
| nG4m(a) | | | L | R | | | | |
| G1m(17) | | | | | K | E | M | A |
| G1m(17,1) | | | | | K | D | L | A |

In a yet further embodiment, a GPRC5D×CD3 bispecific antibody useful for the invention comprises one or more Fc substitutions that reduces binding of the bispecific antibody to a Fcγ receptor (FcγR) and/or reduces Fc effector functions such as Clq binding, complement dependent cytotoxicity (CDC), antibody-dependent cell-mediated cytotoxicity (ADCC) or phagocytosis (ADCP). The specific substitutions can be made in comparison to the wild-type IgG4 Fc region of SEQ ID NO: 24.

Fc positions that can be substituted to reduce binding of the Fc to the activating FcγR and subsequently to reduce effector function include, but are not limited to, substitutions L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4, wherein residue numbering is according to the EU index.

Fc substitutions that can be used to reduce CDC include, but are not limited to a K322A substitution. Substitutions, such as S228P substitution, can further be made in IgG4 antibodies to enhance IgG4 stability.

In a yet further embodiment, the GPRC5D×CD3 bispecific antibody can comprise one or more asymmetric substitutions in a first CH3 domain or in a second CH3 domain, or in both the first CH3 domain and the second CH3 domain.

In a yet further embodiment, the one or more asymmetric substitutions can include, but are not limited to, those selected from the group consisting of F405L/K409R, wild-type/F405L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T3661_K392M_T394W/F405A_Y407V, T366L_K392M_T394W/F405A_Y407V, L351Y_Y407A/T366A_K409F, L351Y_Y407A/T366V_K409F, Y407A/T366A_K409F and T350V_L351Y_F405A_Y407V/T350V_T366L_K392L_T394W.

In a yet further embodiment, a GPRC5D×CD3 bispecific antibody useful for the invention is of IgG4 isotype and comprises phenylalanine at position 405 and arginine at position 409 in a first heavy chain (HC1) and leucine at position 405 and lysine at position 409 in a second heavy chain (HC2).

In a yet further embodiment, a GPRC5D×CD3 bispecific antibody useful for the invention further comprises proline at position 228, alanine at position 234 and alanine at position 235 in both the HC1 and the HC2.

In a yet further embodiment, a GPRC5D×CD3 bispecific antibody useful for the invention comprises the HC1 of SEQ ID NO: 12, a first light chain (LC1) of SEQ ID NO: 13, the HC2 of SEQ ID NO: 22, and a second light chain (LC2) of SEQ ID NO: 23, wherein the LC1 binds to the HC1, the LC2 binds to the HC2, and the HC1 is linked to the HC2.

In a yet further embodiment, a GPRC5D×CD3 bispecific antibody useful for the invention is talquetamab, having the HC1 of SEQ ID NO: 12, LC1 of SEQ ID NO: 13, HC2 of SEQ ID NO: 22 and LC2 of SEQ ID NO: 23.

### Cancers

Methods of the application can be used to treat a cancer, preferably, a hematological malignancy, more preferably a relapsed or refractory hematological malignancy.

Examples of hematological malignancy can be selected from multiple myeloma, smoldering multiple myeloma, monoclonal gammopathy of undetermined significance (MGUS), acute lymphoblastic leukemia (ALL), diffuse large B-cell lymphoma (DLBCL), Burkitt's lymphoma (BL), follicular lymphoma (FL), mantle-cell lymphoma (MCL), Waldenstrom's macroglobulinema, plasma cell leukemia, light chain amyloidosis (AL), precursor B-cell lymphoblastic leukemia, precursor B-cell lymphoblastic leukemia, acute myeloid leukemia (AML), myelodysplastic syndrome (MDS), chronic lymphocytic leukemia (CLL), B cell malignancy, chronic myeloid leukemia (CML), hairy cell leukemia (HCL), blastic plasmacytoid dendritic cell neoplasm, Hodgkin's lymphoma, non-Hodgkin's lymphoma, marginal zone B-cell lymphoma (MZL), mucosa-associated lymphatic tissue lymphoma (MALT), plasma cell leukemia, anaplastic large-cell lymphoma (ALCL), leukemia or lymphoma.

In one embodiment, the hematological malignancy is multiple myeloma. In a further embodiment, the subject has a newly diagnosed multiple myeloma. In a yet further embodiment, the subject is relapsed or refractory to treatment with a prior anti-cancer therapeutic, such as a therapeutic used to treat multiple myeloma or other hematological malignancies.

In a yet further embodiment, the subject is refractory or relapsed to one or more prior anti-cancer treatments or therapies. Exemplary prior anti-cancer treatments or therapies, include, without limitation, THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (panobinostat), AREDIA® (pamidronate), ZOMETA® (zoledronic acid), DARZALEX® (daratumumab), EMPLICITI® (elotuzumab), melphalan, Xpovio® (Selinexor), BLENREP (belantamab mafodotin-blmf), Venclexta® (Venetoclax), CAR-T therapies, other BCMA-directed therapies, other CD38-directed therapies, or any combinations thereof.

Various qualitative and/or quantitative methods can be used to determine relapse or refractory nature of the disease. According to NCCN Guidelines, “clinical relapse” are defined as having one of more of the following occurred: there are direct signs of cancer growth, signs of organ damage, an increase in the number of size (at least 50% larger) of plasmacytomas or bone lesions, increased calcium levels, an increase in creatinine levels in blood, or a decrease in the number of red blood cells, and “relapse from complete response” is defined as having one or more of the following occurred in a patient who had a complete response: a return of M-proteins in blood or urine, or other signs of myeloma but not meeting the criteria for a clinical relapse progressive disease. (“Progressive disease” is defined as having one or more of the following occurred: at least 25% increase in the amount of M-proteins in the blood or urine, a 25% increase in the number of plasma cells in the bone marrow, an increase in the size or number of bone lesions, or an increase in calcium levels not explained by other conditions.) In a yet further embodiment, the multiple myeloma is a high-risk multiple myeloma. Subjects with high-risk multiple myeloma are known to relapse early and have poor prognosis and outcome. Subjects can be classified as having high-risk multiple myeloma if they have one or more of the following cytogenetic abnormalities: t(4;14)(p16;q32), t(14;16)(q32;q23), del17p, 1qAmp, t(4;14)(p16;q32) and t(14;16)(q32;q23), t(4;14)(p16;q32) and del17p, t(14;16)(q32;q23) and del17p, or t(4;14)(p16;q32), t(14;16)(q32;q23) and del17p. In some embodiments, the subject having the high-risk multiple myeloma can have one or more chromosomal abnormalities comprising: t(4;14)(p16;q32), t(14;16)(q32;q23), del17p, 1qAmp, t(4;14)(p16;q32) and t(14;16)(q32;q23), t(4;14)(p16;q32) and del17p, t(14;16)(q32;q23) and del17p; or t(4;14)(p16;q32), t(14;16)(q32;q23) and del17p, or any combination thereof.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4;14)(p16;q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF. Deletion of 17p (del17p) involves loss of the p53 gene locus.

Chromosomal rearrangements can be identified using well known methods, for example fluorescent in situ hybridization, karyotyping, pulsed field gel electrophoresis, or sequencing.

### Compositions

A GPRC5D×CD3 bispecific antibody useful for the invention can be formulated as a pharmaceutical composition comprising about 1 mg/mL to about 200 mg/mL antibody.

In one embodiment, the pharmaceutical composition further comprises one or more excipients. In some embodiments, the one or more excipients include, but are not limited to, a buffering agent, a sugar, a surfactant, a chelator, metal ion scavenger, or any combination thereof.

In a further embodiment, the pharmaceutical composition comprises:

about 1 mg/mL to about 200 mg/mL of a GPRC5D×CD3 bispecific antibody, such as about 1 mg/ml, about 5 mg/ml, about 10 mg/ml, about 15 mg/ml, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 110 mg/mL, about 120 mg/mL, or any value in between, of the GPRC5D×CD3 bispecific antibody;
about 5 mM to about 20 mM buffering agent, such as about 5 mM, about 10 mM, about 15 mM, about 20 mM, or any value in between, sodium phosphate, KH₂PO₄, sodium acetate, histidine, or sodium citrate;
about 1% w/v to about 20% w/v sugar, such as about 1% w/v, about 2% w/v, about 3% w/v, about 4% w/v, about 5% w/v, about 6% w/v, about 7% w/v, about 8% w/v, about 9% w/v, about 10% w/v, about 15% w/v, about 20% w/v, or any value in between, glucose, sucrose or cellobiose;
about 0.01% w/v to about 2% w/v surfactant, such as about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.10% w/v, about 0.5% w/v, about 1% w/v, about 1.5% w/v, about 2% w/v, or any value in between, polysorbate 80 (PS-80) or PS-20; and
about 5 mM to about 40 mM ethylenediaminetetraacetic acid (EDTA), such as about 5 mM, about 10 mM, about 15 mM, about 20 mM, about 25 mM, about 30 mM, about 35 mM, about 40 mM, or any value in between, EDTA or an edetate salt, at a pH of about 5-6, such as about 5, about 5.1, about 5.2, about 5.3, about 5.4, about 5.5, about 5.6, about 5.7, about 5.8, about 5.9, about 6, or any value in between.

The pharmaceutical composition disclosed herein may further comprise about 0.1 mg/mL to about 5 mg/mL amino acid, such as about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, or any value in between, methionine or arginine.

In one embodiment, a pharmaceutical composition useful for the invention comprises 5 mg/ml to 20 mg/ml, such as 5, 10, 15, 20 mg/ml, or any value in between, of a GPRC5D×CD3 bispecific antibody, such as talquetamab, 20 mM sodium phosphate, 10% weight/volume (w/v) sucrose, 0.06% (w/v) PS80, and 25 μg/mL EDTA at pH 5.4.

In a further embodiment, the pharmaceutical composition disclosed herein comprises 5 mg/ml to 20 mg/ml, such as 5, 10, 15, 20 mg/ml, or any value in between, of a GPRC5D×CD3 bispecific antibody, such as talquetamab, 10 to 15 mM sodium acetate, 8% (w/v) sucrose, 0.04% (w/v) PS20, and 20 μg/mL EDTA at pH 5.2.

In a yet further embodiment, the pharmaceutical composition disclosed herein comprises 5 mg/ml to 20 mg/ml, such as 5, 10, 15, 20 mg/ml, or any value in between, of a GPRC5D×CD3 bispecific antibody, such as talquetamab, 15 mM KH₂PO₄, 10% (w/v) cellobiose, 0.05% (w/v) PS20, and 25 μg/mL EDTA at pH 5.1.

In a yet further embodiment, the pharmaceutical composition disclosed herein comprises 2 mg/ml to 40 mg/ml, such as 5, 10, 15, 20, 30, 40 mg/ml, or any value in between, of a GPRC5D×CD3 bispecific antibody, such as talquetamab, 15 mM Histidine, 8% (w/v) sucrose, 0.04% (w/v) PS20, and 20 μg/mL EDTA at pH 5.2.

### Administration

In accordance with the present invention, the GPRC5D×CD3 bispecific antibody can be administered to the subject by intravenous infusion or subcutaneous injection. The dose of a GPRC5D×CD3 bispecific antibody given to a subject having a hematological malignancy, such as multiple myeloma, is sufficient to alleviate or at least partially arrest the disease being treated. Examples of the dosages useful for the invention include from about 0.2 μg/kg to about 1200 μg/kg, e.g. about 0.5 μg/kg to 100 μg/kg, about 1 μg/kg to about 800 μg/kg, about 1 μg/kg to about 500 μg/kg of the antibody. Suitable doses include, e.g., about 0.2 μg/kg, about 0.6 μg/kg, about 1.2 μg/kg, about 2.4 μg/kg, about 4.8 μg/kg, about 9.6 μg/kg, about 19.2 μg/kg, about 20 μg/kg, about 38.4 μg/kg, about 40 μg/kg, about 57.6 μg/kg, about 60 μg/kg, about 80 μg/kg, about 120 μg/kg, about 180 μg/kg, about 240 μg/kg, about 270 μg/kg, about 300 μg/kg, about 460, about 720 μg/kg, about 800 μg/kg, about 1000 μg/kg, about 1200 μg/kg, about 1600 μg/kg, about 2000 μg/kg, about 2400 μg/kg, or any dose in between.

In one embodiment, a GPRC5D×CD3 bispecific antibody is administered to a subject intravenously at a dose of about 0.2 μg/kg to about 200 μg/kg, or about 0.5 μg/kg to about 180 μg/kg, or about 1 μg/kg to about 150 μg/kg, or about 5 μg/kg to about 100 μg/kg, or about 10 μg/kg to about 70 μg/kg. Examples of the dose for intravenous administration include, e.g., about 10, 20, 30, 40, 50, 60, 70, 80, 90, 100, 110, 120, 130, 140, 150, 160, 170, 180, 190, 200 μg/kg, or any value in between. The dose can be intravenously administered monthly, tri-weekly, bi-weekly, weekly, twice weekly, or any frequency in between.

In a further embodiment, the GPRC5D×CD3 bispecific antibody is administered to a subject subcutaneously at a dose of about 0.5 μg/kg to about 2400 μg/kg, about 0.5 μg/kg to about 1200 μg/kg, or about 1 μg/kg to about 800 μg/kg, or about 10 μg/kg to about 500 μg/kg. Examples of the dose for subcutaneous administration include, e.g., about 10, 50, 100, 135, 150, 200, 250, 300, 350, 400, 405, 450, 500, 550, 600, 650, 700, 750, 800, 850, 900, 950, 1000, 1050, 1100, 1150 μg/kg, 1200 μg/kg, 1600 μg/kg, 2000 μg/kg, 2400 μg/kg, or any value in between. The dose can be subcutaneously administered monthly, tri-weekly, bi-weekly, weekly, twice weekly, or any frequency in between.

A fixed unit dose of a GPRC5D×CD3 bispecific antibody can also be given, for example, at 50, 100, 200, 500, or 1000 mg, or any value in between, per administration. The dose can also be based on the patient's surface area, e.g., 500, 400, 300, 250, 200, or 100 mg/m², or any value in between. Multiple doses can be administered to treat a hematological malignancy, such as a multiple myeloma, such as 2, 3, 4, 5, 6, 7, 8, 9, 10, 11, 12, 13, 14, 15, 16, 17, 18, 19, 20, or more doses can be given.

The administration of a GPRC5D×CD3 bispecific antibody can be repeated after one day, two days, three days, four days, five days, six days, one week, two weeks, three weeks, one month, five weeks, six weeks, seven weeks, two months, three months, four months, five months, six months, or longer. Repeated courses of treatment are also possible, as is chronic administration. The repeated administration can be at the same dose or at a different dose. For example, a GPRC5D×CD3 bispecific antibody can be administered at a first dose for a first time period, followed by administration at a second dose for a second time period. In one embodiment, the GPRC5D×CD3 bispecific antibody is administered every two weeks (i.e., bi-weekly) for a certain number of weeks, followed by administration at a second dose every week (i.e., weekly) for an additional certain number of weeks, followed by administration at a third dose every week for an additional certain number of weeks.

A GPRC5D×CD3 bispecific antibody can be administered, such as, once a week for a period needed. For example, the GPRC5D×CD3 bispecific antibody can be provided every 2 to 4 days (e.g., for the step up dosses) and then weekly, biweekly, triweekly or monthly (e.g., for the full dose) in an amount of about 0.2 μg/kg to about 2400 μg/kg, 0.2 μg/kg to about 1000 μg/kg, e.g. about 0.3 μg/kg to about 1000 μg/kg, about 0.6 μg/kg to about 600 μg/kg, about 1.2 μg/kg to about 500 μg/kg, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19, or 20 after initiation of treatment, or any combination thereof, using single or divided doses of every 24, 12, 8, 6, 4, or 2 hours, or any combination thereof.

In one embodiment, a GPRC5D×CD3 bispecific antibody is administered intravenously twice a week, once a week, once every two weeks, once every three weeks, monthly or any frequency in between in an amount of about 0.3 μg/kg, about 0.5 μg/kg, about 1.0 μg/kg, about 1.5 μg/kg, about 2.25 μg/kg, about 2.5 μg/kg, about 2.75 μg/kg, about 3 μg/kg, about 3.25 μg/kg, about 3.38 μg/kg, about 3.5 μg/kg, about 3.75 μg/kg, about 4 μg/kg, about 4.25 μg/kg, about 4.5 μg/kg, about 4.75 μg/kg, about 5p g/kg, about 7.5 μg/kg, about 10 μg/kg, about 11.25 μg/kg, about 20 μg/kg, about 30 μg/kg, about 40 μg/kg, about 50 μg/kg, about 60 μg/kg, about 80 μg/kg, about 120 μg/kg, about 180 μg/kg, about 200 μg/kg, or any dose in between.

In a further embodiment, a GPRC5D×CD3 bispecific antibody is administered subcutaneously twice a week, once a week, once every two weeks, once every three weeks, monthly or any frequency in between in an amount of about 0.3 μg/kg, about 0.6 μg/kg, about 1.2 μg/kg, about 1.5 μg/kg, about 2.5 μg/kg, about 5 μg/kg, about 10 μg/kg, about 15 μg/kg, about 20 μg/kg, about 25 μg/kg, about 30 μg/kg, about 40 μg/kg, about 45 μg/kg, about 50 μg/kg, about 55 μg/kg, about 60 μg/kg, about 80 μg/kg, about 120 μg/kg, about 135 μg/kg, about 180 μg/kg, about 240 μg/kg, about 270 μg/kg, about 300 μg/kg, about 350 μg/kg, about 400 μg/kg, about 405 μg/kg, about 720 μg/kg, about 800 μg/kg, about 1000 μg/kg, about 1200 μg/kg, about 1600 μg/kg, about 2000 μg/kg, about 2400 μg/kg, or any dose in between.

In some embodiments, a GPRC5D×CD3 bispecific antibody is administered in one or more priming administrations that gradually increase the dose levels. The priming dose strategies can be utilized effectively for bispecific T cell engager antibodies such as a GPRC5D×CD3 bispecific antibody, due to the potential for these antibodies to cause more pronounced acute toxicities with the initial dose. One or more priming doses, can be used to ensure safety, obtain the desired T cell adaptation effect, decrease cytokine levels, and decrease the incidence of symptomatic cytokine release syndrome (CRS) in a majority of treated subjects. Priming dose(s) is administered prior to Day 1 of the bi-weekly, weekly, triweekly or monthly dose schedules with higher dose.

Accordingly, in one embodiment, a GPRC5D×CD3 bispecific antibody is administered intravenously at a step-up (or “priming”) dose, followed by weekly, biweekly, triweekly, or monthly administration at a higher dose intravenously or subcutaneously. For example, the GPRC5D×CD3 bispecific antibody can be administered intravenously at a priming dose of about 0.3 μg/kg, about 0.5 μg/kg, about 0.6 μg/kg, about 1.0 μg/kg, about 1.5 μg/kg, about 2.25 μg/kg, about 2.4 μg/kg, about 3.0 μg/kg, about 3.38 μg/kg, about 3.5 μg/kg, about 3.75 μg/kg, about 4 μg/kg, about 4.25 μg/kg, about 4.5 μg/kg, about 4.75 μg/kg, about 5 μg/kg, or any dose in between. After the priming administration, the GPRC5D×CD3 bispecific antibody can be administered weekly, biweekly, triweekly or monthly intravenously at a higher dose, such as about 1.0 μg/kg, about 1.5 μg/kg, about 2.25 μg/kg, about 2.5 μg/kg, about 2.75 μg/kg, about 3 μg/kg, about 3.25 μg/kg, about 3.38 μg/kg, about 3.5 μg/kg, about 3.75 μg/kg, about 4 μg/kg, about 4.25 μg/kg, about 4.5 μg/kg, about 4.75 μg/kg, about 5 μg/kg, about 7.5 μg/kg, about 10 μg/kg, about 11.25 μg/kg, about 20 μg/kg, about 30 μg/kg, about 40 μg/kg, about 50 μg/kg, about 60 μg/kg, about 80 μg/kg, about 120 μg/kg, about 180 μg/kg, about 200 μg/kg, or any dose in between.

In another embodiment, after the priming administration, the GPRC5D×CD3 bispecific antibody is administered weekly, biweekly, triweekly or monthly subcutaneously at a higher dose, such as about 1.2 μg/kg, about 1.5 μg/kg, about 2.5 μg/kg, about 5 μg/kg, about 10 μg/kg, about 15 μg/kg, about 20 μg/kg, about 25 μg/kg, about 30 μg/kg, about 40 μg/kg, about 45 μg/kg, about 50 μg/kg, about 55 μg/kg, about 60 μg/kg, about 80 μg/kg, about 120 μg/kg, about 135 μg/kg, about 180 μg/kg, about 240 μg/kg, about 270 μg/kg, about 300 μg/kg, about 350 μg/kg, about 400 μg/kg, about 405 μg/kg, about 720 μg/kg, about 800 μg/kg, about 1000 μg/kg, about 1200 μg/kg, about 1600 μg/kg, about 2000 μg/kg, about 2400 μg/kg, or any dose in between.

In a yet further embodiment, a GPRC5D×CD3 bispecific antibody is administered intravenously at a first step-up dose, followed by administration at a second higher step-up dose, followed by weekly, biweekly, triweekly or monthly administration at a third, higher dose. For example, the GPRC5D×CD3 bispecific antibody can be administered intravenously at a step-up dose of about 0.5 μg/kg, about 1.0 μg/kg, about 1.5 μg/kg, about 2.25 μg/kg, about 2.5 μg/kg, about 2.75 μg/kg, about 3.0 μg/kg, about 3.25 μg/kg, about 3.5 μg/kg, or any dose in between, followed by intravenous administration at a second step-up dose of about 5 μg/kg, about 7.5 μg/kg, about 10 μg/kg, about 12.5 μg/kg, about 15 μg/kg, or any dose in between, followed by weekly intravenous administration at a dose of about 15 μg/kg, about 20 μg/kg, about 30 μg/kg, about 40 μg/kg, about 50 μg/kg, about 60 μg/kg, about 80 μg/kg, about 120 μg/kg, about 180 μg/kg, or any dose in between.

In a yet further embodiment, the GPRC5D×CD3 bispecific antibody is administered intravenously at a first step-up dose, followed by administration at a second higher step-up dose, followed by administration at a third higher step-up dose, followed by weekly, biweekly, triweekly or monthly administration at a fourth, higher dose. For example, the GPRC5D×CD3 bispecific antibody can be administered intravenously at a first step-up dose of about 0.3 μg/kg, about 0.6 μg/kg, about 1.2 μg/kg, about 1.5 μg/kg, about 1.75 μg/kg, about 2.0 μg/kg, about 2.25 μg/kg, about 2.5 μg/kg, about 2.75 μg/kg, about 3 μg/kg, or any dose in between, followed by intravenous administration at a second step-up dose of about 5 μg/kg, about 7.5 μg/kg, about 10 μg/kg, about 12.5 μg/kg, about 15 μg/kg, or any dose in between, followed by intravenous administration at a step-up dose of about 40 μg/kg, about 50 μg/kg, about 60 μg/kg, about 70 μg/kg, about 80 μg/kg, or any dose in between, followed by weekly, biweekly, triweekly or monthly intravenous administration at a dose of about 150 μg/kg, about 180 μg/kg, about 200 μg/kg, or any dose in between.

In a yet further embodiment, the GPRC5D×CD3 bispecific antibody is administered subcutaneously at a step-up dose, followed by weekly, biweekly, triweekly or monthly administration at a higher dose. For example, the GPRC5D×CD3 bispecific antibody can be administered subcutaneously at a step-up dose of about 1.5 μg/kg, about 5 μg/kg, about 10 μg/kg, about 20 μg/kg, about 40 μg/kg, about 45 μg/kg, about 60 μg/kg, or any dose in between, followed by weekly subcutaneously administration at a dose of about 5 μg/kg, about 10 μg/kg, about 15 μg/kg, about 20 μg/kg, about 25 μg/kg, about 30 μg/kg, about 40 μg/kg, about 45 μg/kg, about 50 μg/kg, about 55 μg/kg, about 60 μg/kg, about 80 μg/kg, 120 μg/kg, about 135 μg/kg, about 180 μg/kg, about 240 μg/kg, about 300 μg/kg, about 270 μg/kg, about 360 μg/kg, about 400 μg/kg, about 405 μg/kg, about 420 μg/kg, about 480 μg/kg, about 540 μg/kg, about 600 μg/kg, about 760 μg/kg, about 920 μg/kg, about 1000 μg/kg, about 1200 μg/kg, about 1600 μg/kg, about 2000 μg/kg, about 2400 μg/kg, or any dose in between.

In a yet further embodiment, a GPRC5D×CD3 bispecific antibody is administered subcutaneously at a first step-up dose, followed by administration at a second higher step-up dose, followed by weekly, biweekly, triweekly or monthly administration at a third, higher dose. For example, the GPRC5D×CD3 bispecific antibody can be administered subcutaneously at a step-up dose of about 1.5 μg/kg, about 5 μg/kg, about 10 μg/kg, about 15 μg/kg, or any dose in between, followed by subcutaneously administration at a higher step-up dose of about 30 μg/kg, about 40 μg/kg, about 45 μg/kg, about 60 μg/kg or any dose in between, followed by weekly subcutaneously administration at a dose of about 100 μg/kg, about 135 μg/kg, about 240 μg/kg, about 300 μg/kg, about 400 μg/kg, about 405 μg/kg, about 800 μg/kg, about 1000 μg/kg, about 1200 μg/kg, about 1600 μg/kg, about 2000 μg/kg, about 2400 μg/kg or any dose in between.

In a yet further embodiment, a GPRC5D×CD3 bispecific antibody is administered subcutaneously at a first step-up dose, followed by administration at a second higher step-up dose, followed by administration at a third, higher step-up dose, followed by weekly, biweekly, triweekly or monthly administration at a fourth, higher dose. For example, the GPRC5D×CD3 bispecific antibody can be administered subcutaneously at a first step-up dose of about 1.5 μg/kg, about 4 μg/kg, about 6 μg/kg, about 8 μg/kg, about 10 μg/kg, about 12 μg/kg, about 14 μg/kg, about 16 μg/kg, about 18 μg/kg, about 20 μg/kg, or any dose in between, followed by subcutaneously administration at a second step-up dose of about 30 μg/kg, about 45 μg/kg, about 60 μg/kg, about 75 μg/kg, about 100 μg/kg, or any dose in between, followed by subcutaneously administration at a third step-up dose of about 150 μg/kg, about 200 μg/kg, about 250 μg/kg, about 300 μg/kg, about 350 μg/kg, about 400 μg/kg, or any dose in between, followed by weekly subcutaneous administration at a dose of about 500 μg/kg, 600 μg/kg, about 700 μg/kg, about 800 μg/kg, about 900 μg/kg, about 1000 μg/kg, about 1200 μg/kg, about 1600 μg/kg, about 2000 μg/kg, about 2400 μg/kg, or any dose in between.

In a yet further embodiment, a GPRC5D×CD3 bispecific antibody is administered for a time sufficient to achieve complete response, stringent complete response, very good partial response, partial response, minimal response or stable disease status, and can be continued until disease progression or lack of patient benefit. The disease status can be determined by any suitable method known to those skilled in the art in view of the present disclosure, including, e.g., analysis of serum and urine monoclonal protein concentrations, M-protein levels, GPRC5D levels.

In certain embodiments, a GPRC5D×CD3 bispecific antibody is administered for a time sufficient to achieve complete response that is characterized by negative minimal residual disease (MRD) status. Negative MRD status can be determined by any method suitable method known to those skilled in the art in view of the present disclosure. In some embodiments, negative MRD status is determined using next generation sequencing (NGS). In other embodiments, negative MRD status is determined using EuroFlow, a sensitive flow cytometric test. In some embodiments, negative MRD status is determined at 10⁻⁴ cells, 10⁻⁵ cells, or 10⁻⁶ cells. In some embodiment, the administration of GPRC5D×CD3 can be continued after the negative MRD status is achieved as a maintenance therapy. In another embodiment, the administration of GPRC5D×CD3 is discontinued after the negative MRD status is achieved.

A GPRC5D×CD3 bispecific antibody can also be administered prophylactically in order to reduce the risk of developing cancer, such as smoldering multiple myeloma (SMM), delay the onset of the occurrence of an event in cancer progression, and/or reduce the risk of recurrence when the cancer is in remission.

### Combinations

In certain embodiments, a method of the application further comprises administering to the subject one or more other anti-cancer therapies.

The one or more other anti-cancer therapies can include, without limitation, autologous stem cell transplants (ASCT), radiation, surgery, chemotherapeutic agents, CAR-T therapies, cellular therapies, immunomodulatory agents, targeted cancer therapies, and any combination thereof. In certain embodiments, a method of the application further comprises administering to the subject a therapy that reduces or depletes Treg, such as low-dose cyclophosphamide.

The one or more other anti-cancer therapies can also include, without limitation, selinexor, belantamab mafodotin-blmf, isatuximab, venetoclax, lenalidomide, thalidomide, pomalidomide, bortezomib, carfilzomib, elotozumab, ixazomib, melphalan, CC-92480, dexamethasone, vincristine, cyclophosphamide, hydroxydaunorubicin, prednisone, rituximab, imatinib, dasatinib, nilotinib, bosutinib, ponatinib, bafetinib, saracatinib, tozasertib, danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, and any combination thereof.

Thus, provided herein is a combination of an effective amount of a GPRC5D×CD3 bispecific antibody, and an effective amount of other anti-cancer therapies for use in treating a hematological malignancy, such as MM, preferably a MM that is relapsed or refractory to a prior anti-cancer therapy.

As used herein, the terms and phrases “in combination,” “in combination with,” “co-delivery,” and “administered together with” in the context of the administration of two or more therapies or components to a subject refers to simultaneous administration, overlapping administration or subsequent administration of two or more therapies or components. “Simultaneous administration” or “simultaneously administered” refers to administration of the two or more therapies or components within the same treatment period. When two components are administered “within the same treatment period,” they can be administered in separate compositions according to their own administration schedules, as long as the periods of administration for the two components end around the same day or within a short time period, such as within 1 day, 1 week, or 1 month. “Overlapping administration” refers to administration of the two or more therapies or components not within the same overall treatment period, but with at least one overlapping treatment period. “Subsequent administration” refers to administration of the two or more therapies or components during different treatment periods, one after the other. The use of the term “in combination with” does not restrict the order in which therapies or components are administered to a subject. For example, a first therapy or component can be administered prior to, concomitantly with or simultaneously with, or subsequent to the administration of a second therapy or component.

The term “BLRM model” refers to the Bayesian logistic regression model as described in Neuenschwander et al. Sta tMed. 2008. 27(13): 2420-39. In Part 1 (dose escalation), the probability of dose-limiting toxicities (DLTs) from a BLRM with the EWOC (Escalation with overdose control) principle guides the dose escalation. The BLRM with the EWOC principle will also be implemented in Part 2 (dose expansion). The following two-parameter BLRM is central to the calculation of the probability of DLTs when a subject's planned maximum dose during the firstcyeis d:

logit( )=α+β log(d/d*);α∈ [symbol] ,β>0

where, π is the probability that a DLT occurs during the DLT evaluation period when talquetamab is given as a single agent with planned maximum dose=d during the first cycle,

$$logit ( ) = log ( \frac{π}{1 - π} ) ,$$

and d* is a reference planned maximum dose during the first cycle.

While having described the invention in general terms, the embodiments of the invention will be further disclosed in the following examples that should not be construed as limiting the scope of the claims.

### Examples

The following examples are provided to further describe some of the embodiments disclosed herein. The examples are intended to illustrate, not to limit, the disclosed embodiments.

### Antibodies and Reagents

A fully humanized IgG4 anti-GPRC5D/anti-CD3 bispecific antibody talquetamab (described in U.S. Pat. No. 10,562,968, the content of which is incorporated herein by reference in its entirety) was made by Janssen Pharmaceuticals. It was produced by cultivation of recombinant Chinese Hamster Ovary cells followed by isolation, chromatographic purification, and formulation. Talquetamab comprises a GPRC5D binding arm GC5B596 and a CD3 binding arm CD3B219, the amino acid sequences of which are shown in Table 3 and Table 4, respectively.

TABLE 3
Sequences of GPRC5D binding arm of Talquetamab

| | | | SEQ |
|---|---|---|---|
| | | | ID |
| | Region | Sequence | NO: |
| GC5B596 | HCDR1 | GYTMN | 4 |
| | HCDR2 | LINPYNSDTNYAQKLQG | 5 |
| | HCDR3 | VALRVALDY | 6 |
| | LCDR1 | KASQNVATHVG | 7 |
| | LCDR2 | SASYRYS | 8 |
| | LCDR3 | QQYNRYPYT | 9 |
| | VH | QVQLVQSGAEVKKPGASVKVSCKASGYSF | 10 |
| | | TGYTMNWVRQAPGQGLEWMGLINPYNSD | |
| | | TNYAQKLQGRVTMTTDTSTSTAYMELRSL | |
| | | RSDDTAVYYCARVALRVALDYWGQGTLV | |
| | | TVSS | |
| | VL | DIQMTQSPSSLSASVGDRVTITCKASQNVA | 11 |
| | | THVGWYQQKPGKAPKRLIYSASYRYSGVP | |
| | | SRFSGSGSGTEFTLTISNLQPEDFATYYCQQ | |
| | | YNRYPYTFGQGTKLEIK | |
| | HC | QVQLVQSGAEVKKPGASVKVSCKASGYSF | 12 |
| | | TGYTMNWVRQAPGQGLEWMGLINPYNSD | |
| | | TNYAQKLQGRVTMTTDTSTSTAYMELRSL | |
| | | RSDDTAVYYCARVALRVALDYWGQGTLV | |
| | | TVSSASTKGPSVFPLAPCSRSTSESTAALGC | |
| | | LVKDYFPEPVTVSWNSGALTSGVHTFPAV | |
| | | LQSSGLYSLSSVVTVPSSSLGTKTYTCNVD | |
| | | HKPSNTKVDKRVESKYGPPCPPCPAPEAAG | |
| | | GPSVFLFPPKPKDTLMISRTPEVTCVVVDV | |
| | | SQEDPEVQFNWYVDGVEVHNAKTKPREE | |
| | | QFNSTYRVVSVLTVLHQDWLNGKEYKCK | |
| | | VSNKGLPSSIEKTISKAKGQPREPQVYTLPP | |
| | | SQEEMTKNQVSLTCLVKGFYPSDIAVEWE | |
| | | SNGQPENNYKTTPPVLDSDGSFFLYSRLTV | |
| | | DKSRWQEGNVFSCSVMHEALHNHYTQKS | |
| | | LSLSLGK | |
| | LC | DIQMTQSPSSLSASVGDRVTITCKASQNVA | 13 |
| | | THVGWYQQKPGKAPKRLIYSASYRYSGVP | |
| | | SRFSGSGSGTEFTLTISNLQPEDFATYYCQQ | |
| | | YNRYPYTFGQGTKLEIKKAAPSVTLFPPSSE | |
| | | ELQANKATLVCLISDFYPGAVTVAWKGDS | |
| | | SPVKAGVETTTPSKQSNNKYAASSYLSLTP | |
| | | EQWKSHRSYSCQVTHEGSTVEKTVAPTEC | |
| | | S | |

TABLE 4
Sequences of CD3 binding arm of talquetamab

| | | | SEQ |
|---|---|---|---|
| | | | ID |
| | Region | Sequence | NO: |
| CD3B219 | HCDR1 | TYAMN | 14 |
| | HCDR2 | RIRSKYNNYATYYAASVKG | 15 |
| | HCDR3 | HGNFGNSYVSWFAY | 16 |
| | LCDR1 | RSSTGAVTTSNYAN | 17 |
| | LCDR2 | GTNKRAP | 18 |
| | LCDR3 | ALWYSNLWV | 19 |
| | VH | EVQLVESGGGLVQPGGSLRLSCAASGFTENT | 20 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTL | |
| | | VTVSS | |
| | VL | QTVVTQEPSLTVSPGGTVTLTCRSSTGAVTT | 21 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQP | |
| | HC | EVQLVESGGGLVQPGGSLRLSCAASGFTENT | 22 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTL | |
| | | VTVSSASTKGPSVFPLAPCSRSTSESTAALG | |
| | | CLVKDYFPEPVTVSWNSGALTSGVHTFPAVL | |
| | | QSSGLYSLSSVVTVPSSSLGTKTYTCNVDHK | |
| | | PSNTKVDKRVESKYGPPCPPCPAPEAAGGPS | |
| | | VFLEPPKPKDTLMISRTPEVTCVVVDVSQED | |
| | | PEVQFNWYVDGVEVHNAKTKPREEQFNSTY | |
| | | RVVSVLTVLHQDWLNGKEYKCKVSNKGLPS | |
| | | SIEKTISKAKGQPREPQVYTLPPSQEEMTKN | |
| | | QVSLTCLVKGFYPSDIAVEWESNGQPENNYK | |
| | | TTPPVLDSDGSFLLYSKLTVDKSRWQEGNVF | |
| | | SCSVMHEALHNHYTQKSLSLSLGK | |
| | LC | QTVVTQEPSLTVSPGGTVTLTCRSSTGAVTT | 23 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQPKAAPSVTLFP | |
| | | PSSEELQANKATLVCLISDFYPGAVTVAWKA | |
| | | DSSPVKAGVETTTPSKQSNNKYAASSYLSLT | |
| | | PEQWKSHRSYSCQVTHEGSTVEKTVAPTECS | |

### Example 1: Mechanisms of Resistance and Determinants of Response of Talquetamab in Multiple Myeloma

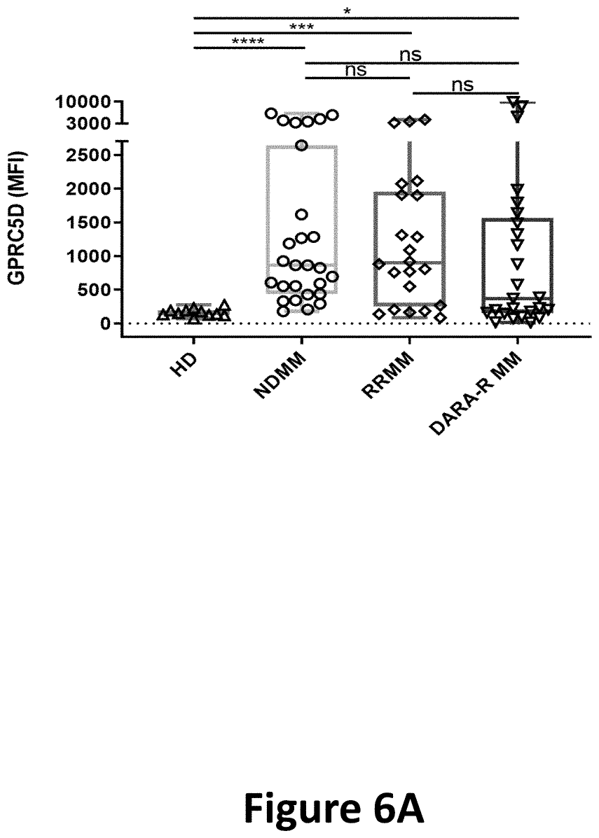

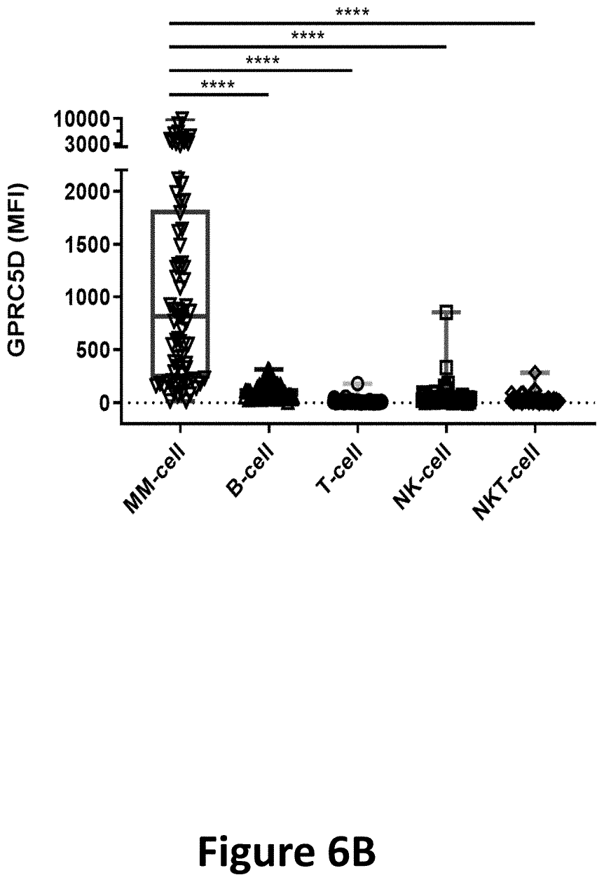

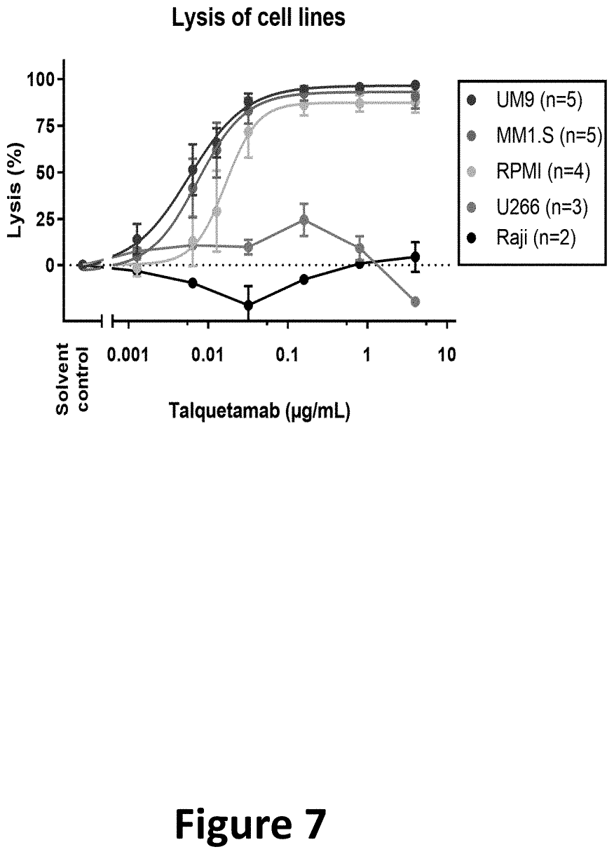

In this study, it is demonstrated that GPRC5D cell surface expression, measured by flow cytometry, is significantly higher on malignant plasma cells in different stages of the disease (newly diagnosed (ND), relapsed/refractory (RR), and daratumumab-refractory (DARA-R)) than on normal plasma cells from healthy donors (FIG. 6A). GPRC5D expression is also higher on MMN cells than on other immune cells (FIG. 6B). This selective expression renders it an attractive target for immunotherapy. In addition, talquetamab showed substantial activity in GPRC5D+ cell lines (FIG. 7).

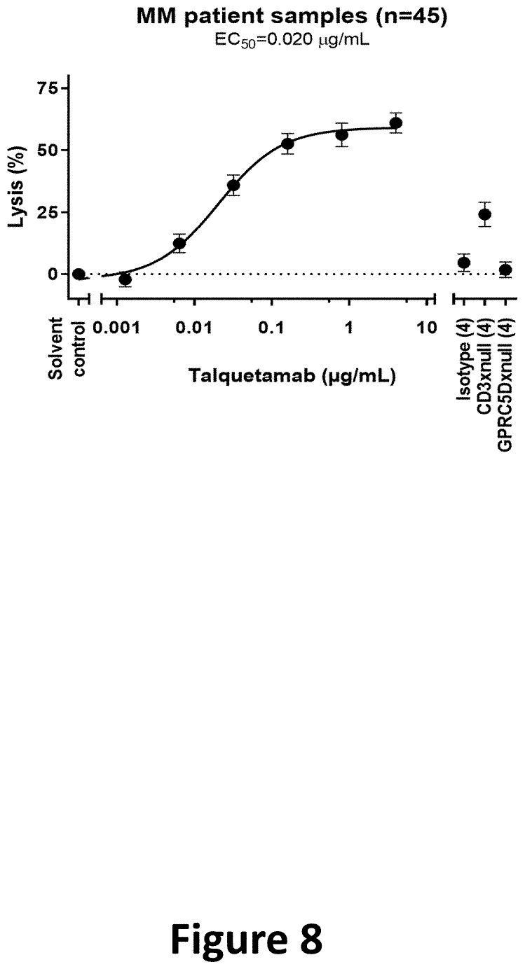

Next, bone marrow mononuclear cells (BM-MNCs) from 45 MM patients (containing MM cells, effector cells and immunosuppressive cells) were isolated and analyzed tumor- and immune cell characteristics by flow cytometry. Subsequently, cells were incubated with serial dilutions of talquetamab for 48 hours, after which lysis of CD138+ MM cells was determined by flow cytometry. Mean lysis at the highest dose was 61% (FIG. 8), but ranged from −5 to 97%. Mechanisms of resistance and determinants of response of talquetamab were then further investigated.

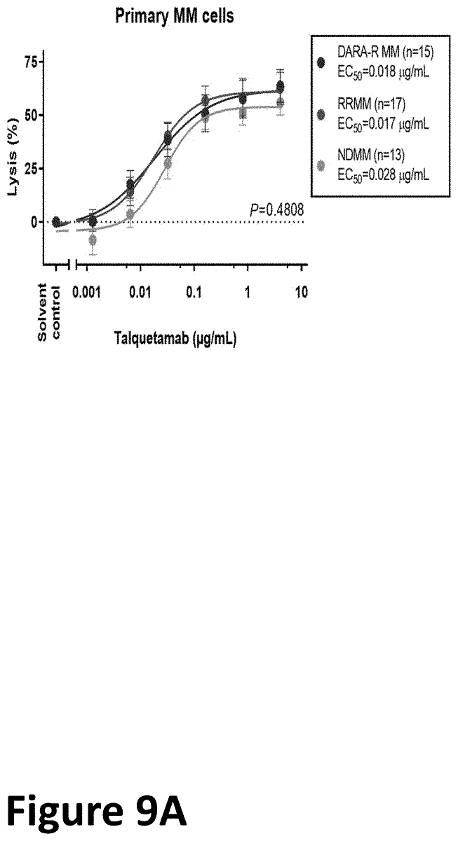

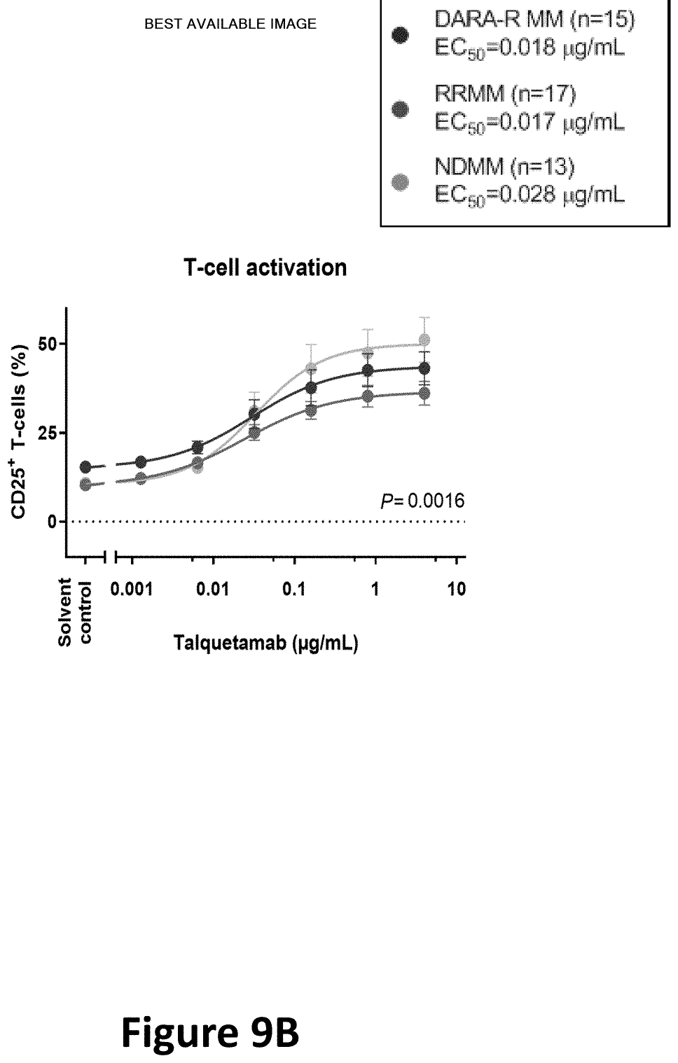

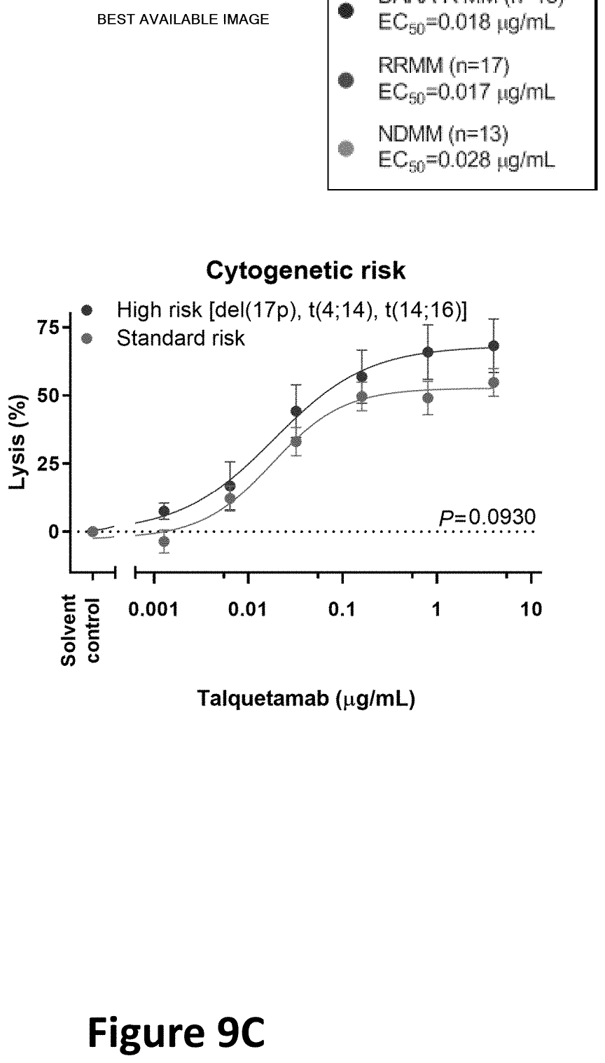

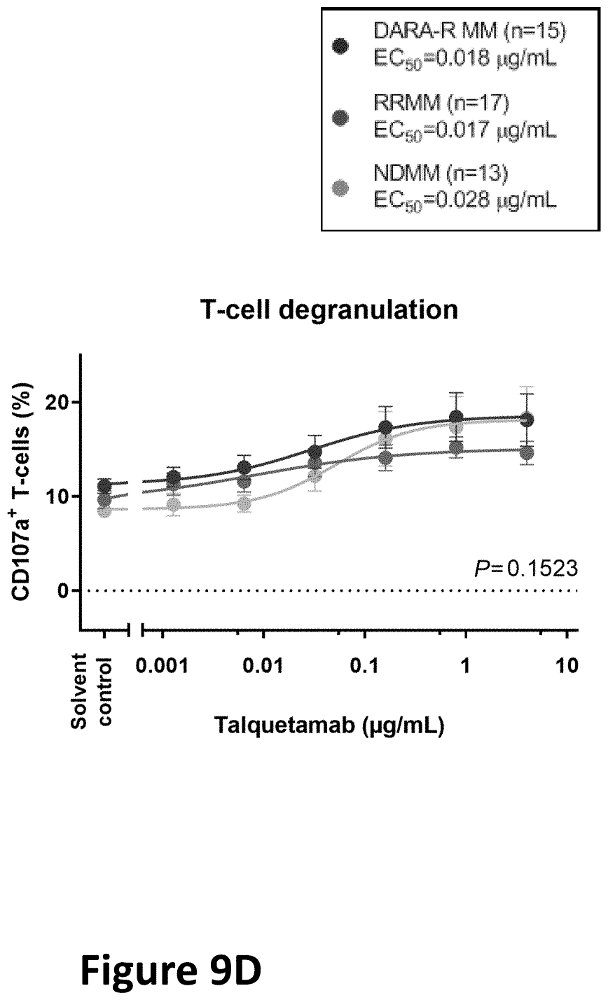

It was further found that pre-treatment and cytogenetic abnormalities have no impact on talquetamab-mediated lysis (FIGS. 9A-9D). Specifically, no difference was found in dose-response curves between ND− dara naive RR- and DARA-R MM patients. Although talquetamab-mediated T-cell activation (defined by CD25+) in samples derived from ND patients was slightly higher than in dara naive RR patients, there was no difference in T-cell degranulation (as defined by CD107+) between groups. Importantly, the presence of high-risk cytogenetic abnormalities did not impair talquetamab-mediated MM cell lysis. This indicates that heavily pretreated and/or high-risk patients may benefit from GPRC5D-targeting bispecific antibody therapy.

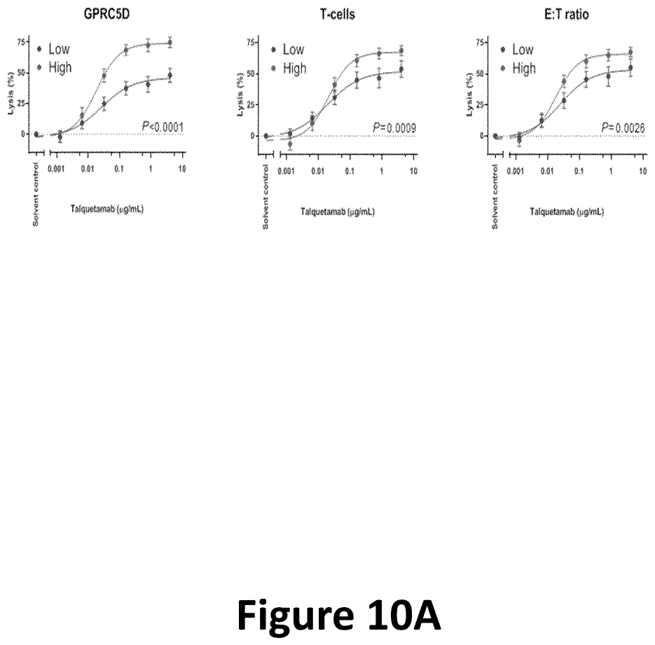

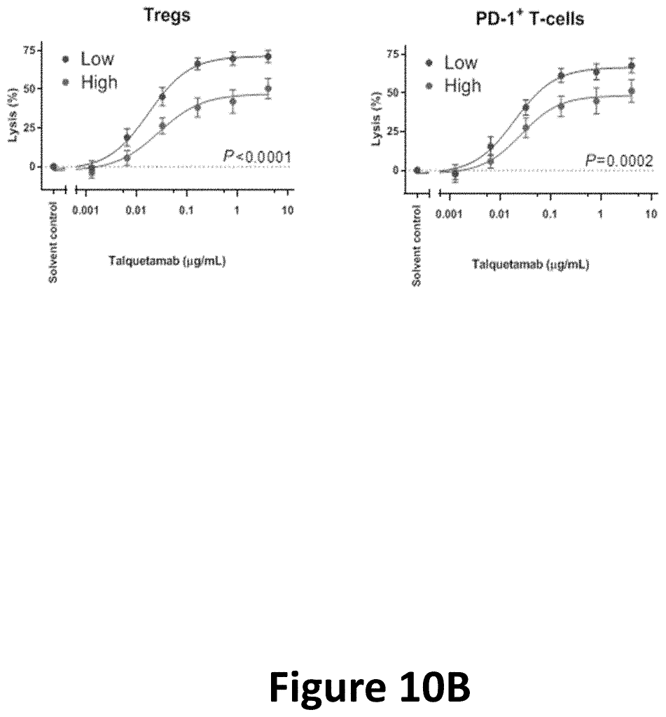

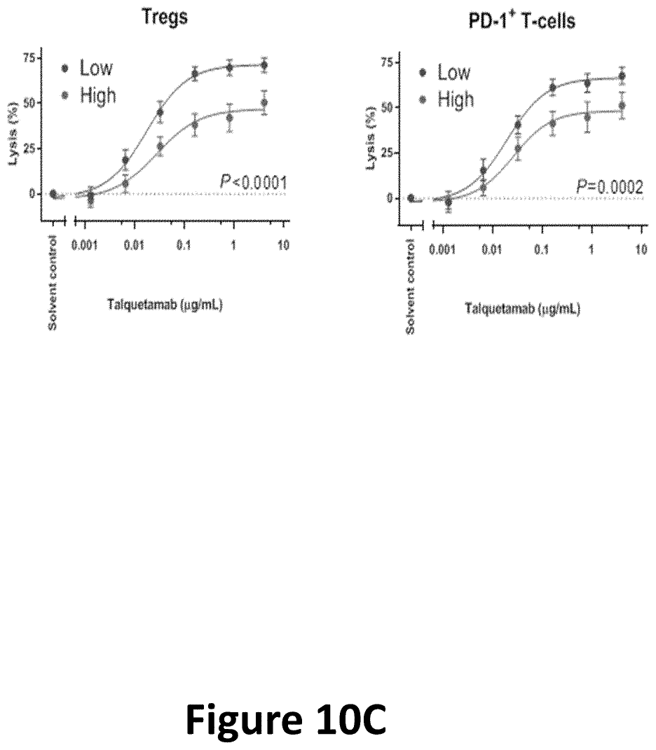

FIGS. 10A-10C shows the impact of tumor and immune characteristics. The level of target expression was an important determinant of response, as evidenced by superior MM cell lysis in samples with higher than median GPRC5D expression (in darker dots), when compared to lower GPRC5D expression (in lighter dots). Inferior MM cell lysis was observed in samples with low T-cell counts or low effector:target (E:T) ratios, and in those with a high frequency of Tregs, PD-1+ T-cells, HLA-DR+ activated T-cells, and in older patients. These determinants of response also affected talquetamab mediated T-cell activation and degranulation. The variability of GPR5D expression and Treg count most affected talquetamab-efficacy.

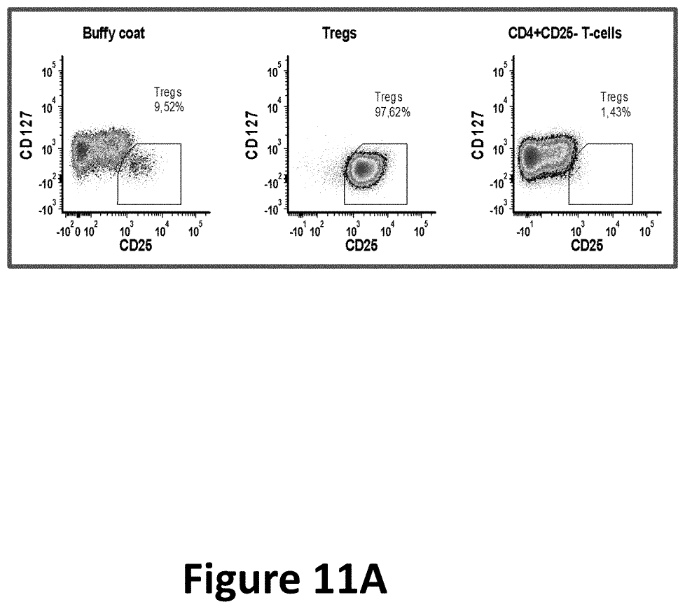

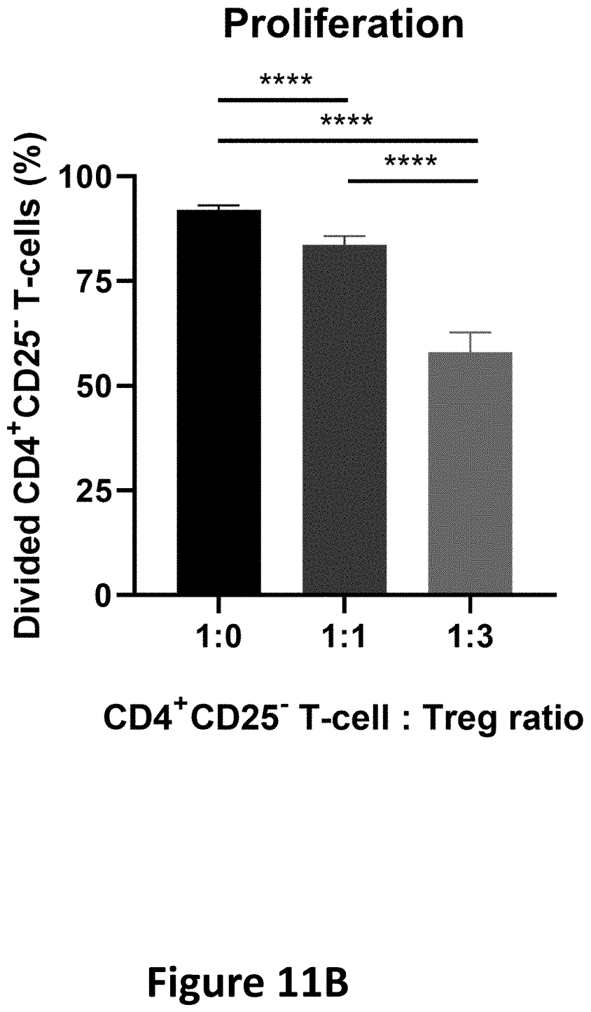

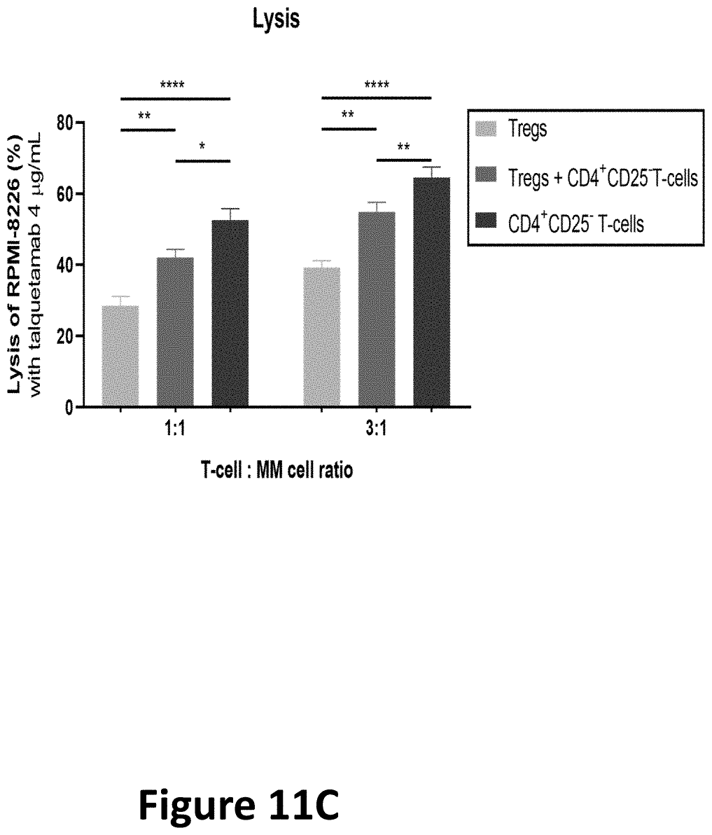

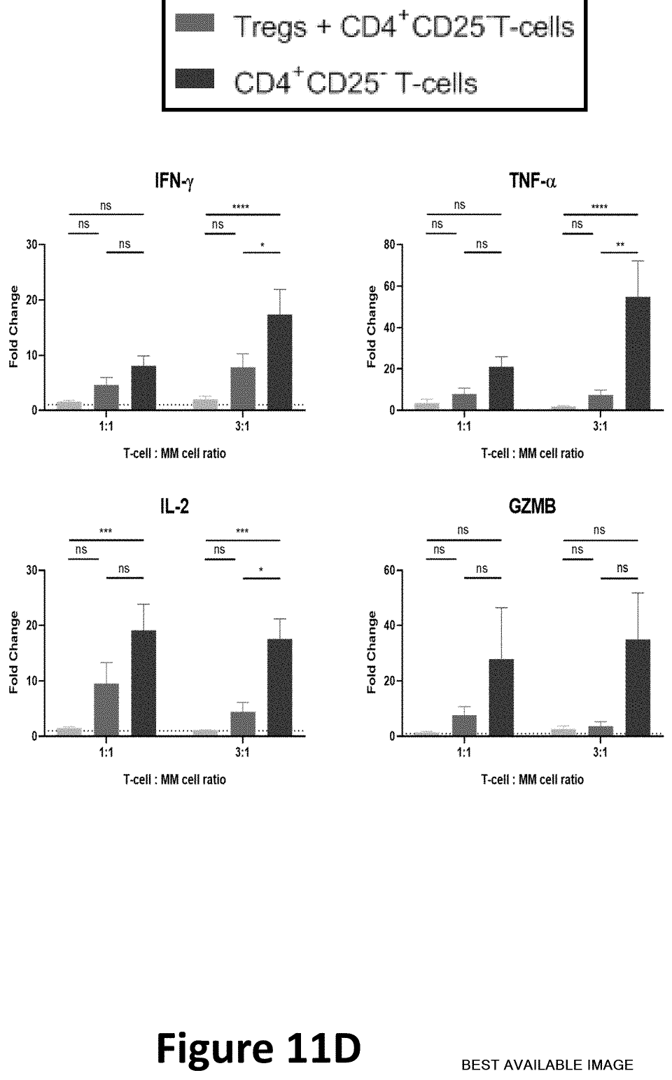

As shown in FIGS. 11A-11D, additional cell line experiments were performed and the impact of Tregs on talquetamab efficacy was further investigated. Tregs and CD4+CD25− effector T cells were purified from a buffy coat. Tregs impaired T-cell proliferation, confirming their suppressive function. Tregs were significantly less potent to kill MM cells when redirected by talquetamab, compared to CD4+CD25− T-cells (FIG. 11C). This was accompanied by reduced secretion of IFN-γ, TNF-α, IL-2 and granzyme B. Patients with high Treg counts may benefit from Treg depletion strategies, such as low-dose cyclophosphamide.

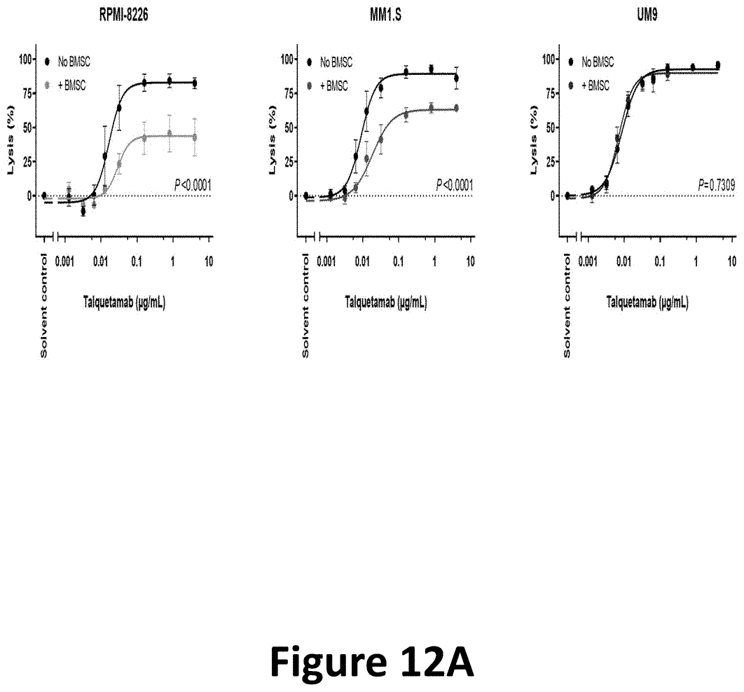

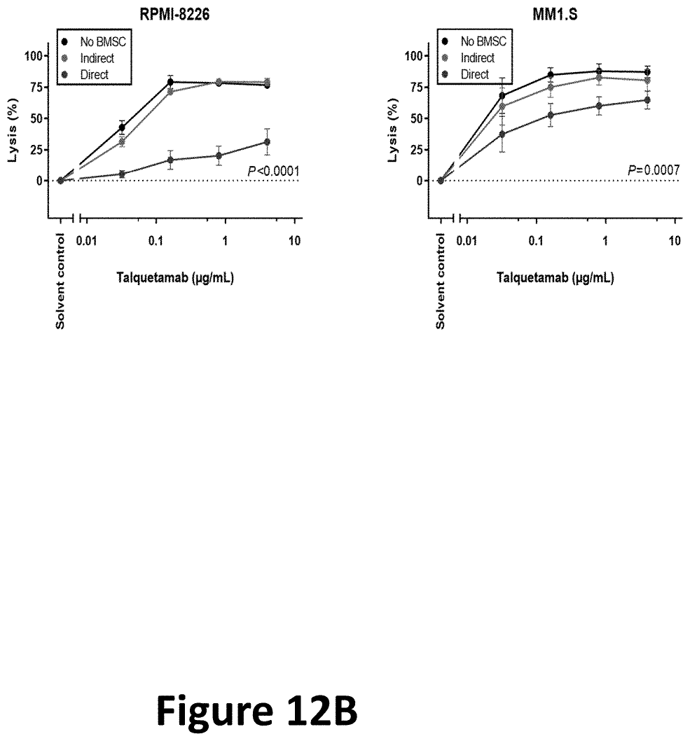

To evaluate the impact of BM stromal cells (BMSCs) on talquetamab activity, MM cell lines were co-incubated with PBMCs and patient-derived BMSCs. Direct cell-cell contact hampered MM cell lysis, while indirect contact (transwell) did not affect talquetamab activity (FIGS. 12A and 12B). This indicates that cell-cell contact is required to reduce talquetamab-mediated MM cell lysis, whereas BMSC-derived soluble factors alone do not impair the activity of talquetamab. The protection conferred by BMSCs against talquetamab-mediated lysis may be due to acquired resistance of MM cells (e.g., altered target expression following adhesive interactions) and/or T-cell suppression. Additional experiments showed that contact with BMSCs resulted in reduced expression of GPRC5D on RPMI-8226 and MM.1 S cells, but not on UM9 cells, correlating with BMSC-mediated talquetamab resistance of RPMI-8226 and MM.1 S, but not of UM9. However, talquetamab-mediated lysis was inhibited by BMSCs without a reduction in T-cell activation and degranulation, indicating the induction of cell intrinsic resistance mechanisms against the cytotoxic machinery of T cells by BMSCs. Similar results were obtained when CD4 and CD8 T cells were analyzed separately. While not wishing to be bound by theories, direct contact of MM cells with BMSCs contributed to the induction of talquetamab resistance, at least in part through reducing GPRC5D expression on MM cells. Combination strategies with established anti-MM agents could improve the efficacy of GPRC5D-targeting bispecific.

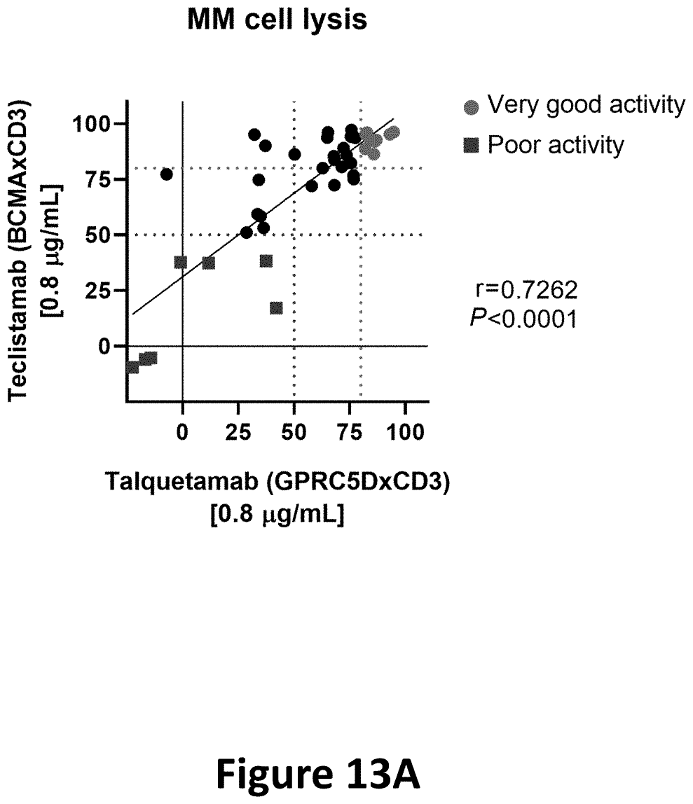

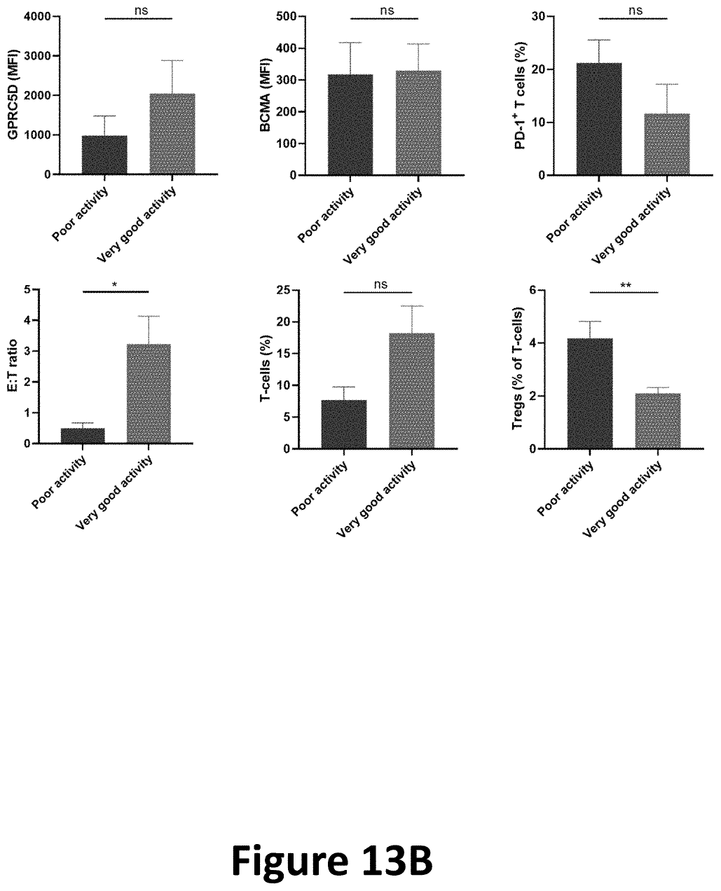

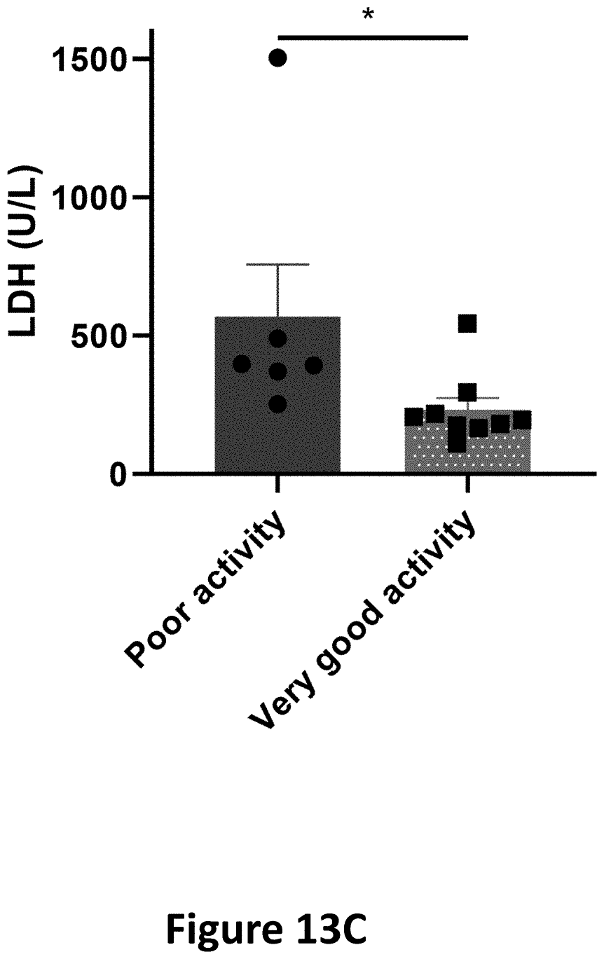

Since more T-cell redirecting bispecific antibodies targeting different tumor-associated antigens are being investigated in the field, a question was if there were shared determinants of response between bispecifics. A simultaneous evaluation of the single agent activity in 41 BM samples of both talquetamab and the BCMA-targeting bispecific antibody teclistamab (only differing in tumor-binding domain) was performed (FIGS. 13A-13C). MM cell lysis induced by both agents was strongly correlated. In 7 samples, both agents exhibited poor activity (<50% lysis), whereas in 9 samples very good activity was observed (>80% lysis). Comparison of characteristics between these groups showed that a low E:T ratio and high frequency of Tregs significantly impaired efficacy of both BsAbs (FIG. 13B). It was also shown that LDH-levels were significantly higher in patients who exhibited poor bispecific antibody activity (FIG. 13C). Altogether, it is suggested that patient-specific factors can determine response to T-cell redirectors targeting different antigens.

In summary, GPRC5D is a promising target for immunotherapeutic strategies and talquetamab showed marked ex vivo anti-MM activity, irrespective of disease stage or cytogenetic risk. Tumor-related factors (GPRC5D expression) and differences in the composition of the BM microenvironment (including E:T ratio and % of Tregs) were observed, contribute to the variability in response to talquetamab. The data also indicate that strategies aiming at optimizing E:T ratio (e.g. induction therapy) or Treg depletion, can improve response to bispecific antibodies in MM.

### Example 2: Nonclinical Pharmacology Studies of Talquetamab

Bispecific antibody talquetamab was generated by controlled fragment antigen binding arm exchange from two parental antibodies; GC5B596, an anti-GPRC5D antibody that originated from mouse immunization using the human GPRC5D DNA and GPRC5D overexpressing rat basophilic leukemia cells; and CD3B219, an anti-CD3F antibody that originated from a public domain antibody, SP34, which was further humanized and affinity matured. Talquetamab binds to human and cynomolgus CD3 and GPRC5D, and to rodent GPRC5D, but not to rodent CD3 (see e.g., U.S. Pat. No. 10,562,968).

### Talquetamab Binding to Multiple Myeloma Cell Lines

Talquetamab binds specifically to endogenous GPRC5D-expressing multiple myeloma cell lines in a dose-dependent manner, as measured by flow cytometry for all GPRC5D-positive cell lines that were tested (H929, MM.1R, and OPM-2). In contrast, Talquetamab did not bind to GPRC5D-negative cell lines, NALM-6 and Daudi cells.

### Talquetamab-Mediated T Cell Dependent Cytotoxicity of GPRC5D-Positive Cell Lines In Vitro

The T cell-dependent killing potential of talquetamab in multiple myeloma cells was determined in a flow cytometry-based cytotoxicity assay. Increasing concentrations of talquetamab were incubated with pan T cells from 6 healthy donors, 3 GPRC5D-positive and 2 GPRC5D-negative cell lines, at an effector:target (E:T) ratio of 5:1. These findings revealed that talquetamab induced cell death in GPRC5D-positive cell lines after 48 hours with average half maximal effective concentration (EC₅₀) (or 20% maximal effective concentration—EC₂₀) values for H929, MM.1R, OPM-2 of 0.057 (0.029), 0.015 (0.007) and 0.214 (0.091) nM, respectively. No cell lysis was observed in the negative control (GPRC5D-negative) cell lines or with control antibodies bearing an unrelated arm (null) paired with GPRC5D or CD3. To assess the T cell activation potential of talquetamab, cells were harvested from this assay after 48 hours of incubation and analyzed by flow cytometry for the expression of the T cell activation marker (CD25). Talquetamab (but not the negative control null molecules) induced potent T cell activation with average EC₅₀ (EC₂₀) values for H929, MM.1R, OPM-2 of 0.082 (0.035), 0.014 (0.006), and 0.288 (0.168) nM, respectively, when incubated with GPRC5D-positive multiple myeloma cells and healthy donor pan T cells. This was not the case in the 2 negative control cells (NALM-6 and Daudi). In vitro cytokine release was assessed in the supernatant from T cell-mediated killing assay (using T cells from 6 healthy donors) with H929 cells. The observed values for the average EC₅₀ (EC₂₀) were as follows: interferon (IFN)-γ: 1.120 (0.615) pg/mL; tumor necrosis factor (TNF)-α: 1.545 (0.805) pg/mL; interleukin (IL)-1β: 0.720 (0.462) pg/mL; IL-2: 1.962 (1.380) pg/mL; IL-4: 1.867 (1.733) pg/mL; IL-6: 0.684 (0.441) pg/mL; IL-8: 0.440 (0.273) pg/mL; IL-10: 1.082 (0.670) pg/mL. Talquetamab did not cause significant activation of T cells in the absence of target GPRCSD-positive cells in in vitro or in whole blood assay. These findings demonstrate the specificity of talquetamab. The effect of talquetamab on cytotoxicity, T cell activation, and cytokine release was also tested in an in vitro assay using whole blood from healthy donors. Whole blood was incubated with GPRC5D-positive (H929) multiple myeloma cells at an E:T ratio of 5:1 with increasing concentrations of talquetamab for 48 hours. Talquetamab-induced cell death in GPRC5D-positive cell lines after 48 hours. Mean EC₅₀ (EC₂₀) values for healthy donors were as follows: cytotoxicity 0.389 (0.131) nM, cytokine IL-10 0.107 (0.032) nM, and T cell activation 0.236 (0.083) nM.

### Talquetamab-Mediated T Cell Dependent Cytotoxicity of Primary Multiple Myeloma Cell Samples

The cytotoxicity effect of talquetamab was also evaluated in an ex vivo assay using frozen bone marrow (mononuclear cells) samples from patients with multiple myeloma (n=6) and T cells from healthy donors (at 1:1 ratio). The results revealed that talquetamab promoted a dose-dependent reduction of GPRC5D-positive primary multiple myeloma cells, which correlated with T cell activation after 48 hours. Average EC₅₀ (EC₂₀) values were as follows: cytotoxicity 0.127 (0.041) nM; T cell activation 0.061 (0.016) nM. The control null antibodies had no effect on cytotoxicity or T cell activation, suggesting that the induced cell-death effect is specific for talquetamab.

### Concentration Dependency of Talquetamab on Killing and T Cell Activation

To assess whether high concentrations of talquetamab can lead to epitope saturation on the target cells or T cells and inhibit synapse formation, T cell-mediated cytotoxicity assays were performed using H929 cells with increased concentrations of talquetamab up to 532 nM. Talquetamab showed a dose-dependent cytotoxicity and T cell activation up to the top concentration of 532 nM and no epitope saturation effect was observed.

### Effects of Talquetamab in Multiple Myeloma Xenograft Models In Vivo

Efficacy of GPRC5D×CD3 bispecific antibody talquetamab was evaluated in 3 GPRC5D-positive human multiple myeloma models in peripheral blood mononuclear cell (PBMC)-humanized NOD scid gamma (NSG, NOD.Cg-Prkdcscid) mice. Two models were used: a prophylactic model in which treatment was initiated at the time of tumor cell implantation (H929), or an established model in which treatment was initiated after palpable tumors were formed (MM.1S and RPMI 8226).

For the H929 prophylactic multiple myeloma model, mice were engrafted with 10 million PBMCs one week prior to tumor inoculation with 5×10⁶ H929 cells subcutaneous. Treatment with talquetamab at 0.1, 1, or 10 μg per mouse (corresponding to 0.005, 0.05, or 0.5 mg/kg) was initiated immediately and repeated every 3 to 4 days thereafter for a total of 5 doses. Talquetamab elicited complete blockade of tumor formation at dose levels of either 10 or 1 μg/mouse, and 0.1 μg/mouse either blocked tumor formation or significantly suppressed growth as compared with phosphate-buffered saline (PBS)-treated control mice (97.6% mean tumor growth inhibition as compared with control mice, p<0.01).

For the established MM.1S multiple myeloma model, NSG mice were inoculated with 1×10⁷ cells subcutaneously. One week later 10 million PBMCs were engrafted. Two weeks after tumor cell implantation, treatment with either GPCR5D×CD3 bispecific antibody talquetamab (0.1, 1, 10, or 50 μg per mouse), CD3×null, or GPRC5D×null (10 μg per mouse) was initiated and repeated every 3 to 4 days thereafter for a total of 7 doses. Antitumor efficacy was observed with 10 and 50 μg/animal dose levels of talquetamab bispecific antibody, with 10 out of the 10 complete responses (CR) (100% tumor regressions) in each group. Moreover, the 1 ag per mouse dose significantly inhibited tumor growth by 65% as compared with PBS-treated control animals (p≤0.05), whereas the CD3 x null bispecific antibody nor the GPRC5D×null bispecific antibody failed to suppress tumor growth in the model.

A separate regression study using RPMI 8226 target cells expressing minimal GPRC5D protein and human purified T cells from a healthy donor as effector cells showed that talquetamab had no effect on the tumor regression. GPRC5D was present at low levels. Additional studies on preclinical activity and determinants of response of talquetamab in MM are described in Verkleij et al., Blood Advances, 2021, 5(8): 2196-2215, the content of which is incorporated herein by reference in its entirety.

### Example 3: Toxicology and Safety Pharmacology

### Toxicology

Talquetamab was administered IV once weekly for 4 weeks in a non-good laboratory practice (GLP) tolerability study in cynomolgus monkeys and was well tolerated up to 30 mg/kg. There were no talquetamab related clinical signs, significant pharmacodynamic effects (e.g., cytokine release) or adverse effects on safety parameters. Other notable changes were non-adverse and generally consistent with the expected mechanism of action of talquetamab and included transient and mild decreases in lymphocyte counts. It was determined that talquetamab had an approximately 100-fold lower in vivo pharmacological activity to GPRC5D in cynomolgus monkeys compared with humans. Based on the poor cross-reactivity, lack of adverse effects, and minimal pharmacodynamic effects observed in this study, further nonclinical safety studies with talquetamab in the cynomolgus monkey were not considered useful for human risk assessment.

Therefore, hazard identification studies in cynomolgus monkey were conducted with the surrogate molecule, talquetamab, which had cross-reactivity to cGPRC5D, and its functional activity in cynomolgus monkey cells was similar to the activity of talquetamab in human cells and was considered pharmacologically relevant in cynomolgus monkey. In an exploratory 2-week tolerability study (2 weekly doses of 0, 0.3, 3, and 10 mg/kg) and a pivotal 1-month GLP study that included safety pharmacology assessments (4 weekly doses of 0, 10, and 30 mg/kg), talquetamab was well tolerated and no effects were noted on safety pharmacology parameters (cardiovascular, respiratory, or central nervous system function).

Overall, there was no robust pharmacodynamic or toxicological response in cynomolgus monkey with the study drug (talquetamab) that is typically expected from this class of molecule at the high doses tested. The minimal pharmacodynamics response with the surrogate molecule is potentially due to low target expression or low target cell (plasma cells) numbers in healthy cynomolgus monkeys. Hence, the results highlight the limitations of the cynomolgus monkey to provide useful information about risk assessment associated with targeting GPRC5D in patients with multiple myeloma (i.e., limited translatability of these nonclinical toxicity findings to patient who may carry a greater target cell burden).

### Local Tolerance

In a local tolerance study, a single subcutaneous (SC) injection of talquetamab (20 mg) was well tolerated in male New Zealand White rabbits. There were no adverse dermal observations at the injection sites (evaluated up to 72 hours post-dose), and no gross or microscopic findings at the injection sites or in draining lymph nodes. As the rabbit is not a pharmacologically relevant species for targeting of GPRC5D by talquetamab, this study only assessed the tolerability of the formulation.

### Tissue Cross-Reactivity, Serum, Hemolytic, and Cytokine Potential (In Vitro Studies)

An in vitro GLP tissue cross-reactivity study in human tissues showed biotinylated talquetamab staining on the membrane and cytoplasm of mononuclear leukocytes (likely due to binding to CD3) in human lymphoid tissues (lymph node, spleen, thymus, tonsil, bronchus-associated lymphoid tissue in the lung, and gut-associated lymphoid tissue in the gastrointestinal tract), and select non-lymphoid tissues (mononuclear cells in bladder, breast, colon, fallopian tube, kidney, liver, ovary, parathyroid, peripheral nerve, pituitary, placenta, prostate, salivary gland, thyroid, ureter, and uterus (cervix, endometrium)). No unanticipated cross-reactivity was observed. The ability of talquetamab to induce cytokine release in human donor blood was assessed using the soluble phase assay format assay. A solid phase assay format was also tested. In the soluble phase format, talquetamab induced statistically significant, dose-dependent increases in IL-1β, IL-2, IL-6, IL-8, IL-10, IL-13, IFNγ, and TNFα, relative to control.

Estimates of population EC₂0 values of talquetamab were: 346.39 nM for IL-1β, 195.49 nM for IL-2, 519.20 nM for IL-8, 6616.99 nM for IFNγ, and 859.97 nM for TNFα. EC₂₀ values could not be calculated for IL-6, IL-10, and IL-13 due to lack of a 4-parameter curve fit. In the solid phase format assay relative to control, talquetamab induced statistically significant release in 9 of the 10 cytokines (IL-1β, IL-2, IL-4, IL-6, IL-8, IL-10, IL-13, IFNγ, and TNFα). However, population EC₂₀ estimates could not be calculated for any of the cytokines induced due to lack of a 4-parameter curve fit. Because of the nature of response (e.g., biphasic response, poor curve fitting, and similar profile to the negative control), the biological relevance of the solid phase assay is unclear. Talquetamab did not cause hemolysis in whole human blood and was compatible with human serum at concentrations between 0.10 and 10 mg/mL.

In cynomolgus monkeys, systemic talquetamab exposure (maximum observed serum concentration [Cₘₐₓ] and area under the serum concentration versus time curve [AUC]) increased with dose in an approximately dose-proportional manner following IV administration of talquetamab as a single dose of 0.5 and 5 mg/kg in an rHSA formulation or 0.5 mg/kg in formulation buffer in a pharmacokinetic study. Talquetamab exposure also increased with dose in an approximately dose-proportional manner following weekly doses of 0.5 to 30 mg/kg in an exploratory 4-week tolerability study. The serum half-life of talquetamab was estimated at 9 to 12 days in cynomolgus monkeys.

### Example 4: Nonclinical Immunogenicity

A preliminary evaluation on immunogenicity risk for talquetamab was conducted. Given the similarity of talquetamab to native human monoclonal antibodies, the risk of immunogenicity is expected to be low to moderate, similar to other human therapeutic monoclonal antibodies. In the non-GLP single-dose pharmacokinetic study of talquetamab, immunogenicity results showed that 8 out of the 12 animals treated with talquetamab tested positive for anti-drug antibody (ADA). In the non-GLP multiple-dose studies of talquetamab, data suggest the presence of ADA (2 out of the 12 animals). However, ADA was not monitored in either of these studies. When compared to the ADA-negative animals in the same dose group, all ADA-positive animals exhibited either lower drug exposure prior to the last dose on Day 22 or faster concentration decrease after the dose.

### Example 5: Phase 1 Study of Talquetamab Administered as Monotherapy for Relapsed or Refractory Multiple Myeloma

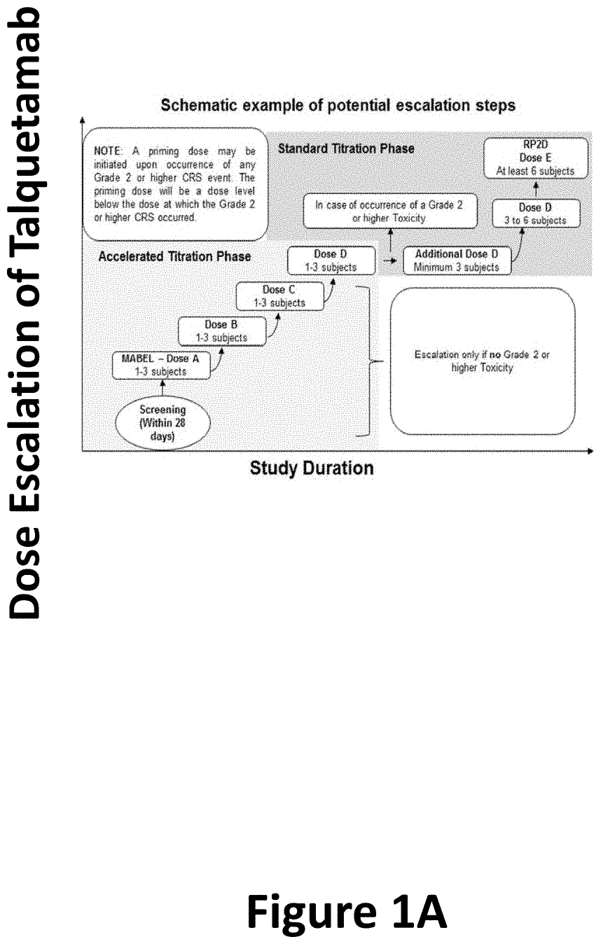

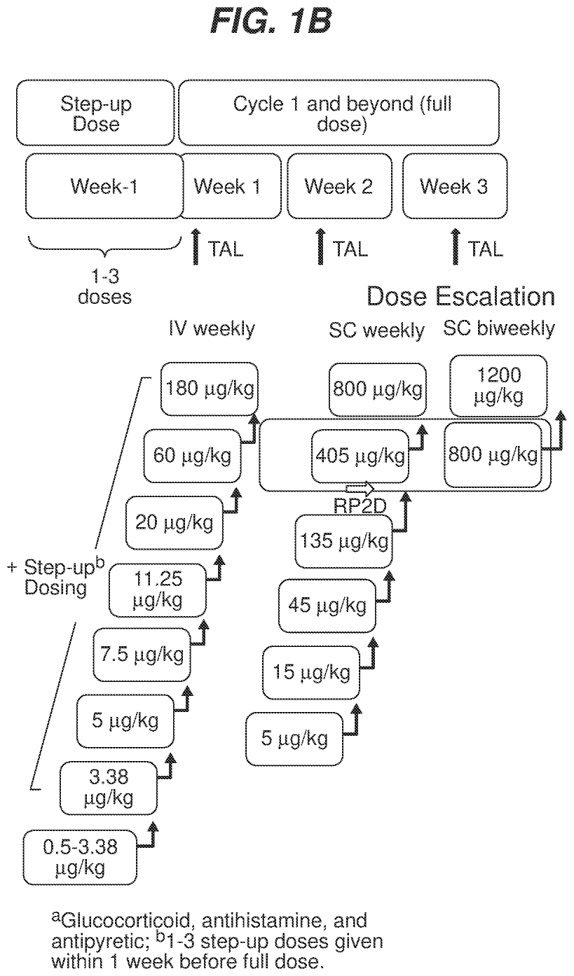

A first-in-human (FIH), phase 1, open-label, multicenter study of talquetamab administered to adult subjects with relapsed or refractory multiple myeloma was carried out (NCT03399799). The study was conducted in 2 parts, separately for IV and SC administration: dose escalation (Part 1) and dose expansion (Part 2). The overall aim of the study was to evaluate the safety of talquetamab. Safety was monitored by a Study Evaluation Team (SET). A diagram of the dose escalation scheme is provided in FIG. 1.

### Subject Population

The inclusion and exclusion criteria for enrolling subjects in this study are described below.

### Inclusion Criteria

≥18 years of age.
Documented initial diagnosis of multiple myeloma according to International
Myeloma Working Group (IMWG) diagnostic criteria.
Subjects with measurable multiple myeloma who have progressed on, or could not tolerate, all available established therapies.
Eastern Cooperative Oncology Group (ECOG) performance status score of 0 or 1.
Clinical laboratory values at screening: PGP-30JI1

Hematology

| | |
|---|---|
| Hemoglobin | ≥8.0 g/dL (≥5 mmol/L) (must be without red blood cell |
| | [RBC] transfusion within 7 days prior to the laboratory |
| | test; recombinant human erythropoietin use is permitted). |
| Platelets | ≥50 × 10⁹/L (must be without transfusion support or |
| | platelet stimulating factor in the 7 days prior |
| | to the laboratory test) |
| Absolute | ≥1.0 × 10⁹/L (prior growth factor support is permitted |
| Neutrophil | but must be without support in the 7 days prior |
| Count (ANC) | to the laboratory test) |

Chemistry

| | |
|---|---|
| Aspartate | ≤3.0 × upper limit of normal (ULN) |
| Amino- | |
| transferase | |
| or Alanine | |
| Amino- | |
| transferase | |
| Creatine | ≥40 mL/min/1.73 m² based upon Modified Diet in |
| | Renal Disease formula calculation. |
| Total bilirubin | <2.0 × ULN; except in subjects with congenital |
| | bilirubinemia, such as Gilbert syndrome (in which case |
| | direct bilirubin ≤1.5 × ULN is required) |
| Corrected | ≤14 mg/dL (≤3.5 mmol/L) or free ionized calcium <6.5 |
| serum calcium | mg/dL (<1.6 mmol/L). |

Women of childbearing potential (WOCBP) must have a negative pregnancy test at screening and prior to the first dose of study drug using a highly sensitive pregnancy test either serum (β human chorionic gonadotropin [β-hCG]) or urine. Before the first dose of study drug: Women of childbearing potential and fertile men who are sexually active must agree to use a highly effective method of contraception (<100/year failure rate) during the study and for 100 days after the last dose of study drug. Sign an informed consent form (ICF) indicating that he or she understands the purpose of and procedures required for the study and is willing to and able participate in the study. Consent is to be obtained prior to the initiation of any study-related tests or procedures that are not part of standard of care for the subject's disease.

Willing and able to adhere to the prohibitions and restrictions specified in this protocol.

### Exclusion Criteria

Any potential subject who meets any of the following criteria will be excluded from participating in the study:
Prior Grade 3 or higher CRS related to any T cell redirection (e.g., CD-3 redirection technology or CAR-T cell therapy) or any prior GPRC5D targeting therapy.
Prior antitumor therapy as follows, prior to the first dose of study drug:
Gene modified adoptive cell therapy (e.g., chimeric antigen receptor modified T cells, natural killer [NK] cells) within 3 months.
Targeted therapy, epigenetic therapy, or treatment with an investigational drug or an invasive investigational medical device within 21 days or at least 5 half-lives, whichever is less.
Monoclonal antibody treatment for multiple myeloma within 21 days.
Cytotoxic therapy within 21 days.
Proteasome inhibitor therapy within 14 days.
Immunomodulatory agent therapy within 7 days.
Radiotherapy within 21 days. However, if the radiation portal covered 5% of the bone marrow reserve, the subject is eligible irrespective of the end date of radiotherapy.
Vaccinated with live, attenuated vaccine within 4 weeks or as recommended by the product manufacturer prior to the first dose, during treatment, or within 100 days of the last dose of talquetamab.
Toxicities from previous anticancer therapies should have resolved to baseline levels or to Grade 1 or less except for alopecia or peripheral neuropathy.
Received a cumulative dose of corticosteroids equivalent to ≥140 mg of prednisone within the 14-day period before the first dose of study drug.
Received either of the following:
   An allogenic stem cell transplant within 6 months before first dose of study drug.
   Subjects who received an allogeneic transplant must be off all immunosuppressive medications for 6 weeks without signs of GVHD.
   An autologous stem cell transplant 12 weeks before first dose of study drug.
Central nervous system (CNS) involvement or clinical signs of meningeal involvement of multiple myeloma. If either is suspected, negative whole brain magnetic resonance imaging (MRI) and lumbar cytology are required.
Plasma cell leukemia (>2.0×10⁹/L plasma cells by standard differential), Waldenstrom's macroglobulinemia, POEMS syndrome (polyneuropathy, organomegaly, endocrinopathy, monoclonal protein [M-protein], and skin changes), or primary amyloid light chain (AL) amyloidosis.
Known to be seropositive for human immunodeficiency virus or acquired immune deficiency syndrome.
Hepatitis B infection as defined according to the American Society of Clinical Oncology guidelines. In the event the infection status is unclear, quantitative levels are necessary to determine the infection status. Active Hepatitis C infection as measured by positive HCV-RNA testing. Subjects with a history of Hepatitis C virus antibody positivity must undergo HCV-RNA testing.
Pulmonary compromise requiring supplemental oxygen use to maintain adequate oxygenation.
Known allergies, hypersensitivity, or intolerance to talquetamab or its excipients.
Any serious underlying medical condition, such as:
Evidence of serious active viral, bacterial, or uncontrolled systemic fungal infection;
Active autoimmune disease or a documented history of autoimmune disease
Psychiatric conditions (e.g., alcohol or drug abuse), severe dementia, or altered mental status; and
Any other issue that would impair the ability of the subject to receive or tolerate the planned treatment at the investigational site, to understand informed consent or any condition for which, in the opinion of the investigator, participation would not be in the best interest of the subject (e.g., compromise the well-being) or that could prevent, limit, or confound the protocol-specified assessments.
Pregnant, breastfeeding, or planning to become pregnant while enrolled in this study or within 100 days after the last dose of study drug.
Plans to father a child while enrolled in this study or within 100 days after the last dose of study drug.
Major surgery within 2 weeks of the first dose, or will not have fully recovered from surgery, or has surgery planned during the time the subject is expected to participate in the study or within 2 weeks after the last dose of study drug administration. (Note: subjects with planned surgical procedures to be conducted under local anesthesia may participate).

The subjects must agree to not donate blood or blood components during the study and for 100 days after the last doses of study drug.

NOTE: All study inclusion/exclusion criteria were ensured to have been met at screening and prior to the first dose of study drug. If a subject's clinical status changes (including any available laboratory results or receipt of additional medical records) after screening but before the first dose of study drug was given such that he or she no longer meets all eligibility criteria, supportive treatment might be administered according to local standards of care, if necessary, so that eligibility criteria might be met and laboratory test(s) might be repeated once, to determine if the subject qualified for the study. If inclusion/exclusion criteria were not met after further evaluation, the subject should be excluded from participation in the study.

### Pharmacokinetics, Immunogenicity, and Receptor Occupancy

### Evaluations

Blood and serum samples were collected for talquetamab pharmacokinetics, receptor occupancy (RO), and immunogenicity (antibodies to talquetamab assessment). Also, a pharmacokinetic/immunogenicity sample would be collected any time a suspected infusion-related reaction (IRR) or cytokine release syndrome (CRS) event (in case of a CRS event, samples will be collected at onset, 24 hours, and 72 hours) was observed during the study. In addition, pharmacokinetic and immunogenicity samples were collected at the End-of-Treatment visit following study drug discontinuation. The exact dates and times of blood sampling were recorded on the laboratory requisition forms. Collected samples were stored under specified controlled conditions for the temperatures indicated in the Laboratory Manual.

Venous blood samples were collected for measurement of serum concentrations of talquetamab. The serum sample would be evenly divided into 2 aliquots (1 for pharmacokinetics; 1 for backup). At timepoints where serum concentration and antibodies to talquetamab would be evaluated, 1 blood draw was collected and the serum was evenly divided into 3 aliquots (1 each for pharmacokinetics, antibodies to study drug, and a backup).

Blood samples were collected for RO. Bone marrow aspirate might also be analyzed for pharmacokinetics, if feasible. Data were used for mechanistic pharmacokinetic/pharmacodynamic modeling. Samples collected for analyses of talquetamab serum concentration and antibody to talquetamab might be used to evaluate soluble B cell maturation antigen (sBCMA) or to evaluate safety or efficacy aspects that address concerns arising during or after the study period for further characterization of immunogenicity.

### Analytical Procedures

Pharmacokinetics: Serum samples were analyzed to determine concentrations of talquetamab using a validated, specific, and sensitive assay method by or under the supervision of the sponsor.

Immunogenicity: The detection and characterization of anti-talquetamab antibodies were performed using a validated or appropriately qualified assay methods by or under the supervision of the sponsor. All samples collected for detection of antibodies to talquetamab would also be evaluated for talquetamab serum concentration to enable interpretation of the antibody data.

Pharmacokinetic Parameters Blood samples were collected from all subjects for the measurement of serum talquetamab concentration for pharmacokinetic analyses. Pharmacokinetic parameters were estimated for individuals, and descriptive statistics were calculated for each dose level. Cₘₐₓ and AUC with dose might also be explored. Pharmacokinetic parameters include, but were not limited to, AUC_{inf}, AUC₍₀₋ₜ₎, AUCₜₐᵤ, Cₘₐₓ, T_{1/2}, time to reach the Cₘₐₓ (Tₘₐₓ), CL (for IV administration), CL/F (for SC administration), volume of distribution at steady-state ([Vss] for IV administration); and Vss/F (for SC administration) parameters were calculated if sufficient data were available for estimation. (AUC: area under the serum concentration versus time curve; AUC_{inf}: area under the serum concentration versus time curve from time 0 to infinity with extrapolation of the terminal phase; AUC₍₀₋ₜ₎: area under the concentration-time curve from time zero to time t; AUCₜₐᵤ: area under the serum concentration versus time curve during a dose interval time period (tau) at steady-state; Cₘₐₓ: maximum observed serum concentration; T_{1/2}: half-life; Tₘₐₓ: time to reach the Cₘₐₓ (multiple doses); CL: total systemic clearance of drug after intravenous administration; Vss: volume of distribution at steady-state).

### Immunogenicity Assessments Antibodies to Talquetamab

Antibodies to talquetamab were evaluated in serum samples collected from all subjects according to schedules. Additionally, serum samples were also collected at the final visit from subjects who were discontinued from treatment or withdrawal from the study. These samples were tested by the sponsor or sponsor's designee.

Serum samples were screened for antibodies binding to talquetamab and the titer of confirmed positive samples were reported. The ADA-positive (anti-drug antibody-positive) samples were tested for neutralizing antibodies to talquetamab. Immune response analysis might be conducted on pharmacokinetic samples collected at other timepoints, if deemed necessary.

### Receptor Occupancy (RO)

Whole blood samples were analyzed for RO via flow cytometry. Samples were collected to evaluate RO to quantify the binding of therapeutics to CD3 on the cell surface. RO samples were collected in Part 2 if the RO sample results from Part 1 were comprehensive and meaningful for drug response. Sample was collected from all subjects, based on emerging data, prior to the first intravenous (IV) priming dose and for some subjects treated with higher subcutaneous (SC) dosing.

### Biomarker Evaluations

Biomarker evaluations were completed in both Part 1 and Part 2. The biomarker assessments focused on several main objectives: 1) evaluate cytokine production in response to study drug administration; 2) evaluate the immune responses indicative of T cell redirection for potential contributions to study drug response; 3) determine the clinical benefit of study drug in subjects with cytogenetic modifications (del17p, t(4;14), t(14;16), or other high-risk molecular subtypes); and 4) determine the ability of study drug to reduce minimal residual disease (MRD) in subjects who had at least a complete response (CR). All biomarker assessments were performed at a central laboratory. If it became necessary, additional biomarker samples might be collected to help understand an unexplained event and specifically additional sample(s) for cytokines would be collected any time a suspected IRR or CRS event was observed or reported during the study.

Biomarker analyses were dependent upon the availability of appropriate biomarker assays and might be deferred or not performed, if during or at the end of the study, it became clear that the analysis would not have sufficient scientific value for biomarker evaluation, or if there were not enough samples or responders to allow for adequate biomarker evaluation. In the event the study was terminated early or showed poor preliminary clinical antitumor activity, completion of biomarker assessments was based on justification and intended utility of the data.

### Additional Collections

Based on emerging scientific evidence, the sponsor might request additional material from previously collected bone marrow samples during or after study completion for a retrospective analysis. In this case, such analyses would be specific to research related to the study drug(s) or diseases being investigated.

### Pharmacodynamic Markers

Serum samples were collected before and at multiple time points after talquetamab administration at step-up or full treatment doses as scheduled. Cytokine were detected and measured using multi-plexed analyte panels (Luminex or MEsoScaleDiscovery technology). Analyses monitored included, but were not limited to TNF-α, IL-2, IL-6, INF-γ, IL-10, and IL-2Rα, which can inform on relative activation of immune cells.

Whole blood samples and bone marrow aspirate samples might be analyzed to evaluate tumor and immune cell populations by flow cytometry and/or cytometry by time of flight (CyTOF) in order to determine if treatment with talquetamab results in increased antitumor activity by redirected T cell-mediated killing of GPRC5D-positive multiple myeloma cells and increased activation of cytotoxic T cells. Whole blood T cell functionality assays might also be performed to study how this could affect drug response.

For these analyses, whole blood samples collected pre- and post-talquetamab administration were analyzed using multi-color flow cytometry to assess immune populations, including, but not limited to, CD8+, CD4+ total and regulatory T cells, as well as naïve and memory T cell subsets. In addition, activation/exhaustion markers including CD25, PD-1, TIM-3, LAG-3, HLA-DR and CD38 were also measured on CD8+ and CD4+ total and naïve/memory T cell sub.

### Predictive Biomarkers

Some genetic mutations/translocations are known to confer a poor prognosis in multiple myeloma therapy and resistance. Therefore, DNA/RNA sequencing from tumor cells might be performed, for translocation/mutation/genomic analysis to assess whether specific molecular subgroups such as del17p, t(4;14), t(14;16) or other risk associated mutations/translocations were responsive to treatment and to identify potential predictive biomarkers of response and/or resistance.

GPRC5D and PD-LI expression on plasma cells at baseline may also be measured by flow cytometry on multiple myeloma cells in bone marrow samples to determine if antigen expression level or checkpoint ligand upregulation is a predictive biomarker of response.

Baseline immunophenotyping, including, but not limited to, frequency and activation/exhaustion of T cell subsets may also be performed on bone marrow aspirates to determine potential predictive biomarkers of response and/or resistance.

### Minimal Residual Disease

Minimal residual disease negativity was being evaluated in the field as a potential surrogate for progression-free survival (PFS). Baseline bone marrow aspirates will be used to define the myeloma clones, and posttreatment samples would be used to evaluate MRD negativity in those subjects who experience a CR/stringent complete response (sCR). A fresh bone marrow aspirate was to be collected at screening, where clinically feasible. If bone marrow aspirate was not available at screening, non-decalcified diagnostic tissue, such as non-decalcified slides (bone marrow aspirate, touch preparation or clot selection) or formalin-fixed, paraffin-embedded block (clot section only, no bone marrow biopsy), must be supplied for MRD assessment instead. Minimal residual disease would be monitored in subjects using next generation sequencing on bone marrow aspirate DNA. If this methodology is unavailable, or determined to be scientifically inferior, then alternative methods for MRD assessment might be utilized.

### Pharmacokinetic/Pharmacodynamic Evaluations

Pharmacokinetic/pharmacodynamic modeling was explored to understand and characterize the exposure-response relationship.

### Efficacy Evaluations

Disease evaluations were performed at the end of each treatment cycle and prior to the start of the next cycle. Disease evaluations scheduled for treatment days should be collected before study drug is administered. Disease evaluations would be performed by a central laboratory until disease progression. This study would use the 2016 IMWG-based response criteria. If it was determined that the study drug interferes with the immunofixation assay, CR would be defined as the disappearance of the original M-protein associated with multiple myeloma on immunofixation, and the determination of CR would not be affected by unrelated M-proteins secondary to the study drug. Subjects who relapse should not be taken off treatment and disease evaluations would continue until disease progression is confirmed.

Disease progression must be consistently documented across clinical study sites. Note: the onset of new or increased size of soft tissue plasmacytomas and lytic bone lesions observed during Cycle 1 would not be considered disease progression. The sponsor would use a validated computer algorithm to assess response to treatment.

### Myeloma Protein Measurements in Serum and Urine

Blood and 24-hour urine samples for M-protein measurements were analyzed by the central laboratory. The following tests were required:
Serum protein electrophoresis;
Serum immunofixation electrophoresis at screening and thereafter when M-protein is non-quantifiable;
Serum FLC assay;
24-hour urine M-protein quantitation by electrophoresis;
Urine immunofixation electrophoresis at screening and thereafter when M-protein is non-quantifiable;
Serum β-microglobulin at screening; and
Serum quantitative immunoglobulins (IgG, IgA, IgM, IgE, and IgD) at screening.

Samples for serum quantitative immunoglobulins (IgG, IgA, IgM, IgE, and IgD) were also collected at screening and every 4 weeks thereafter to be analyzed locally. Blood and 24-hour urine samples were collected until the development of confirmed disease progression. Disease progression based on one of the laboratory tests alone were confirmed by at least 1 repeat investigation performed 1 to 3 weeks later. Disease evaluations would continue beyond relapse from CR until disease progression was confirmed. Serum and urine immunofixation and serum free light chain (FLC) assay would be performed at screening and thereafter when a CR was suspected (when serum or 24-hour urine M-protein electrophoresis [by serum protein electrophoresis or urine M-protein quantitation by electrophoresis (UPEP)] were 0 or non-quantifiable). Both serum and urine immunofixation test would be performed routinely for subjects with light chain multiple myeloma.

### Serum Calcium Corrected for Albumin

Blood samples for calculating serum calcium corrected for albumin were collected and analyzed at the central laboratory until the development of confirmed disease progression. Development of hypercalcemia (corrected serum calcium >11.5 mg/dL [>2.8 mmol/L]) could indicate disease progression or relapse if it was not attributable to any other cause. Calcium bound to albumin and only the unbound (free) calcium was biologically active; therefore, the serum calcium level must be adjusted for abnormal albumin levels (“corrected serum calcium”).

Measurement of free ionized calcium was an acceptable alternative to corrected serum calcium for determining hypercalcemia. Free ionized calcium levels greater than the upper limit of normal (ULN) are considered to be hypercalcemic for this study.

### Bone Marrow Examination

For all subjects, bone marrow aspirate or biopsy were performed for clinical assessments and biomarker evaluations. Clinical staging (morphology, cytogenetics, and immunohistochemistry or immunofluorescence or flow cytometry) might be done by a local laboratory. A bone marrow aspirate sample was required to confirm CR and sCR; the sample must be collected and the results obtained prior to the next scheduled dose of study drug. A bone marrow aspirate sample was also collected at Cycle 3 Day 1 and at the time of disease progression, if clinically indicated.

In addition, MRD might be evaluated at the time of suspected CR/sCR, and for subjects with confirmed CR/sCR, an additional bone marrow aspirate was obtained 12 months post C1D1 (±1 month) and yearly (±1 month) thereafter.

### Skeletal Survey

A complete skeletal survey (including skull, entire vertebral column, pelvis, chest, humeri, femora, and any other bones for which the investigator suspects involvement by disease) was to be performed during the screening phase and evaluated locally by either roentgenography or low dose CT scans without the use of IV contrast. During the treatment phase, and before disease progression was confirmed, X-rays, or CT scans would be performed locally, whenever clinically indicated based on symptoms, to document response or progression. Magnetic resonance imaging was an acceptable method for evaluation of bone disease and might be included at the discretion of the investigator; however, it would not replace the skeletal survey. If a radionuclide bone scan was used at screening in addition to the complete skeletal survey, then both methods must be used to document disease status. These tests must be performed at the same time. Note: a radionuclide bone scan would not replace a complete skeletal survey.

Subjects might present with disease progression manifested by symptoms of pain due to bone changes. Therefore, disease progression might be documented, in these cases, by skeletal survey or other radiographs, depending on the symptoms that the subject experiences. If the diagnosis of disease progression was obvious by radiographic investigations, then no confirmatory X-rays were necessary. In instances in which changes were subtler, a repeat X ray might be needed in 1 to 3 weeks.

### Documentation of Extramedullary Plasmacytomas

Sites of known extramedullary plasmacytomas must be documented during the screening phase. Clinical examination or MRI might be used to document extramedullary sites of disease. CT scan evaluations were an acceptable alternative if there is no contraindication to the use of IV contrast. Positron emission tomography (PET)-CT was allowed if CT alone was not available. Ultrasound tests were not acceptable to document the size of extramedullary plasmacytomas.

Extramedullary plasmacytomas were assessed for all subjects with a history of plasmacytomas or if clinically indicated at screening, by clinical examination or radiologic imaging. Assessment of measurable sites of extramedullary disease would be performed, measured, and evaluated locally every 4 weeks (for physical examination) for subjects with a history of plasmacytomas or as clinically indicated during treatment for other subjects until development of confirmed CR or confirmed disease progression. If assessment could only be performed radiologically, then evaluation of extramedullary plasmacytomas might be done every 12 weeks (+2 weeks). For every subject, the methodology used for evaluation of each disease site was consistent across all visits. Irradiated or excised lesions would be considered not measurable and would be monitored only for disease progression.

To qualify for partial response (PR) or minimal response (MR), the sum of products of the perpendicular diameters of the existing extramedullary plasmacytomas must have decreased by at least 50% or 25%, respectively, and new plasmacytomas must not have developed. To qualify for disease progression, either the sum of products of the perpendicular diameters of the existing extramedullary plasmacytomas must have increased by at least 50% or a new plasmacytoma must have developed, except in Cycle 1. In the cases where not all existing extramedullary plasmacytomas were reported, but the sum of products of the perpendicular diameters of the reported plasmacytomas have increased by at least 50%, this would also qualify as disease progression.

### Criteria for Response to Multiple Myeloma Treatment

| Response | Response Criteria |
|---|---|
| Stringent | CR as defined below, plus |
| complete | Normal FLC ratio, and |
| Response | Absence of clonal PCs by immunohistochemistry, |
| (sCR) | immunofluorescence or 2- to 4-color flow cytometry |
| Complete | Negative immunofixation on the serum and urine, and |
| response | Disappearance of any soft tissue plasmacytomas, and |
| (CR)* | <5% PCs in bone marrow |
| Very | Serum and urine M-component detectable by immunofixation |
| good | but not on electrophoresis, or |
| partial | ≥90% reduction in serum M-protein plus urine M-protein <100 |
| Response | mg/24 hours |
| (VGPR)* | |
| Partial | ≥50% reduction of serum M-protein and reduction in 24-hour |
| response | urinary M-protein by ≥90% or to <200 mg/24 hours |
| (PR) | If the serum and urine M-protein are not measurable, a decrease |
| | of ≥50% in the difference between involved and uninvolved FLC |
| | levels is required in place of the M-protein criteria |
| | If serum and urine M-protein are not measurable, and serum free |
| | light assay is also not measurable, ≥50% reduction in bone marrow |
| | PCs is required in place of M-protein, provided baseline bone |
| | marrow plasma cell percentage was ≥30% |
| | In addition to the above criteria, if present at baseline, a ≥50% |
| | reduction in the size of soft tissue plasmacytomas is also required. |
| Minimal | ≥25% but ≤49% reduction of serum M-protein, and |
| response | Reduction in 24-h urine M-protein by 50-89% |
| (MR) | In addition to the above criteria, if present at baseline, a 25% |
| | to 49% |
| | reduction in the size of soft tissue plasmacytomas also is required |
| Stable | Not meeting criteria for CR, VGPR, PR, MR, or PD |
| disease | |
| (SD) | |
| Pro- | Increase of 25% from lowest response value in any one of the |
| gressive | following: Serum M-component (absolute increase |
| disease | must be ≥0.5 g/dL) Urine M-component (absolute increase |
| (PD)† | must be ≥200 mg/24 hours) |
| | Only in subjects without measurable serum and urine M-protein |
| | levels: the difference between involved and uninvolved FLC levels |
| | (absolute increase must be >10 mg/dL) |
| | Only in subjects without measurable serum and urine M-protein |
| | levels and without measurable disease by FLC levels, bone |
| | marrow PC percentage (absolute percentage must be ≥10%) |
| | Definite development of new bone lesions or soft tissue |
| | plasmacytomas or definite increase in the size of existing bone |
| | lesions or soft tissue plasmacytomas |
| | Development of hypercalcemia (corrected serum calcium >11.5 |
| | mg/dL) that can be attributed solely to the PC proliferative |
| | disorder categories also require no known evidence of progressive |
| | or of the products of the cross-diameters of the measurable lesion |

CR = complete response;
FLC = free light chain;
IMWG = International Myeloma
Working Group; M-protein = monoclonal paraprotein;
MR = minimal response;
PC = plasma cell;
PD = progressive disease;
PR = partial response;
sCR = stringent complete response;
SD = stable disease;
VGPR = very good partial response
All response categories (CR, sCR, VGPR, PR, and PD) require 2 consecutive assessments made at any time before the institution of any new therapy; CR, sCR, VGPR, PR, and SD new bone lesions if radiographic studies were performed. VGPR and CR categories require serum and urine studies regardless of whether disease at baseline was measurable on serum, urine, both, or neither. Radiographic studies are not required to satisfy these response requirements. Bone marrow assessments need not be confirmed. For PD, serum M-component increases of more than or equal to 1 g/dL are sufficient to define relapse if starting M-component is ≥5 g/dL.
*Clarifications to IMWG criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a >90% decrease in the difference between involved and uninvolved FLC levels.
†Clarifications to IMWG criteria for coding PD: Bone marrow criteria for PD are to be used only in subjects without measurable disease by M-protein and by FLC levels; “25% increase” refers to M-protein, FLC, and bone marrow results, and does not refer to bone lesions, soft tissue plasmacytomas, or hypercalcemia and the “lowest response value” does not need to be a confirmed value.
^{a}Presence/absence of clonal cells is based upon the kappa/lambda ratio. An abnormal kappa/lambda ratio by immunohistochemistry or immunofluorescence requires a minimum of 100 plasma cells for analysis. An abnormal ratio reflecting presence of an abnormal clone is kappa/lambda of >4:1 or <1:2.
Clinical Relapse
Clinical relapse is defined using the definition of clinical relapse in IMWG criteria (Durie 2006; Kumar 2016, Rajkumar 2011). In IMWG criteria, clinical relapse is defined as requiring one or more of the following direct indicators of increasing disease or end-organ dysfunction that are considered related to the underlying plasma cell proliferative disorder:
1. Development of new soft tissue plasmacytomas or bone lesions on skeletal survey, MRI, or other imaging
2. Definite increase in the size of existing plasmacytomas or bone lesions. A definite increase is defined as a 50% (and at least 1 cm) increase as measured serially by the sum
3. Hypercalcemia (>11.5 mg/dL; >2.875mM/L)
4. Decrease in hemoglobin of more than 2 g/dL (1.25 mM) or to less than 10 g/dL
5. Rise in serum creatinine by more than or equal to 2 mg/dL (≥177 mM/L)
6. Hyperviscosity
In some subjects, bone pain may be the initial symptom of relapse in the absence of any of the above features. However, bone pain without imaging confirmation is not adequate to meet these criteria in studies.

### Objectives and Endpoints

| | |
|---|---|
| Objectives | Endpoints |

Primary

| | |
|---|---|
| Part 1 (Dose Escalation): To | Part 1 (Dose Escalation): Frequency |
| characterize the safety of talquetamab | andt ype of DLT, and frequency |
| and recommend the Phase 2 dose(s) | and severity of adverse events, |
| and schedule | serious adverse events, and |
| Part 2 (Dose Expansion): To further | laboratory abnormalities |
| characterize the safety of | Part 2 (Dose Expansion): Frequency |
| talquetamab at | and severity of adverse events, |
| the recommended Phase 2 dose(s) | serious adverse events, and |
| (RP2Ds) | laboratory abnormalities |
| To characterize the pharmacokinetics | Pharmacokinetic parameters and |
| and pharmacodynamics of | pharmacodynamic markers |
| talquetamab | including but |
| To assess the immunogenicity of | not limited to depletion of GPRC5D |
| talquetamab | expressing cells, systemic cytokine |
| To evaluate the preliminary antitumor | concentrations, and markers of T |
| activity of talquetamab at the | cell activation |
| RP2D(s) in Part 2 | Presence of anti-talquetamab |
| | antibodies Assess the ORR |
| | (at least a PR or better); |
| | CBR; DOR and TTR; |
| | and PFS, as defined by the IMWG |
| | response criteria |

Secondary
To explore the relationships between pharmacokinetics,
pharmacodynamics, adverse event profile, and clinical activity
of talquetamab.
To investigate predictive biomarkers of response or resistance to
talquetamab.
To investigate the immunoregulatory activity of talquetamab.
To quantify receptor occupancy (RO), when feasible.
To evaluate MRD negativity rates.
To evaluate the exposure-response relationship.
CBR: clinical benefit rate; DOR: duration of response; TTR: time to response; PFS: progression-free survival; MRD: minimal residual disease.

### Part 1 (Dose Escalation Part)—Dosing Schedule

IV administration: A whole blood in vitro assay system from healthy human donors was used to estimate the minimum anticipated biologic effect level (MABEL)-based starting dose. A dose of 0.5 μg/kg IV administered over approximately 4-hours once every 2 weeks was selected based on the lowest mean EC₂O from the most relevant assay among T cell activation, cytotoxicity, and cytokine release. Subsequent bi-weekly IV dose levels were selected based on the review of all available data including, but not limited to, pharmacokinetic, pharmacodynamic, safety, and preliminary antitumor activity data. Preliminary first dose pharmacokinetic results from 3 subjects (dose range 0.5 to 1.0 μg/kg) following bi-weekly IV dosing with talquetamab showed that T_{1/2} ranged from 2.12 to 6.47 days. Based on the safety profile and preliminary pharmacokinetic data, weekly IV dosing with talquetamab was initiated. Subsequent dose levels were selected based on a statistical model using all available data to identify safe and tolerable putative RP2D(s), defined as the dose(s) and schedule(s) of talquetamab for characterization in Part 2.

SC administration: talquetamab was administered subcutaneously (SC) on a weekly dosing schedule. Dose escalation for the SC dosing cohort began with a 1.5 g/kg priming dose administered SC on Day −7, followed by a full dose of 5 μg/kg administered SC on Days 1, 8, and 15 of a 21-day cycle. Subsequent SC dose levels were selected based on a statistical model using all available data to identify safe and tolerable putative RP2D3(s), defined as the dose(s) of talquetamab for characterization in Part 2.

The following dosage levels had been tested in 182 patients and the cut-off date for the analyses was Apr. 18, 2021:

| Bi-weekly IV | Weekly IV | Weekly SC | Bi-weekly SC |
|---|---|---|---|
| (N = 26) | (N = 76) | (N = 63) | (N = 22) |
| Cohort 1 (0.5 | Cohort 6 (2.25 | Cohort 12 (1.5 + | Cohort 24 |
| μg/kg) = 1 | μg/kg) = 12 | 5 μg/kg) = 4 | (10/60/300 + 800 |
| Cohort 2 (1.0 | Cohort 7 (1.5 | Cohort 15 (5 + | μg/kg) = 15 |
| μg/kg) = 2 | μg/kg) = 6 | 15 μg/kg) = 4 | Cohort 26 + 27 PT2 |
| Cohort 3 (1.5 | Cohort 8 | Cohort 16 (10 + | (10/60/300 + 800 |
| μg/kg) = 10 | (1.5 + 3.38 | 45 μg/kg) = 6 | μg/kg) = 8 |
| Cohort 4 (2.25 | μg/kg) = 12 | Cohort 17 | Cohort 28 |
| μg/kg) = 11 | Cohort 9 (1.5 + | (10/45 + 135 | (10/60/300 + 1200 |
| Cohort 5 (3.38 | 5 μg/kg) = 12 | μg/kg) = 5 | μg/kg) = 9 |
| μg/kg) = 2 | Cohort 10 | Cohort 18 | |
| | (1.5 + 7.5 | (10/45 + 135 | |
| | μg/kg) = 10 | μg/kg) = 3 | |
| | Cohort 11 | Cohort 20 | |
| | (1.5 + 11.25 | (10/60 + 405 | |
| | μg/kg) = 6 | μg/kg) = 12 | |
| | Cohort 13 | Cohort 22 | |
| | (1.5/1.5 + 20 | (10/60/300 + | |
| | μg/kg) = 3 | 800 μg/kg) = 11 | |
| | Cohort 14 | Cohort 23 PT 2 | |
| | (1.5/10 + 20 | (10/60 + 405 | |
| | μg/kg) = 6 | μg/kg) = 18 | |
| | Cohort 19 | | |
| | (1.5/10 + 60 | | |
| | μg/kg) = 6 | | |
| | Cohort 21 | | |
| | (1.5/10/60 + | | |
| | 180 μg/kg) = 3 | | |

See also FIG. 1B for the study design.

### Results of Part 1 (Dose Escalation) (Cut-Off Date for Analyses Oct. 24, 2020)

Eligible patients have measurable MNM per International Myeloma Working Group (IN4WG) criteria and have progressed on or could not tolerate established therapies. The primary objectives of the dose escalation phase are to characterize the safety of talquetamab and to identify a recommended phase 2 dose (RP2D). Escalating doses of IV or SC talquetamab (0.5-800 μg/kg) with and without step-up dosing were assessed. Key secondary objectives include characterizing the pharmacokinetics (PK), pharmacodynamics, and preliminary antitumor activity of talquetamab. Adverse events (AEs) were graded using the Common Terminology Criteria for AE, v4.03, and cytokine release syndrome (CRS) was graded according to Lee et al (Blood 2014; 124:188).

Response was assessed by the investigator according to IMWG criteria. Among the subjects evaluated, 102 received talquetamab by IV and 29 received talquetamab by SC. Median age was 65 years (range 33-80; 32% were ≥70) and 23% had International Staging System stage III disease. Median number of prior therapies was 6 (range 2-20), 87% of subjects were refractory to last line of therapy, 80% triple-class refractory, 75% penta-class exposed, and 33% penta-class refractory. Thirteen (10%) subjects had prior selinexor therapy, and 21 (15%) had prior BCMA-directed therapy.

Most common all-grade hematologic AEs were anemia (52%), neutropenia (47%), and lymphopenia (41%). Most common all-grade nonhematologic AEs were CRS (47%), dysgeusia (33%), and fatigue (32%). Two dose-limiting toxicities of clinically asymptomatic grade 4 increased lipase in the setting of a pancreatic plasmacytoma (7.5 μg/kg IV; unresolved) and grade 3 maculopapular rash (135 μg/kg SC; resolved after 3 days) were reported. Treatment-related grade 3-4 AEs were reported in 50% of patients, with lymphopenia (21%) and neutropenia (16%) being most frequent. Infections were reported in 38% of patients, and treatment-related infusion/injection site reactions (IV and SC) were reported in 16%.

CRS was reported in 48% of patients, and all events were grade 1-2 except for 4 (3%) events of grade 3 severity. CRS was generally confined to the first cycle, and the severity appears to be mitigated by implementation of step-up dosing and SC administration. Neurotoxicity was reported in 7 (5%) patients; 4 had grade 1-2 events and 3 had grade 3 delirium (n=2) and confusion (n=1). Neurotoxicity events occurred in the context of CRS in 4 patients.

PK results from IV dosing indicated that the half-life of talquetamab supports weekly dosing. SC results showed lower Cm, with comparable trough levels than that of IV dosing (at a similar dose) which makes it a favorable administration option.

Talquetamab treatment led to pharmacodynamic changes supporting mechanism of action, including increases in T cell activation and cytokines such as IL-10, IL-2Ra and IL-6. Comparable induction of pharmacodynamic markers was seen with IV and SC dosing.

Overall response rates were 32% and 36% for IV and SC cohorts, respectively. In the more recent dose escalation cohorts, the response rates have continued to improve (IV: 20 μg/kg [67%], 60 μg/kg [100%]; SC: 135 μg/kg [50%], 405 μg/kg [100%]). Responses were noted early at the 1.0 μg/kg dose, and stringent complete responses were achieved starting at the 1.5 μg/kg dose.

TABLE 5
Summary of Best Overall Response

| | IV (Q2W and QW) | | | | | | |
|---|---|---|---|---|---|---|---|
| | 0.5- | | | | SC (QW) | | |
| Re- | 11.25 | 20 | 60 | 180 | 5-45 | 135 | 405 |
| sponse, | μg/kg | μg/kg | μg/kg | μg/kg | μg/kg | μg/kg | μg/kg |
| n (%) | (n = 83) | (n = 9) | (n = 6) | (n = 2) | (n = 13) | (n = 8) | (n = 3) |
| ORR | 21 (25) | 6 (67) | 6 (100) | 1 (50) | 2 (15) | 4 (50) | 3 (100) |
| ≥VGPR | 10 (12) | 5 (56) | 4 (67) | 0 | 0 | 1 (13) | 1 (33) |

Best Response

| | | | | | | | |
|---|---|---|---|---|---|---|---|
| sCR | 4 (5) | 0 | 1 (17) | 0 | 0 | 0 | 0 |
| CR | 1 (1) | 0 | 0 | 0 | 0 | 0 | 0 |
| VGPR | 5 (6) | 5 (56) | 3 (50) | 0 | 0 | 1 (13) | 1 (33) |
| PR | 11 (13) | 1 (11) | 2 (33) | 1 (50) | 2 (15) | 3 (38) | 2 (67) |
| MR | 2 (2) | 0 | 0 | 0 | 0 | 0 | 0 |
| SD | 39 (47) | 1 (11) | 0 | 1 (50) | 9 (69) | 3 (38) | 0 |
| PD | 16 (19) | 2 (22) | 0 | 0 | 2 (15) | 1 (13) | 0 |

CR: complete response;
IV: intravenous;
MR: minimal response;
QW, weekly;
Q2W: every 2 weeks;
ORR: overall response rate;
PD: progressive disease;
PR: partial response;
SC: subcutaneous;
sCR: stringent complete response;
SD: stable disease;
VGPR: very good partial response.

TABLE 6
Summary of Overall Best Confirmed Response based on Investigator Assessment;
Modified Intent-to-treat Analysis Set (Part 1, IV, Q2W)

| | IV Bi-weekly (g/kg) | | | | |
|---|---|---|---|---|---|
| | 0.5 | 1.0 | 1.5 | 2.25 | 3.38 |
| Analysis set: Modified intent-to | 1 | 2 | 10 | 10 | 2 |
| treat | | | | | |
| Response category | | | | | |
| Stringent complete response (sCR) | 0 | 0 | 2 (20.0%) | 0 | 0 |
| Complete response (CR) | 0 | 0 | 0 | 0 | 0 |
| Very good partial response (VGPR) | 0 | 1 (50.0%) | 1 (10.0%) | 1 (10.0%) | 0 |
| Partial response (PR) | 0 | 0 | 0 | 2 (20.0%) | 0 |
| Minimal response (MR) | 0 | 0 | 1 (10.0%) | 2 (20.0%) | 0 |
| Stable disease (SD) | 0 | 1 (50.0%) | 3 (30.0%) | 4 (40.0%) | 0 |
| Progressive disease (PD) | 1 (100.0%) | 0 | 3 (30.0%) | 1 (10.0%) | 2 (100.0%) |
| Not evaluable (NE) | 0 | 0 | 0 | 0 | 0 |
| Overall response | 0 | 1 (50.0%) | 3 (30.0%) | 3 (30.0%) | 0 |
| (sCR + CR + VGPR + PR) | | | | | |
| Clinical benefit (Overall response + | 0 | 1 (50.0%) | 4 (40.0%) | 5 (50.0%) | 0 |
| MR) | | | | | |
| VGPR or better (sCR + CR + | 0 | 1 (50.0%) | 3 (30.0%) | 1 (10.0%) | 0 |
| VGPR) | | | | | |

Note:
Response was assessed by investigators, based on International Uniform Response Criteria Consensus Recommendations. Percentages were calculated with the number of subjects in each group as denominator..

TABLE 7
Summary of Overall Best Confirmed Response based on Investigator Assessment; Modified Intent-to-treat
Analysis Set (Part 1, IV, QW)

| | IV Weekly (μg/kg) | | | | | | | | |
|---|---|---|---|---|---|---|---|---|---|
| | | | 1.5 | 1.5 | 1.5 | 1.5 | 1.5/1.5 | 1.5/10 | 1.5/10 |
| | 2.25 | 1.5 | then 3.38 | then 5 | then 7.5 | then 11.25 | then 20 | then 20 | then 60 |
| Analysis set: | 12 | 6 | 12 | 12 | 10 | 6 | 3 | 6 | 6 |
| Modified intent-to-treat | | | | | | | | | |
| Response category | 1 (8.3%) | 0 | 0 | 0 | 0 | 1 (16.7%) | 0 | 0 | 1 (16.7%) |
| Stringent complete response | | | | | | | | | |
| (sCR) | | | | | | | | | |
| Complete response (CR) | 1 (8.3%) | 0 | 0 | 0 | 0 | 0 | 0 | 0 | 0 |
| Very good partial response | 0 | 0 | 0 | 1 (8.3%) | 1 (10.0%) | 0 | 2 (66.7%) | 3 (50.0%) | 2 (33.3%) |
| (VGPR) | | | | | | | | | |
| Partial response (PR) | 2 (16.7%) | 0 | 2 (16.7%) | 1 (8.3%) | 2 (10.0%) | 2 (33.3%) | 0 | 1 (16.7%) | 2 (33.3%) |
| Minimal response (MR) | 0 | 0 | 0 | 0 | 0 | 0 | 0 | 0 | 0 |
| Stable disease (SD) | 6 (50.0%) | 6 (100.0%) | 6 (50.0%) | 6 (50.0%) | 5 (50.0%) | 2 (33.3%) | 0 | 1 (16.7%) | 1 (16.7%) |
| Progressive disease (PD) | 2 (16.7%) | 0 | 4 (33.3%) | 4 (33.3%) | 2 (20.0%) | 1 (16.7%) | 1 (33.3%) | 1 (16.7%) | 0 |
| Not evaluable (NE) | 0 | 0 | 0 | 0 | 0 | 0 | 0 | 0 | 0 |
| Overall response | 4 (33.3%) | 0 | 2 (16.7%) | 2 (16.7%) | 3 (30.0%) | 3 (50.0%) | 2 (66.7%) | 4 (66.7%) | 5 (83.3%) |
| (sCR + CR + VGPR + PR) | | | | | | | | | |
| Clinical benefit (Overall | 4 (33.3%) | 0 | 2 (16.7%) | 2 (16.7%) | 3 (30.0%) | 3 (50.0%) | 2 (66.7%) | 4 (66.7%) | 5 (83.3%) |
| response + MR) | | | | | | | | | |
| VGPR or better | 2 (16.7%) | 0 | 0 | 1 (8.3%) | 1 (10.0%) | 1 (16.7%) | 2 (66.7%) | 3 (50.0%) | 3 (50.0%) |
| (sCR + CR + VGPR) | | | | | | | | | |

Note:
Response was assessed by investigators, based on International Uniform Response Criteria Consensus Recommendations. Percentages are calculated with the number of subjects in each group as denominator.

TABLE 8
Summary of Overall Best Confirmed Response based on Investigator Assessment;
Modified Intent-to-treat Analysis Set (Part 1, SC, QW)

| | IV Weekly | SC | | | | | | | | |
|---|---|---|---|---|---|---|---|---|---|---|
| | (μg/kg) | | Weekly (μg/kg) | | | | | | | |
| | 1.5/10/60 | IV | 1.5 | 5 | 10 | 10/45 | 10/60 | 10/60/300 | | |
| | then 180 | Total | then 5 | then 15 | then 45 | then 135 | then 405 | then 800 | Total | Total |
| Analysis set: | 2 | 100 | 4 | 3 | 6 | 8 | 3 | 1 | 25 | 125 |
| Modified intent-to-treat | | | | | | | | | | |
| Response category | 0 | 5 (5.5%) | 0 | 0 | 0 | 0 | 0 | 0 | 0 | 5 (4.0%) |
| Stringent complete response | | | | | | | | | | |
| (sCR) | | | | | | | | | | |
| Complete response (CR) | 0 | 1 (1.0%) | 0 | 0 | 0 | 0 | 0 | 0 | 0 | (1 (0.8%) |
| Very good partial response | | 12 | | | | | | | | 14 |
| (VGPR) | 0 | (12.0%) | 0 | 0 | 0 | 1 (12.5%) | 1 (33.3%) | 0 | 2 (8.0%) | (11.2%) |
| Partial response (PR) | | 14 | | | | | | | | 21 |
| | | (14.0%) | 1 (25.0%) | 0 | 1 (16.7%) | 3 (37.5%) | 2 (66.7%) | 0 | 7 (28.0%) | (16.8%) |
| Minimal response (MR) | 0 | 3 (3.0%) | 0 | 0 | 0 | 0 | 0 | 0 | 0 | 3 (2.4%) |
| Stable disease (SD) | 2 | 43 | | 3 | | | | 1 | 13 | 56 |
| | (100.0%) | (43.0%) | 3 (75.0%) | (100.0%) | 3 (50.0%) | 3 (37.5%) | 0 | (100.0%) | (52.0%) | (44.8%) |
| Progressive disease (PD) | | 22 | | | | | | | | 25 |
| | 0 | (22.0%) | 0 | 0 | 2 (33.3%) | 1 (12.5%) | 0 | 0 | 3 (12.0%) | (20.0%) |
| Not evaluable (NE) | 0 | 0 | 0 | 0 | 0 | 0 | 0 | 0 | 0 | 0 |
| Overall response | | 32 | | | | | 3 | | | 41 |
| (sCR + CR + VGPR + PR) | 0 | (32.0%) | 1 (25.0%) | 0 | 1 (16.7%) | 4 (50.0%) | (100.0%) | 0 | 9 (36.0%) | (32.8%) |
| Clinical benefit (Overall | | 35 | | | | | 3 | | | 44 |
| response + MR) | 0 | (35.0%) | 1 (25.0%) | 0 | 1 (16.7%) | 4 (50.0%) | (100.0%) | 0 | 9 (36.0%) | (35.2%) |
| VGPR or better | | 18 | | | | | | | | 20 |
| (sCR + CR + VGPR) | 0 | (18.0%) | 0 | 0 | 0 | 1 (12.5%) | 1 (33.3%) | 0 | 2 (8.0%) | (16.0%) |

Note:
Response was assessed by investigators, based on International Uniform Response Criteria Consensus Recommendations. Percentages are calculated with the number of subjects in each group as denominator.

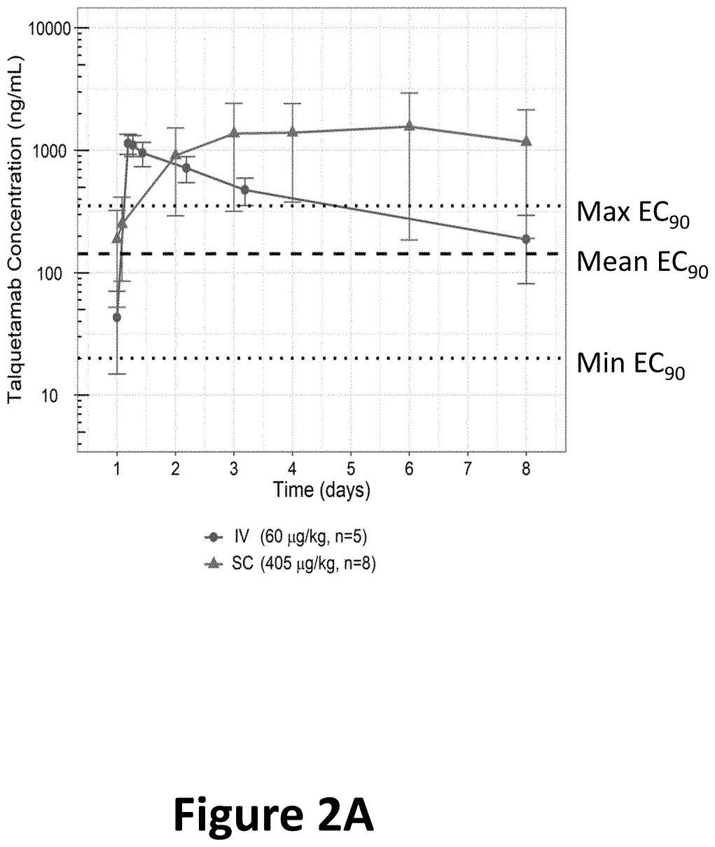

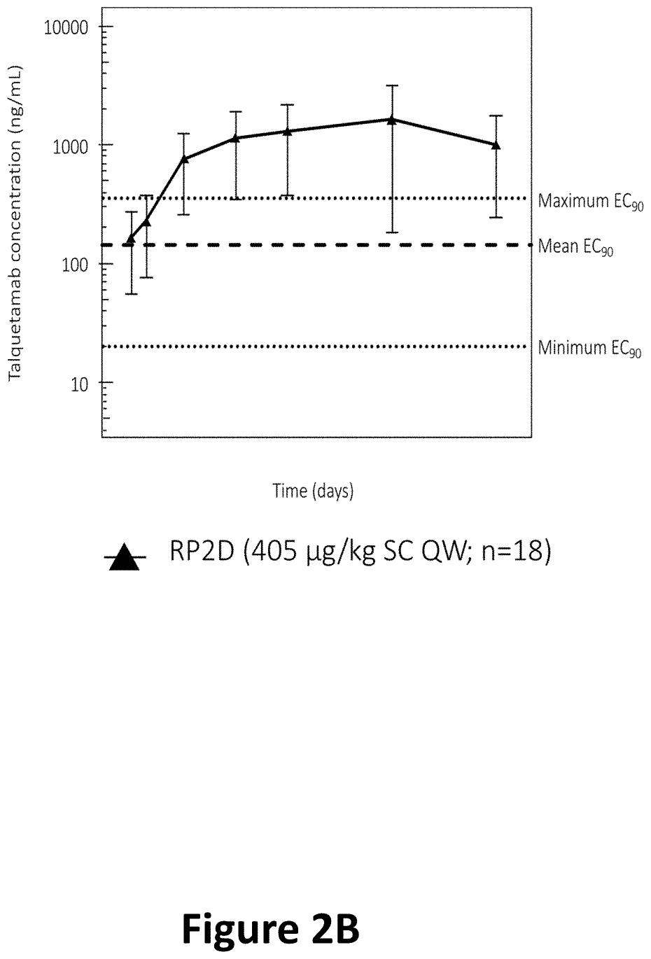

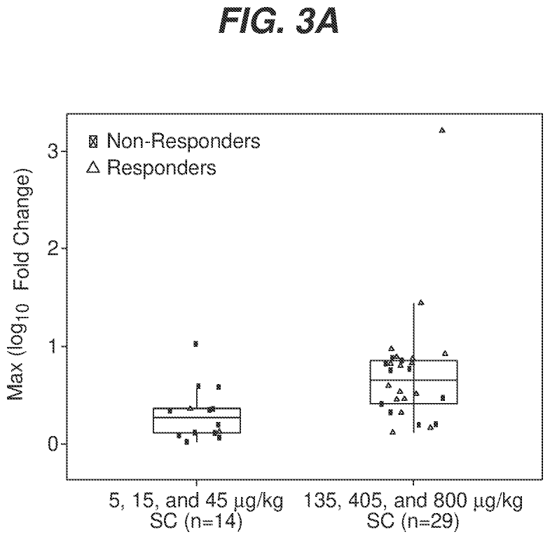

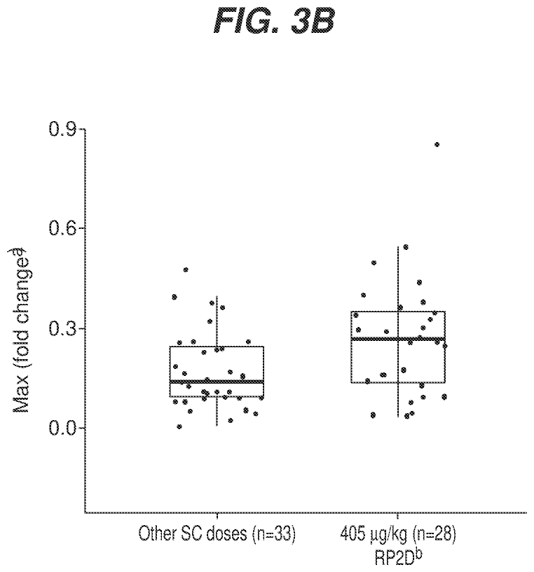

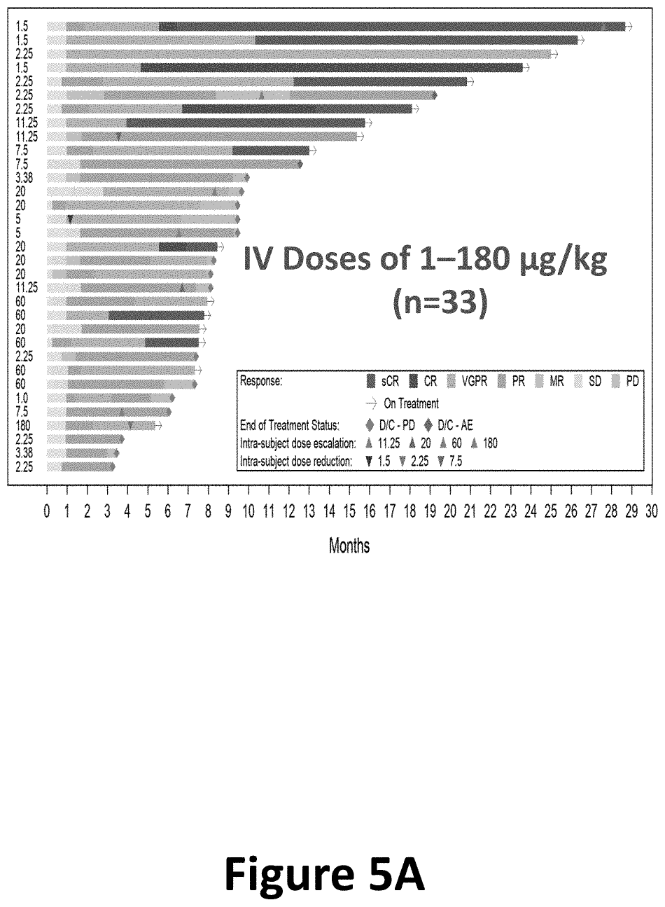

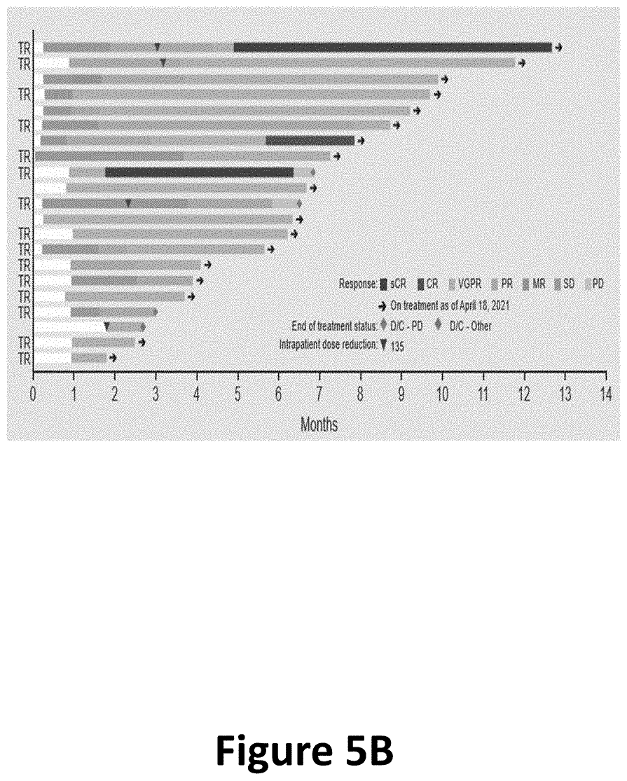

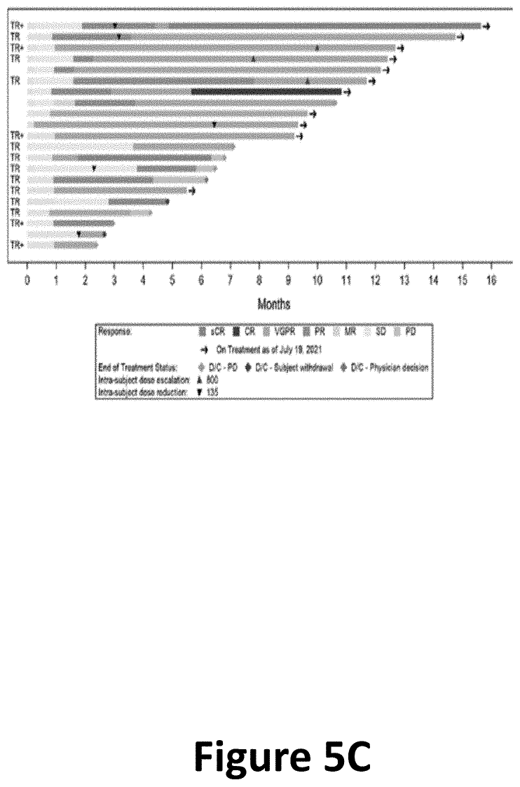

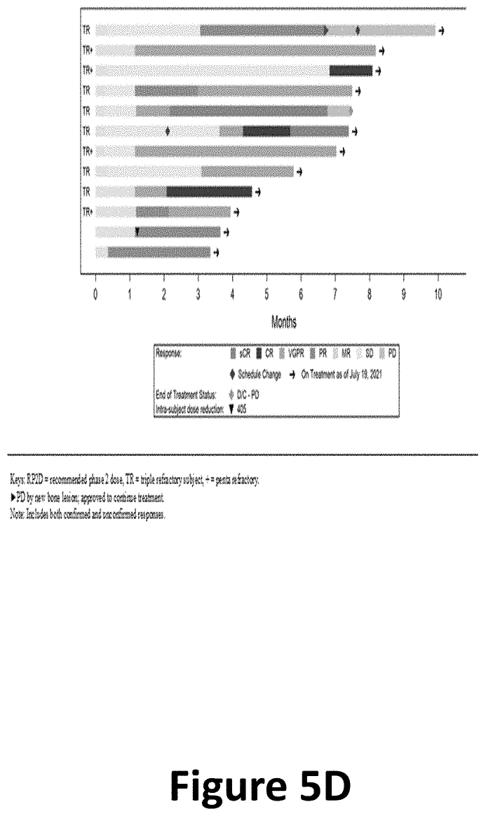

Further data analysis provided pharmacokinetics data support for RP2D (e.g., FIGS. 2A, 3A, and 3B), overall response rate for SC doses, and duration responses (e.g., FIGS. 5A-5D). The mean PK profile following first treatment dose (FIG. 2A) shows that the exposure was dose-proportional following first dose in 5-405 μg/kg SC cohorts. 405 μg/kg SC cohorts had lower peak/trough ratio than 60 μg/kg IV cohorts and maintained exposure over the maximum EC₉₀. Thus, there is opportunity for less frequent SC dosing. In addition, ADA (anti-drug antibody) rate in patients was 12% (11/95) for IV and 8% (3/38) for SC. ADA did not appear to impact safety, PK, or efficacy. And as demonstrated by FIGS. 3A and 3B, consistent induction of cytokines (IL-10, IL-6, IL2Ra) was observed at doses >45 μg/kg SC; PD-1⁺ T cells were induced in the periphery, indicative of T cell activation; and consistent T cell activation was observed at RP2D of 405 μg/kg SC.

The data analysis also demonstrates that, 1) at most active doses of 20-180 μg/kg IV and 235-800 μg/kg SC, the ORR was 66% (33/50), ≥VGPR was 42%, and the responses deepened over time; 2) at the RP2D of 405 μg/kg SC, the ORR was 69% (9/13), the median time to first confirmed response was 1 month (1-2), 6/9 (67%) of the responders were triple-class refractory, and 2/9 (22%) of the responders were penta-drug refractory. Additionally, duration of response (DPR) data (FIG. 5A, the data for IV cohorts were more mature at time of analysis) shows that the responses were durable and deepened over time and the median time to first confirmed response across all doses was 1 month (0.2-3). In responders with duration of >12 months, 9/10 were still in response, with 6≥CR and 4 with DOR of >2 years. 1 of 6 responders at doses ≥60 μg/kg IV, had progressed at median 7.4-month (5.1-7.8) follow-up. None of the 17 responders at doses ≥405 μg/kg SC, had progressed at median 3.7-month (1.4-6.5) follow-up.

### Part 1 (Dose Escalation Part): Additional Patients and Longer Follow-Up for the SC Administration (Cut-Off Date for Analyses Apr. 18, 2021)

Subcutaneous (SC) administration of escalating doses of talquetamab (5-800 μg/kg) with and without step-up dosing were assessed in additional cohorts and longer follow-up time using the same study design described above (FIG. 1i). Patients must have had measurable disease and have progressed on or could not tolerate all available established therapies. Prior BCMA-targeted therapy was allowed. Premedications (i.e., glucocorticoid, antihistamine, and antipyretic) were limited to step-up doses and the first full dose; however, there was no steroid requirement after the first full dose.

Among the 82 in total SC subjects evaluated, 30 received talquetamab by SC, once weekly (QW), at the RP2D of Q405 g/kg with step-up doses of 10 and 60 μg/kg. The other patients received SC, QW or biweekly doses of 5, 15, 45, 135, or 800 μg/kg talquetamab. Median age range for the 82 subjects evaluated was 63 years (range 42-80; 270 were 70), median time since diagnosis was 5.9 years (range 1-20 years), and 1600 had International Staging System stage III disease. Median number of prior therapies was 6 (range 2-17), 840 of subjects were refractory to last line of therapy, 76 triple-class refractory, 78 penta-class exposed, and 280 penta-class refractory. Twenty (240%) of subjects had prior BCMA-directed therapy. A summary of the subjects' demographics and disease characteristics is shown in Table 9.

TABLE 9
Summary of Patient Demographics and Disease
Characteristics (SC, QW)

| | | RP2D |
|---|---|---|
| | SC Total | (405 μg/kg SC QW)^{a} |
| Characteristic | n = 82 | n = 30 |
| Median age (range), y | 63.0 (42-80) | 61.5 (46-80) |
| Aged ≥70 y, n (%) | 22 (27) | 7(23) |
| Sex, n (%) | | |
| Male | 47 (57) | 19 (63) |
| Female | 35(43) | 11(37) |
| Median time since diagnosis | 5.9 (1-20) | 5.6 (2-20) |
| (range), y | | |
| Extramedullary plasmacytomas | 27 (33) | 10 (33) |
| ≥1. N (%)^{b} | | |
| Bone marrow plasma cells ≥ | 13(17) | 6(21) |
| 60%, n(%)^{c} | | |
| ISS stage, n (%)^{d} | | |
| I | 26 (32) | 12 (40) |
| II | 36 (44) | 13 (43) |
| III | 13 (16) | 3 (10) |
| Prior transplantation, n (%) | 71 (87) | 27 (90) |
| Median no. prior lines of therapy | 6.0 (2-17) | 6.0 (2-14) |
| (range) | | |
| Exposure status, n (%) | | |
| Prior BCMA therapy^{e} | 20 (24) | 8 (27) |
| Triple-class^{f} | 81 (99) | 30 (100) |
| Penta-drug^{g} | 64 (78) | 24 (80) |
| Refractory Status, n (%) | | |
| Pi^{h} | 69 (84) | 25 (83) |
| Carfilzomib | 54 (66) | 19 (63) |
| IMiDⁱ | 76 (93) | 28 (93) |
| Pomalidomide | 67 (82) | 26 (87) |
| Anti-CD38 mAb^{j} | 77 (94) | 30 (100) |
| BCMA^{e} | 14 (17) | 5 (16) |
| Triple-class^{f} | 62 (76) | 23 (77) |
| Penta-drugs^{g} | 23 (28) | 6 (20) |
| To last line of therapy | 69 (84) | 26 (87) |

BCMA = B-cell maturation antigen;
CAR-T = chimeric antigen receptor T-cell;
IMiD = immunomodulatory drug;
ISS = International Staging System;
mAb = monoclonal antibody;
PI = proteasome inhibitor;
QW = weekly;
RP2D = recommended phase 2 dose;
SC = subcutaneous
^{a}Step-up doses of 10 and 60 ug/kg.
^{b}Soft-tissue component of a bone-based plasmacytoma not included.
^{c}Percentages calculated from n = 76 for SC total and n = 29 at RP2D.
^{d}Percentages calculated from n = 66 SC for total and n = 27 at RP2D.
^{e}BCMA CAR-T therapy or BCMA non-CAR-T therapy.
^{f}≥1 PI, ≥1 IMiD, and 1 anti-CD38 mAb.
^{g}≥2 PI, ≥2 IMiD, and 1 anti-CD38 mAb.
^{h}Bortezomib, carfilzomib, and/or ixazomib.
^{j}Thalidomide, lenalidomide, and/or pomalidomide.
^{j}Daratumumab and/or isatuximab.

The study showed that talquetamab has a tolerable safety profile at the RP2D of 405 μg/kg. No dose-limiting toxicities and deaths due to AEs were observed at the RP2D. Cytopenias were mostly confined to the step-dosing and to the first and second treatment cycles. Neutropenias generally resolved within a week and were limited to the first and second treatment cycles. Infections were observed in 37% of the patients evaluated (9% for grade 3 or 4) and in 32% of patients at the RP2D (3% for grade 3 or 4). Neurotoxicities were observed in 4 patients with SC dosing (all grade 1 or 2) and in 2 patients (7%) at the RP2D. Injection-site reactions occurred in 17% of patients, including at the RP2D), but were mild and manageable (all grade 1 or 2). Skin-related AEs (includes skin exfoliation, pruritis, rash, and nail disorders) occurred in 67% of patients and in 77% of patients at the RP2D (majority grade 1 or 2). Nail disorders (includes onychomadesis and nail dystrophy) occurred in 21% of patients (27% of patients at the RP2D).

Most common all-grade hematologic AEs were neutropenia (57% at any grade, 49% at grade 3 or 4), anemia (45% at any grade, 28% at grade 3 or 4), leukopenia (26% at any grade, 20% at grade 3 or 4), and thrombocytopenia (28% at any grade, 18% at grade 3 or 4). Most common all-grade nonhematologic AEs were CRS (67% at any grade, 1% at grade 3 or 4), dysgeusia (46% at any grade, not applicable at grade 3 or 4), and fatigue (32% at any grade, 0% at grade 3 or 4). Most common all-grade nonhematologic AEs observed at the RP2D of 405 μg/kg were CRS (73% at any grade, 2% at grade 3 or 4), dysgeusia (60% at any grade, NA at grade 3 or 4), and dysphagia (3700 at any grade, 000 at grade 3 or 4). A summary of the data for the safety profile of talquetamab is shown in Table 10.

TABLE 10
Safety Profile of Talquetamab (Part 2, SC, QW)

| | SC Total | | RP2D (405 μg/kg SC QW)^{a} | |
|---|---|---|---|---|
| AE(≥20% of | n = 82 | | n = 30 | |
| Total SC), n (%) | Any Grade | Grade 3/4 | Any Grade | Grade 3/4 |

Hematologic

| | | | | |
|---|---|---|---|---|
| Anemia | 37 (45) | 23 (28) | 17 (57) | 8 (27) |
| Neutropenia | 47 (57) | 40 (49) | 20 (67) | 18 (60) |
| Lymphopenia | 19 (23) | 19 (23) | 9 (30) | 9 (30) |
| Thrombocytopenia | 23 (28) | 15 (18) | 10 (33) | 6 (20) |
| Leukopenia | 21(26) | 16 (20) | 11 (37) | 8 (27) |

Nonhematologic

| | | | | |
|---|---|---|---|---|
| CRS | 55 (67) | 1(1) | 22 (73) | 1(2) |
| Dysgeusia | 38 (46) | NA | 18 (60) | NA |
| Fatigue | 26 (32) | 0 | 9 (30) | 0 |
| Headache | 19 (23) | 1(1) | 7 (23) | 0 |
| Pyrexia | 23 (28) | 1 (1) | 7 (23) | 1 (2) |
| Dry mouth | 22 (27) | 0 | 8 (27) | 0 |
| Dysphagia | 21 (26) | 0 | 11(37) | 0 |
| Diarrhea | 18 (22) | 0 | 7 (23) | 0 |
| Nausea | 12 (22) | 0 | 7 (23) | 0 |

AEs = adverse events,
CRS = cytokine release syndrome;
DLT = dose-limiting toxicity;
NA = not applicable;
RP2D = recommended phase 2 dose;
SC = subcutaneous
^{a}Step-up doses of 10 and 60 μg/kg.
^{b}includes skin exfoliation, pruritis, rash, and nail disorders
^{c}includes nail disorders, onychomadesis and nail dystrophy.

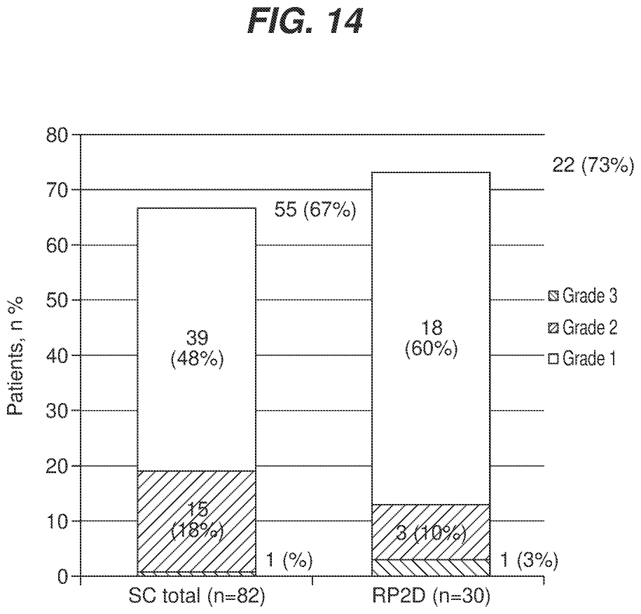

CRS was generally limited to grade 1 or 2 in all subjects (with the exception of one patient with grade 3 CRS) and the severity appears to be mitigated by implementation of step-up dosing and SC administration (FIG. 14). Median time to CRS onset was 2 days (range 1-22 days) and median duration of CRS was 2 days (range 1-7 days). Out of the 82 patients treated with SC talquetamab, 67 received supportive measures to treat their CRS (e.g., tocilizumab, steroids, low-flow oxygen by nasal cannula, and vasopressor). The majority of patients only had 1 dose of tocilizumab as a supportive measure for CRS. A summary of the data pertaining to patients that experienced CRS is shown in Table 11.

TABLE 11
Cytokine Release Syndrome that Occurred Following Treatment with
Talquetamab (Part 2, SC, QW)

| | SC total | RP2D (405 μg/kg SC QW)^{a} |
|---|---|---|
| Parameter | n = 82 | n = 30 |
| Patients with CRS, n (%) | 55 (67) | 22 (73) |
| Median time to onset (range), days^{b} | 2 (1-22) | 2 (1-22) |
| Median duration (range), days | 2 (1-7) | 2 (1-3) |
| Supportive measures, n (%)^{c} | 55 (67) | 22 (73) |
| Tocilizumab^{d} | 43 (52) | 18 (60) |
| Steroids | 5 (6) | 1 (3) |
| Low-flow oxygen by nasal | 6 (7) | 1 (3) |
| cannula | | |
| Vasopressor | 2 (2) | 1 (3) |

CRS = cytokine release syndrome;
QW = weekly;
RP2D = recommended phase 2 dose;
SC = subcutaneous
^{a}Step-up doses of 10 and 60 μg/kg.
^{b}Relative to the most recent dose.
^{c}A patient could receive >1 supportive therapy.
^{d}Tocilzumab was allowed for grade 1 CRS
^{e}Graded according to Lee et al.Blood. 2014.124:188.

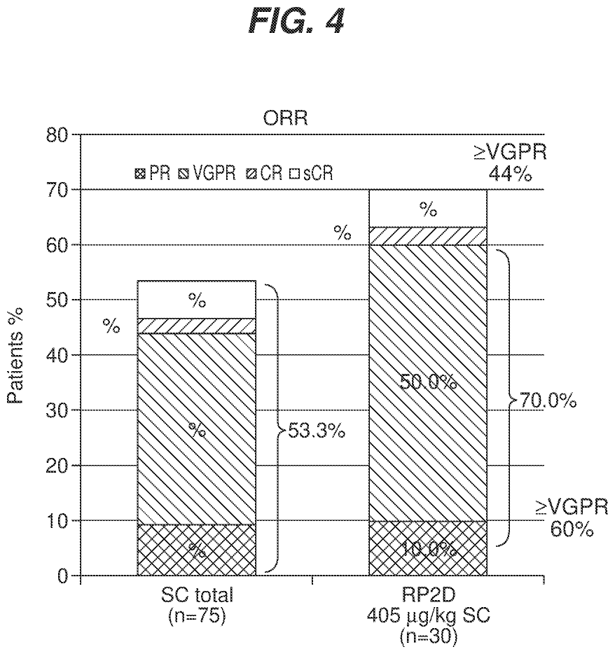

The RP2D of 405 μg/kg SC QW was administered to 30 patients with a median follow-up of 6.3 months (range 1.4-12 months) for the responders. The data analysis (FIG. 4) demonstrates that for the escalating doses of talquetamab (5-800 μg/kg) in 75 patients, note that not all 82 patients were available for evaluation) the ORR was 53.3% (40/75) and ≥VGPR was 44%; 2) at the RP2D of 405 μg/kg SC, the ORR was 70% (21/30), ≥VGPR was 60%, the median time to first confirmed response was 1 month (range 0.2-3.8 months), 15/23 (65.2%) of the responders were triple-class refractory, and 5/6 (83.3%) of the responders were penta-drug refractory. The ORR was assessed in evaluable patients who had ≥1 dose of talquetamab and ≥1 post-baseline disease evaluation per the 2011 International Myeloma Working Group response criteria. Out of 6 evaluable patients across the IV and SC cohorts, 4 had negative MRD CR/sCR at 10⁻⁶, including 1 subject in the RP2D cohort. Negative MRD was sustained 7 months post-complete response in 1 evaluable patient.

Additionally, duration of response data (FIG. 5B) show that the responses were durable and deepened over time in 40 patients treated with SC doses of talquetamab ranging from 45 to 800 μg/kg. At the RP2D of 405 μg/kg SC QW (FIG. 5C), median duration of response was not reached and after median follow-up of 6.3 months (range 1.4-12.2+ months), 17/21 responders (81%) were alive and remained on talquetamab treatment. Across all SC cohorts, after median follow-up of 6.8 months (range 1.4-16.3+ months) 31/40 responders (78%) remained on talquetamab treatment. The data for IV cohorts (FIG. 5A) was more mature and even at subtherapeutic doses, responses were ongoing at 22+ months in patients with longer follow-up.

Further data analysis provided pharmacokinetics (PK) data support for the SC RP2D of 405 μg/kg (FIG. 2B). The RP2D exhibited a low peak/trough ratio and maintained exposure over the maximum EC₉₀. In 6 out of 50 patients (12%) treated with SC talquetamab, the patients exhibited anti-drug antibodies that generally were of low titer. Additionally, the anti-drug antibodies did not appear to impact safety, PK, or efficacy. Post-talquetamab administration, PD-1 positive T cells were induced in the periphery, which indicated T cell activation (FIG. 3B). Consistent induction of PD-1+ T cells was observed in the RP2D cohorts. Moreover, consistent induction of cytokines (i.e., IL-10, IL-6, IL-2Rα) was observed at dosses greater than 45 μg/kg SC.

In addition to 405 μg/kg, 800 μg/kg of talquetamab was also well tolerated and highly effective. Patients were treated weekly or biweekly with 800 μg/kg of talquetamab.

### Conclusion, First Data Cutoff

Talquetamab had a manageable safety profile across all doses assessed: most CRS events (67%) were grade 1-2 and generally confined to first step-up and full doses; step-up dosing mitigated high-grade CRS; there was a low incidence of neurotoxic events which were predominantly grade 1-2. Talquetamab specific skin-related AEs (includes skin exfoliation, pruritis, rash, and nail disorders) occurred in 67% of patients.

Greater responses were reached at higher doses: the data analysis demonstrates that for the escalating doses of talquetamab (5-800 μg/kg) in 75 patients, note that not all 82 patients were available for evaluation the ORR was 53.3% (40/75) and >VGPR was 44%; 2) at the RP2D of 405 μg/kg SC, the ORR was 70%. Additionally, duration of response data shows that the responses were durable and deepened over time.

### Part 1 and 2: Additional Patients and Longer Follow-Up for the SC Administration (Cut-Off Date for Analyses Jul. 19, 2021)

As of 19 Jul. 2021, 97 subjects have been enrolled in Parts 1 and 2 for talquetamab SC dosing and received at least 1 dose of talquetamab. Thirty subjects (including Part 2 subjects) have been enrolled to receive 10 and 60 μg/kg step-up doses followed by a 405 μg/kg SC weekly treatment dose (the first selected RP2D). Twenty-three subjects (including Part 2 subjects) have been enrolled to receive 10, 60 and 300 μg/kg step-up doses followed by a 800 μg/kg SC biweekly treatment dose (the second selected RP2D).

The median age for the 30 subjects receiving talquetamab SC at the RP2D of 405 μg/kg SC weekly was 61.5 years (range: 46 to 80 years), with 7 (23.3%) subjects ≥70 years of age. The median number of prior therapeutic regimens was 6 (range: 2 to 14). All 30 subjects (100%) were prior triple-exposed (prior therapy included PI, IMiD, and anti-CD38 monoclonal antibody) and 80.0% were prior penta-exposed (prior therapy included 2 or more PIs, 2 or more IMiDs, and an anti-CD38 monoclonal antibody). Notably, all 30 subjects were refractory to anti-CD38 monoclonal antibody therapy, 76.7% were triple-refractory, and 20.0% were penta-refractory.

The median age for the 23 subjects receiving talquetamab SC at the RP2D of 800 μg/kg SC biweekly was 60.0 years (range: 47 to 84 years), with 7 (30.4%) subjects ≥70 years of age. The median number of prior therapeutic regimens was 5 (range: 1 to 17). Twenty-two subjects (95.7%) were prior triple-exposed (prior therapy included PI, IMiD, and anti-CD38 monoclonal antibody) and 69.6% were prior penta-exposed (prior therapy included 2 or more PIs, 2 or more IMiDs, and an anti-CD38 monoclonal antibody). Notably, 78.3% were refractory to anti-CD38 monoclonal antibody therapy, 65.2% were triple-refractory, and 21.7% were penta-refractory.

The median age for the 97 subjects receiving talquetamab SC at any dosage was 64.0 years (range: 39 to 84 years), with 28 (28.9%) subjects ≥70 years of age. The median number of prior therapeutic regimens was 6 (range: 1 to 17). Ninety-six subjects (99.0%) were prior triple-exposed (prior therapy included PI, IMiD, and anti-CD38 monoclonal antibody) and 78.4% were prior penta-exposed (prior therapy included 2 or more PIs, 2 or more IMiDs, and an anti-CD38 monoclonal antibody). Notably, 90.7% of subjects were refractory to anti-CD38 monoclonal antibody therapy, 71.1% were triple-refractory, and 22.7% were penta-refractory.

Another 102 subjects have been treated with talquetamab IV doses in this study. The efficacy and safety profiles of talquetamab IV are comparable to those of talquetamab SC.

### Efficacy

Talquetamab RP2D of 405 μg/Kg SC Weekly

Disease assessment was done by investigator based on 2011 IMWG response criteria. As of the data cutoff, all 30 subjects treated with talquetamab at the RP2D of 405 μg/kg SC weekly had ≥1 postdose disease evaluation (i.e., response evaluable population). The median follow-up as of the data cutoff for the 30 subjects was 11.27 months (range of 4.2 to 15.2 months), and the ORR was 70.0% (Table 12). Among them, 2 subjects (6.7%) had sCR, 1 subject (3.3%) had CR, 14 subjects (46.7%) had VGPR, and 4 subjects (13.3%) had PR as the best confirmed response. No subject had progressive disease as the best confirmed response, and 9 subjects (30.0%) had stable disease (Table 12). Among responders, the median time to first confirmed response (PR or better) was 0.92 months (range: 0.2 to 5.4 months), and the median duration of response as of the data cutoff has not been reached.

Talquetamab RP2D of 800 μg/Kg SC Biweekly

Among the 23 subjects receiving talquetamab at the RP2D of 800 μg/kg SC biweekly, 18 subjects were response evaluable by investigators. The median follow-up as of the data cutoff for the 30 subjects was 3.65 months (range of 0.0 to 12.0 months), and the ORR was 66.7% (Table 12). Among them, 2 subjects (11.1%) had sCR, 2 subjects (11.1%) had CR, 5 subjects (27.8%) had VGPR, and 3 subjects (16.7%) had PR as the best confirmed response. No subject had progressive disease as the best confirmed response, and 6 subjects (33.3%) had stable disease (Table 12). Among responders, the median time to first confirmed response (PR or better) was 1.17 months (range: 0.4 to 11.1 months), and the median duration of response was 5.62 months (95% CI 3.71, NE).

### All Talquetamab SC Cohorts

Among the 97 subjects receiving any dose of talquetamab SC (with a median follow-up of 7.46 months [range of 0.0 to 18.0 months]), 84 were response evaluable by investigators. The ORR was 56.0% for these 84 subjects. Among them, 6 subjects (7.1%) had sCR, 4 subjects (4.8%) had CR, 26 subjects (31.0%) had VGPR, and 11 subjects (13.1%) had PR as the best confirmed response (Table 12). Among responders, the median time to first confirmed response (PR or better) was 1.15 months (range: 0.2 to 11.1 months), and the median duration of response as of the data cutoff has not been reached.

TABLE 12
Summary of Overall Best Confirmed Response Based on Investigator
Assessment; Response Evaluable Responder Subjects by Investigators
(data cutoff 19 July 2021)

| | | SC Biweekly | |
|---|---|---|---|
| | SC Weekly 10/60 | 10/60/300 then | |
| | then 405* (SC20 | 800* (SC24, SC26 | |
| | and SC23) (μg/kg) | and SC27) (μg/kg) | SC Total |
| Analysis set: Response evaluable by | 30 | 18 | 84 |
| investigator for subjects | | | |
| Response category | | | |
| Stringent complete response (sCR) | 2 (6.7%) | 2 (11.1) | 6 (6.1%) |
| Unconfirmed | 0 | 1 | 1 |
| Complete response (CR) | 1(3.3%) | 2 (11.1) | 4 (4.8%) |
| Very good partial response (VGPR) | 14 (46.7%) | 5 (27.8) | 26 (31.0%) |
| Partial response (PR) | 4 (13.3%) | 3 (16.7) | 11(13.1%) |
| Unconfirmed | 0 | 0 | 1 |
| Minimal response (MR) | 0 | 0 | 0 |
| Stable disease (SD) | 9 (30.0%) | 6 (33.3) | 34 (40.5%) |
| Progressive disease (PD) | 0 | 0 | 3 (3.6%) |
| Not evaluable (NE) | 0 | 0 | 0 |
| Overall response | 21(70.0%) | 12 (66.7%) | 47 (56.0%) |
| (sCR + CR + VGPR + PR) | | | |
| Clinical benefit (Overall response + MR) | 21(70.0%) | 12 (66.7%) | 47 (56.0%) |
| CR or better (sCR + CR) | 3 (10.0%) | 4 (22.2%) | 10 (11.9%) |
| VGPR or better (sCR + CR + VGPR) | 17 (56.7%) | 9 (50.0%) | 36 (42.9%) |

Note:
Response evaluable subjects by investigators: Subjects have received at least one study treatment and have at least one post-baseline response evaluation by investigator. Response was assessed by investigators, based on IMWG Criteria. Percentages are calculated with the number of subjects in each group as denominator.
*Includes Part 2 subjects.

### Safety

### Treatment-Emergent Adverse Events

Talquetamab was evaluated in both IV and SC administration routes, which exhibited similar safety profiles. SC administration provides a more convenient treatment option for patients and healthcare workers, only the SC administration is being developed moving forward. Therefore, data presented below summarize the experience with talquetamab SC administration.

Talquetamab RP2D of 405 μg/Kg SC Weekly

All 30 subjects receiving talquetamab at the RP2D of 405 μg/kg SC Weekly as of the data cutoff date had at least 1 TEAE (Table 13).

The most frequently reported TEAEs for subjects treated with talquetamab at the RP2D of 405 μg/kg SC Weekly (≥20% of subjects) were cytokine release syndrome (CRS) (76.7%); neutropenia (66.7%); anemia, dysgeusia (60.0% each); lymphopenia, leukopenia (40.0% each); thrombocytopenia, dysphagia, skin exfoliation (36.7% each); fatigue, nail disorder (30.0% each); dry mouth, hypophosphatemia, pruritus (26.7% each); headache, diarrhea, nausea, rash, weight decreased (23.3% each); pyrexia, dry skin, alanine aminotransferase increased, gamma-glutamyltransferase increased, oropharyngeal pain (20.0% each).

No subject receiving the RP2D of 405 μg/kg SC Weekly had a TEAE that led to treatment discontinuation (Table 13).

Talquetamab RP2D of 800 μg/Kg SC Biweekly

Among the 23 subjects receiving talquetamab at the RP2D of 800 μg/kg SC biweekly as of the data cutoff date, 91.3% had at least 1 TEAE (Table 13).

The most frequently reported TEAEs for subjects treated with talquetamab at the RP2D of 800 μg/kg SC biweekly (≥20% of subjects) were CRS (78.3%); neutropenia, dry mouth (43.5% each); dysgeusia, fatigue, skin exfoliation, aspartate aminotransferase increased (30.4% each); anemia, dry skin, alanine aminotransferase increased (26.1% each); and lymphopenia, thrombocytopenia, decreased appetite, hypokalaemia (21.7% each).

No subject receiving the RP2D of 800 μg/kg SC biweekly had a TEAE that led to treatment discontinuation (Table 13).

### All Talquetamab SC Cohorts

Among the 97 subjects receiving any dose of talquetamab SC as of the data cutoff, 95.9% had at least 1 TEAE (Table 13).

The most frequently reported TEAEs (≥20% of subjects) were CRS (70.1%); neutropenia (54.6%); anemia (46.4%); dysgeusia (45.4%); thrombocytopenia, skin exfoliation (30.9% each); lymphopenia, leukopenia, fatigue (28.9% each); dry mouth (25.8%); pyrexia (23.7%); dysphagia, alanine aminotransferase increased (22.7% each); nausea, nail disorder (21.6% each); diarrhea, weight decreased (20.6% each).

Two subjects (2.1%) had a TEAE that led to treatment discontinuation (Table 13): 1 subject experienced Grade 3 cardiac failure related to prior chemotherapy regimens (not related to study drug), and 1 subject experienced Grade 1 maculopapular rash (related to study drug; the worst grade was Grade 3; the subject withdrew consent while the event improved to Grade 1).

TABLE 13
Overall Summary of Treatment-emergent Adverse Events; Safety
Analysis Set (data cutoff 19 July 2021)

| | SC Weekly | SC Biweekly | |
|---|---|---|---|
| | (μg/kg) | (μg/kg) | |
| | 10/60 then 405* | 10/60/300 then 800* | SC Total |
| Analysis set: Safety | 30 | 23 | 97 |
| Any TEAE | 30 (100.0%) | 21(91.3%) | 93 (95.9%) |
| Drug-related | 30 (100.0%) | 21(91.3%) | 89 (91.8%) |
| Number of events | 516 | 236 | 1236 |
| Relatedness | | | |
| Possible | 284 | 104 | 652 |
| Probable | 69 | 27 | 177 |
| Very likely | 163 | 105 | 407 |
| Doubtful | 28 | 32 | 93 |
| Not related | 177 | 72 | 667 |
| Unassigned | 0 | 0 | 10 |
| Maximum severity | | | |
| of any TEAE | | | |
| Grade 1 | 0 | 1(4.3%) | 3 (3.1%) |
| Grade 2 | 5 (16.7%) | 3 (13.0%) | 11(11.3%) |
| Grade 3 | 8 (26.7%) | 13 (56.5%) | 42 (43.3%) |
| Grade 4 | 17 (56.7%) | 3 (13.0%) | 35 (36.1%) |
| Grade 5 | 0 | 1(4.3%) | 2(2.1%) |
| Any serious TEAE | 10 (33.3%) | 5 (21.7%) | 37 (38.1%) |
| Number of events | 12 | 5 | 61 |
| Drug-related | 5 (16.7%) | 1(4.3%) | 13 (13.4%) |
| Number of events | 6 | 1 | 17 |
| Treatment | 0 | 0 | 2(2.1%) |
| discontinuation | | | |
| due to TEAE^{a} | | | |
| Drug-related | 0 | 0 | 1(1.0%) |
| Any does-limiting | 0 | 1(4.3%) | 3 (3.1%) |
| toxicity | | | |
| TEAE | | | |
| Number of events | 0 | 1 | 3 |
| Any CRS | 23 (76.7%) | 18 (78.3%) | 68 (70.1%) |
| Number of events^{b} | 34 | 25 | 93 |
| Serious events^{b} | 2 | 0 | 6 |
| Maximum severity | | | |
| of any CRS | | | |
| Grade 1 | 18 (60.0%) | 12 (52.2%) | 49 (50.5%) |
| Grade 2 | 4(13.3%) | 6 (26.1%) | 18(18.6%) |
| Grade 3 | 1(3.3%) | 0 | 1(1.0%) |
| Grade 4 | 0 | 0 | 0 |
| Grade 5 | 0 | 0 | 0 |
| Drug-related | 23 (76.7%) | 18 (78.3%) | 68 (70.1%) |
| Death due to TEAE^{c} | 0 | 1 (4.3%) | 2 (2.1%) |
| Drug-related | 0 | 0 | 0 |
| Potential Neurotoxicity | 5 (16.7%) | 1 (4.3%) | 16 (16.5%) |
| Events^{d} | | | |
| Maximum Severity of | | | |
| Potential Neurotoxicity | | | |
| Events^{d} | | | |
| Grade 1 | 2 (6.7%) | 1(4.3%) | 9 (9.3%) |
| Grade 2 | 3 (10.0%) | 0 | 7 (7.2%) |
| Grade 3 | 0 | 0 | 0 |
| Grade 4 | 0 | 0 | 0 |
| Grade 5 | 0 | 0 | 0 |
| Serious | 1(3.3%) | 0 | 3 (3.1%) |
| Neurotoxicity (related)^{e} | 2 (6.7%) | 0 | 5 (5.2%) |
| Grade 3 or higher | 0 | 0 | 0 |
| Infection-related TEAE | 11(36.7%) | 3 (13.0%) | 32 (33.0%) |
| Grade 3 or higher | 1(3.3%) | 1(4.3%) | 9(9.3%) |
| Drug-related | 6 (20.0%) | 0 | 11(11.3%) |
| Grade 3 or higher | 0 | 0 | 1(1.0%) |
| Infusion/Injection | | | |
| Reaction | | | |
| TEAE | 5 (16.7%) | 3 (13.0%) | 15 (15.5%) |
| Drug-related | 5 (16.7%) | 3 (13.0%) | 15 (15.5%) |
| Grade 1 or 2 | 5 (16.7%) | 3 (13.0%) | 15 (15.5%) |

Keys: TEAE = treatment-emergent adverse event,
CRS = cytokine release syndrome,.
^{a}Treatment discontinuation due to adverse event on the end of treatment CRF page.
^{b}CRS events are linked for the same subject after the same infusion. If one CRS event is followed by another with an onset date the same as or 1 day after the end date of the previous CRS and any features of the CRS (i.e.: toxicity grades/seriousness/action taken) are different between the CRS events, these CRS events are linked together and considered as one event.
^{c}Death due to adverse event on the adverse event CRF page.
^{d}The preferred terms identified as potential neurotoxicity events are Amnesia, Aphonia, Bradyphrenia, Confusional state, Delirium, Depressed level of consciousness, Disorientation, Dysarthria, Encephalopathy, Feeling abnormal, Hallucination, Lethargy, Memory impairment, Pyrexia, Somnolence, VIth nerve paralysis.
^{e}Neurotoxicity is defined as a potential neurotoxicity event that was considered related by investigator.
Percentages are calculated with the number of subjects in each group as denominator.
*Includes Part 2 subjects.

### Dose-Limiting Toxicity (DLT)

DLTs were evaluated in Part 1 (dose escalation) only. In Part 1, no subject who received talquetamab at the RP2D of 405 μg/kg SC weekly experienced a DLT, and 1 subject who received talquetamab at the RP2D of 800 ug/kg SC biweekly experienced a DLT (Table 13). No subjects in Part 2 experienced TEAEs meeting DLT criteria.

Three DLTs have been reported in subjects receiving any dose of SC talquetamab (Table 13). One subject reported a SAE of Grade 3 maculopapular rash (deemed very likely related to talquetamab) after receiving two 135 μg/kg weekly treatment doses of talquetamab SC. The SAE improved to Grade 1 as of the data cutoff. One subject experienced a Grade 3 maculopapular rash after receiving a single 800 μg/kg treatment dose (weekly schedule) of talquetamab SC that was considered possibly related. One subject experienced a Grade 3 rash after receiving a single 800 ug/kg treatment dose (biweekly schedule) that was considered very likely related. Both events resolved and subjects continued on treatment and remain on treatment as of the data cutoff.

### Grade 3 or Higher Treatment-Emergent Adverse Events

### Talquetamab RP2D of 405 Ug/Kg SC Weekly

Twenty-five (83.3%) subjects receiving talquetamab at RP2D of 405 μg/kg SC Weekly had a Grade 3 or higher TEAE. Grade 3 CRS and infection-related TEAE were reported for 1 subject each (3.3%) receiving the RP2D of 405 μg/kg SC Weekly. No subject had a Grade 3 or higher neurotoxicity event, or infusion/injection reaction. No subject had a fatal (Grade 5) TEAE (Table 13).

### Talquetamab RP2D of 800 Ug/Kg SC Biweekly

Seventeen (73.9%) subjects receiving talquetamab at RP2D of 800 μg/kg SC biweekly had a Grade 3 or higher TEAE. Grade 3 infection-related TEAE were reported for 1 subject (4.3%) receiving the RP2D of 800 μg/kg SC biweekly. No subject had a Grade 3 or higher CRS, neurotoxicity event, or systemic administration related reaction, or local injection site reaction. One subject had a Grade 5 TEAE (Table 13).

### All Talquetamab SC Cohorts

Seventy-nine (81.4%) subjects receiving any dose of SC talquetamab had a Grade 3 or higher TEAE. Grade 3 or higher CRS and infection-related TEAEs were reported for 1 (1.0%) subject and 9 (9.3%) subjects, respectively. No subject had a Grade 3 or higher neurotoxicity event, infusion/injection reaction (Table 13). One subject had a Grade 5 neuroendocrine carcinoma that the investigator did not consider related to study treatment and 1 subject had Grade 5 TEAE (Table 13).

### Serious Adverse Events

Talquetamab RP2D of 405 μg/Kg SC Weekly

Among the 30 subjects receiving talquetamab SC at the RP2D of 405 μg/kg SC Weekly, serious TEAEs were reported for 10 subjects (33.3%). The only event reported as serious in more than 1 subject was CRS (2 subjects, 6.7%).

Talquetamab RP2D of 800 μg/Kg SC Biweekly

Among the 23 subjects receiving talquetamab SC at the RP2D of 800 μg/kg SC biweekly, serious TEAEs were reported for 5 subjects (21.7%). The only event reported as serious in more than 1 subject was pyrexia (2 subjects, 8.7%).

### All Talquetamab SC Cohorts

Serious TEAEs were reported for 37 subjects (38.1%) receiving talquetamab SC (Table 13). Serious adverse events reported by more than 1 subject were CRS (6 subjects, 6.2%), pyrexia (5 subjects, 5.2%), hypercalcemia, febrile neutropenia, bone pain (3 subjects, 3.1%), influenza, urinary tract infection, somnolence (2 subjects, 2.1%). Thirteen (13.4%) subjects had a study drug-related serious adverse event; among them, CRS (7.3%) and pyrexia (3.7%) were reported by more than 1 subject.

### Deaths

### All Talquetamab SC Cohorts

Seven subjects receiving talquetamab SC died as of the data cutoff date: 3 subjects due to disease progression, 1 due to the TEAE of neuroendocrine carcinoma that was considered not related to study drug, and 3 subjects within 100 days of last dose without subsequent anticancer therapy due to unknown reasons.

### Cytokine Release Syndrome (CRS)

The mechanism of action of talquetamab is based on the binding and activation of T cells and the release of cytokines in the tumor environment, thus CRS is expected in patients receiving talquetamab and CRS is an important identified risk for talquetamab with mitigation strategies in place in all ongoing and planned clinical studies. To reduce the risk of CRS, subjects receive step-up doses of talquetamab, and premedications (glucocorticoid, antihistamine, and antipyretic) prior to each step-up dose and the first treatment dose of talquetamab per protocol.

Talquetamab RP2D of 405 μg/Kg SC Weekly

CRS was reported in 23 subjects (76.7%) receiving the RP2D of 405 μg/kg SC Weekly, mostly Grade 1 (60.0%) or Grade 2 (1033%) (Table 13). One patient (3.3%) experienced a Grade 3 CRS event; the patient recovered and continued treatment. CRS was only seen during early doses in Cycle 1, and the median duration of CRS was 2 days (range: 1 to 3 days). Twenty-two subjects (73.3%) received supportive measures as treatment for CRS (18 [60%] received tocilizumab, 1 subject [3.3%] each received corticosteroids, vasopressors, and supplemental oxygen).

Talquetamab RP2D of 800 μg/kg SC Biweekly

CRS was reported in 18 subjects (78.3%) receiving the RP2D of 800 μg/kg SC biweekly, all either Grade 1 (52.2%) or Grade 2 (26.1%) (Table 13). CRS was only seen during early doses in Cycle 1, and the median duration of CRS was 2 days (range: 1 to 5 days). Seventeen subjects (73.9%) received supportive measures as treatment for CRS (15 [65.2%] received tocilizumab, 1 subject [4.3%] each received corticosteroids, and supplemental oxygen).

### All Talquetamab SC Cohorts

CRS was reported in 70.1% of subjects receiving talquetamab SC, mostly Grade 1 (50.5%) or Grade 2 (18.6%) (Table 13). One subject (1.0%) exhibited a Grade 3 CRS event. The incidence of CRS and associated symptoms appeared to be dose dependent. CRS was only seen during early doses in Cycle 1, and the median duration of CRS was 2 days (range: 1 to 5 days). Sixty-five subjects (67.0%) received supportive measures as treatment for CRS (50 [51.5%] received tocilizumab, 4 subjects [4.1%] received steroids, 2 subjects [2.1%] received vasopressors, and 8 subjects [8.2%] received supplemental oxygen).

### Neurological Adverse Events

Based on the mode of action of talquetamab, neurotoxicity is identified as an important potential risk. In June 2018, members of the ASTCT developed a severity grading system for CRS and ICANS events induced by CAR-T cells and may be applied to other biologics. It was published in April of 2019. This study began in January 2018; consequently, TEAEs in Part 1 and Part 2 of this study were not coded using the ASTCT guidelines. To manage this retrospectively, potential neurotoxicity events (regardless of investigator-assessed relatedness) were identified by the applicant's medical team via review of TEAEs reported in the system organ classes of Nervous System Disorders and Psychiatric Disorders against a predetermined list. The subset of these events that were judged by the investigator as talquetamab-related were considered neurotoxicity events. TEAEs in Part 3 of this study and other ongoing or planned studies will be coded following the ASTCT guidelines and use the term ICANS for neurotoxicity reporting.

Talquetamab RP2D of 405 μg/Kg SC Weekly

Among the 30 subjects receiving talquetamab SC at the RP2D of 405 μg/kg SC Weekly, neurotoxicity events were reported in 2 (6.7%) subjects. Of the neurotoxicity events reported, none were reported by more the one subject.

Talquetamab RP2D of 800 μg/kg SC Biweekly

Among the 23 subjects receiving talquetamab SC at the RP2D of 405 μg/kg SC Weekly, there were no neurotoxicity events reported.

### All Talquetamab SC Cohorts

Among the 97 subjects receiving any dose of talquetamab SC as of the data cutoff, neurotoxicity events were reported in 5 (5.2%) subjects. Of the neurotoxicity events reported, none were reported by more the one subject.

### Pharmacokinetics

As of 25 Mar. 2021 (cutoff for PK data in this study), PK data are available from 69 subjects treated with SC talquetamab at doses ranging from 5 to 800 μg/kg weekly and 800 μg/kg biweekly from MonumenTAL-1. PK data are also available from 100 subjects treated with IV talquetamab at doses ranging from 0.5 to 3.38 μg/kg biweekly and 1.5 to 180 μg/kg weekly. Based on the safety, efficacy and PK, 405 μg/kg weekly SC administration was identified as the putative RP2D and being evaluated in the Part 2 and Part 3 (400 μg/kg for operational convenience) of MonumenTAL-1.

Following IV administration, preliminary results demonstrated that Cₘₐₓ occurred at the end of IV infusion. Talquetamab levels declined quickly with an elimination half-life (t_{1/2}) of approximately 7 days. Exposure increased in an approximately dose proportional manner following IV treatment across the range of 1.5 to 180 μg/kg.

The PK of talquetamab was further evaluated after talquetamab was subcutaneously administered weekly or biweekly. Following weekly SC administration, the concentration-time profiles demonstrated a less fluctuated and more sustained pattern. The preliminary results suggested the individual Tₘₐₓ occurred on Day 2 to Day 8. At the similar dose level of talquetamab, Cₘₐₓ was approximately 5.6-fold lower than that of IV treatment; talquetamab trough levels were comparable between IV and SC administration. The sponsor acknowledges that 400 μg/kg SC weekly will result in higher mean C_{trough}, lower mean Cₘₐₓ and similar mean C_{avg} at steady state compared to those at 800 μg/kg SC biweekly. However, the inter-subject variability in talquetamab PK was substantial, eg, the CV for most PK parameters were higher than 50%. Consequently, the C_{trough} and Cₘₐₓ at steady state overlapped substantially between 400 μg/kg SC weekly and 800 μg/kg SC biweekly. Both 400 μg/kg SC weekly and 800 μg/kg SC biweekly were identified as RP2D based on the observed efficacy and safety. Due to the unique and novel mechanism of action of talquetamab, it is not clear whether the Cₘₐₓ is the driving force for the efficacy. However, the talquetamab C_{trough} from both dose regimens was comparable or higher than the maximum effect (EC₉₀) values identified in an ex vivo cytotoxicity assay. In addition, at the dose levels equal to or higher than 405 μg/kg weekly and 800 μg/kg biweekly, the mean talquetamab concentrations were higher than the maximum EC₉₀ obtained from ex vivo cytotoxicity assay using bone marrow mononuclear cells from multiple myeloma patients (n=6). This assay assessed the ability of talquetamab to induce killing using mononuclear cells from the bone marrow samples of patients with multiple myeloma in co-culture with T cells from healthy donors. Based on the available steady-state data in Cycle 3, the mean accumulation ratio (based on AUCₜₐᵤ) following SC weekly dosing ranged from 1.7 to 5.1. Furthermore, preliminary population PK analysis and non-compartmental analysis showed that the mean bioavailability following SC weekly administration was 48%.

A summary of talquetamab PK parameters, as of 25 Mar. 2021, on Cycle 1 and Cycle 3 following multiple SC weekly (400 μg/kg) and biweekly (800 μg/kg) dosing is provided in Table 14.

TABLE 14
Preliminary Summary of Talquetamab PK Parameters on Cycle 1
and Cycle 3 Following Multiple SC Weekly Dosing (400 +82g/kg) and
Biweekly Dosing (800 μg/kg) of Talquetamab

| | Dose | | AUCₜₐᵤ | | |
|---|---|---|---|---|---|
| Cohort | Regimen | Cₘₐₓ [ng/mL] | [ng · h/mL]^{a} | AR_{Auc} | C_{trough} [ng/mL] |
| | Talquetamab PK parameters[mean(SD)] (Cycle 1) | | | | |
| Cohorts 20 and 23^{b} | 400 qlw | 1363 (992) | 139903 (106157) | | 876 (698) (N = 19) |
| (N = 25) | | | | | |
| Cohort 24 (N = 12) | 800 q2w | 2395 (1647) | 606614 (386533) | | 1203 (688) (N = 10) |
| | Talquetamab PK parameters [mean(SD)] (Cycle 3) | | | | |
| Cohorts 20 and 23^{b} | 400 qlw | 3243 (1567) | 499243 (241097) | 4.7(3.0) | 2805 (1460) |
| (N = 17) | | | | | (N = 14) |
| Cohort 24 (N = 8) | 800 q2w | 3901 (1408) | 1015793 (378659) | 2.4 (2.2) | 2211 (657) (N = 7) |

^{a}For qlw, AUCₜₐᵤ = AUC_{0-168 hr};
For q2w, AUCₜₐᵤ = AUC_{0-336 hr}
^{b}PK parameters from Cohorts 20 and 23 were combined

### Immunogenicity

As of 7 Apr. 7 2021 (cutoff for immunogenicity data in Study 64007957MMY1001), samples from 161 subjects (IV and SC treatment) were evaluated for the immunogenicity. Preliminary results showed 12 of 100 subjects (12%) for IV administration (up to 180 μg/kg) and 7 of 61 subjects (11%) for SC administration (up to 800 μg/kg) were positive for ADAs against talquetamab. The titers in majority of the subjects were low (equal to the minimum required dilution of the assay [20]), except for 2 subjects. Based on the preliminary evaluation, ADAs do not appear to impact the exposure of talquetamab.

As part of the initial scientific advice, CHMP highlighted the need to provide 1-year data on immunogenicity for marketing authorization. Immunogenicity data have already been obtained from this study (Parts 1 and 2) and will be collected from Part 3 (Phase 2). At the time of submission, 1-year data will be available from subjects in Part 1 and Part 2 who are still in the study after 1 year. In addition, approximately 6 months of immunogenicity data from subjects in Part 3 of this study will be included.

### Pharmacodynamics

As of data cutoff of 5 Feb. 2021, pharmacodynamic data are available for 28 participants treated at the RP2D in MomenTAL-1. Subjects who received 405 μg/kg SC talquetamab demonstrated pharmacodynamic changes consistent with the proposed mechanism of action. These included consistent increases in cytokines such as IL-10 (median maximum fold change 8.582; range: [1.42-73.82]), IL-2Rα (3.866; 1.47-27.84), and IL-6 (87.800; 1.45-1841.25). In addition, induction of T cell activation, as evidenced by increased expression of activation markers on CD3+ T cells such as CD25 (median maximum fold change 1.87 [range: 0.72 to 9.76]), PD-1 (1.94; 1.09-6.51), HLA-DR (1.324; 0.76-5.64), CD38 (2.952; 0.6-11.30), LAG-3 (3.221; 1.16-11.36), TIM-3 (3.442; 1.06-15.09) and T cell redistribution as indicated by changes in total T cell absolute counts (0.623; 0.2-4.18) were also observed in the 405 μg/kg cohort.

### Conclusions Updated Data Cutoff

As of Jul. 19, 2021, 95 patients have received SC talquetamab. The RP2D was originally identified as a weekly SC dose of 405 μg/kg talquetamab with step-up doses of 10 and 60 μg/kg. Alternative dosing schedules that require less frequent administration continue to be investigated. A biweekly RP2D was also identified as a SC dose of 800 μg/kg talquetamab with step-up doses of 10, 60, and 300 μg/kg.

30 patients received the 405 μg/kg weekly dosing schedule (median age: 61.5 years [range: 46-80]; 63% male; 100% triple-class exposed; 80% penta-drug exposed; 77% triple-class refractory, 20% penta-drug refractory; 30% prior BCMA-directed therapy). 23 patients received the 800 μg/kg biweekly dosing schedule (median age: 60.0 years [range: 47-84]; 52% female; 96% triple-class exposed; 70% penta-drug exposed; 65% triple-class refractory, 22% penta-drug refractory; 17% prior BCMA-directed therapy).

There were no treatment discontinuations due to AEs at either of the RP2Ds. The most common AEs at the 405 μg/kg weekly dose were CRS (73%; grade 3/4: 3%), neutropenia (67%; grade 3/4: 60%), and dysgeusia (60%; all grade 1/2); skin-related AEs occurred in 77% (nail disorders: 30%) of patients, and infections occurred in 37% of patients (grade 3/4: 3%). The most common AEs at the 800 μg/kg biweekly dose were CRS (78%; all grade 1/2), dry mouth (44%; all grade 1/2), and neutropenia (44%; grade 3/4: 35%); skin-related AEs occurred in 65% of patients (nail disorders: 17%) and infections occurred in 13% of patients (grade 3/4: 4%).

At a median follow-up of 7.5 months (range: 0.9-15.2), the overall response rate (ORR) was 70% (very good partial response or better [≥VGPR]: 57%) in 30 response-evaluable patients treated with the 405 μg/kg weekly dose. At a median follow-up of 3.7 months (range 0.0-12.0), the ORR was 71% (≥VGPR: 53%) in the 17 response-evaluable patients who received 800 μg/kg biweekly doses. Responses were durable and deepened over time in both cohorts (Figure); Median duration of response was not reached. The majority of responses were maintained at 6 months, with 66% (10/15) and 85% (7/8) of patients continuing on treatment in the 405 μg/kg weekly and 800 μg/kg biweekly cohorts, respectively. Serum trough levels of talquetamab were comparable at both RP2Ds. Pharmacodynamic data from cohorts treated at both dose levels showed consistent peripheral T cell activation and induction of cytokines, demonstrating the mechanism of action for talquetamab.

These findings indicate that SC talquetamab is well tolerated and highly effective at both RP2Ds. Preliminary data from the 800 μg/kg biweekly cohort indicate that less frequent, higher doses of SC talquetamab do not have a negative impact on the previously described safety profile.

### Pharmacokinetics Simulation

Further data simulation provided pharmacokinetics (PK) data support for the RP2D of 800 μg/kg administered at cycles 1 and 2. Based on the simulation results, the maximum mean serum concentration (Cₘₐₓ) for patients treated with cycle 2 of 800 g/kg was 4233.4 ng/mL (range 1247.4-12710.1 ng/mL) and the lowest mean concentration of talquetamab in the blood (Cₘᵢₙ) was 2670.4 ng/mL (range 662.7-8100.4 ng/mL). A summary of the simulated exposure metrics (Cₘₐₓ, Cₘᵢₙ, and AUC (area under the curve)) for 4 different doses of talquetamab (including 800 μg/kg) at cycle 2 are shown in Table 15.

TABLE 15
Summary of the simulated exposure metrics mean Cmax, Cmin, and AUC
across different dosing regiments of talquetamab at cycle 2.

| | | | AUC, ng × day/mL | |
|---|---|---|---|---|
| Dose | | | [over a dosing | |
| Regimen | Cmax, ng/mL | Cmin, ng/mL | interval] | AUC₀₋₁₄ |
| 800 | 4233.4 (1247.4- | 2670.4 (662.7- | 25574.8 (7349.3- | 51149.6 |
| Q1Wa | 12710.1) | 8100.4) | 77736.2) | |
| 400 | 2116.7 (623.7- | 1335.2 (331.3- | 12787.4 (3674.6- | 25574.8 |
| Q1Wa | 6355.1) | 4050.2) | 38868.1) | |
| 800 | 2469.4 (772.9- | 1094.0 (178.6- | 26277.8 (7562.2- | 26277.8 |
| Q2Wa | 6894.6) | 3711.2) | 80807.8) | |
| 1200 | 3704.1 (1159.3- | 1641.1 (268.0- | 39416.7 (11343.3- | 39416.7 |
| Q2Wa | 10341.9) | 5566.9) | 121211.8) | |
| 1600 | 4938.8 (1545.7- | 2188.1 (357.3- | 52555.6 (15124.4- | 52555.6 |
| Q2Wa | 13789.2) | 7422.5) | 161615.7) | |
| 1600 | 3606.3 (1188.8- | 994.0 (127.5- | 50509.1 (14585.4- | 33672.7 |
| Q3wb | 9222.1) | 3607.2) | 149449.3) | |
| 1600 | 3403.7 (1099.1- | 703.8 (60.5- | 54714.5 (15290.0- | 27357.3 |
| Q4Wa | 8626.6) | 2982.0) | 171443.3) | |

Median (5^{th} and 95^{th} percentiles) of prediction
^{a}Exposure metrics calculated for 2^{nd} Cycle
^{b}Exposure metrics calculated after 2^{nd} dose

The mean Cₘₐₓ for patients treated with 800 μg/kg of talquetamab at a steady state was 4808.9 ng/mL (range 1329.0-18938.7 ng/mL) and the mean Cₘᵢₙ was 3908.3 ng/mL (range 751.2-16766.7 ng/mL). A summary of the simulated exposure metrics (Cₘₐₓ, Cₘᵢₙ, and AUC) across different dosing regiments of talquetamab (including 800 μg/kg) at a steady state are shown in Table 16.

TABLE 16
Summary of exposure metrics mean Cₘₐₓ, Cₘᵢₙ, and AUC across different
dosing regimens of talquetamab at a steady state.

| | | | AUC, ng × day/mL | |
|---|---|---|---|---|
| Dose | | | [over a dosing | |
| Regimen | Cmax, ng/mL | Cmin, ng/mL | interval] | AUC₀₋₁₄ |
| 800 | 4808.9 (1329.0- | 3908.3 (751.2- | 31419.9 (7783.8- | 62839.8 |
| Q1W | 18938.7) | 16766.7) | 127114.7) | |
| 400 | 2404.4 (664.5- | 1954.2 (375.6- | 15709.9 (3891.9- | 31419.8 |
| Q1W | 9469.4) | 8383.4) | 63557.3) | |
| 800 | 2816.6 (828.6- | 1578.7 (192.6- | 31426.7 (7783.8- | 31426.7 |
| Q2W | 9947.7) | 7730.7) | 127687.7) | |
| 1200 | 4224.9 (1242.9- | 2368.1 (289.0- | 47140.0 (11675.7- | 47140.0 |
| Q2W | 14921.5) | 11596.0) | 191531.6) | |
| 1600 | 5633.4 (1657.2- | 3157.4(385.3- | 62853.4 (15567.6- | 62853.4 |
| Q2W | 19895.4) | 15461.4) | 255375.4) | |
| 1600 | 4498.9 (1345.3- | 1686.1 (152.3- | 62854.1 (15567.6- | 41902.2 |
| Q3W | 14159.5) | 9719.1) | 255468.4) | |
| 1600 | 3940.9 (1167.6- | 1040.8 (64.9- | 62858.8 (15567.6- | 31429.4 |
| Q4W | 11640.4) | 6866.8) | 257793.1) | |

Median (5^{th} and 95^{th} percentiles) of prediction

Part 2 (Dose Expansion Part)—Dosing Schedule

Subjects are treated in Part 2 (NCT04634552) after a putative RP2D(s) for talquetamab was identified in Part 1.

SC administration: Upon identification of the putative RP2D for SC administration, subjects were treated with 405 μg/kg as the SC QW RP2D in dose expansion (with step-up doses of 10 and 60 μg/kg) to further demonstrate safety and to characterize preliminary antitumor activity. Another subset of subjects were treated with 800 μg/kg as the SC biweekly RP2D dose in dose expansion (with step-up doses of 10, 60, and 300 μg/kg).

In Part 2, up to 40 subjects can be enrolled and treated with IV or SC talquetamab at an RP2D of 405 μg/kg or 800 μg/kg. Additionally, the same dosing schedules recommended in Part 1 may be used to further characterize preliminary antitumor activity and safety in additional subjects at the RP2D doses of interest. The same supportive care measures used in Part 1 of the study will be applied to the subjected treated in Part 2. The SET (Study Evaluation Team) may stop further enrollment into the dose expansion cohorts pending the outcome of any SET reviews, or if treatment-emergent toxicity is determined to result in an unfavorable change in subject risk or benefit.

### Stopping Rule

For reach route of administration in Part 2, dose-limiting toxicity (DLT) data will be evaluated after approximately every 5 subjects (e.g., 5^{th}, 10^{th}, etc.) at a putative RP2D complete Cycle 1 of treatment or discontinue earlier. A BLRM model will be fitted, including all cumulative data for subjects from Part 1 (dose escalation) and Part 2 (dose expansion). If the posterior probability that the DLT rate is in the range of [0.25-1] is great than or equal to 25% (i.e., Prob[p)TOX, putative RP2D)>0.25 data]≥0.25), the sponsor will stop additional enrollment for the specific putative RP2D cohort. All DLTs that have occurred during Cycle 1 or Cycle 2 up to the current time point will be considered when fitting the BLRM model to calculate the posterior probability. If enrollment to the current putative RP2D is stopped based on SET reviews, other expansion cohorts at lower dose levels may be explored.

Part 3 (Phase 2, Study Name: MonumenTAL-1)

In Part 3, three different cohorts will be enrolled, Cohort A, Cohort B, and Cohort C. Cohorts A, B, and C are representative of the subset of relapsed/refractory multiple myeloma patients who have limited treatment options. These cohorts are defined as follows:

Cohort A (400 μg/kg weekly SC) will enroll subjects with multiple myeloma who have previously received ≥3 prior lines of therapy that included at least one proteasome inhibitor (PI), one immunomodulatory imide drug (IMiD), and an anti-CD38 monoclonal antibody, and have not been exposed to T cell redirection therapies such as CAR-T or bispecific antibodies.

Cohort B (400 μg/kg weekly SC) will enroll subjects with multiple myeloma who have previously received ≥3 prior lines of therapy that included at least one PI, one IMiD, and an anti-CD38 monoclonal antibody, and have been exposed to T cell redirection therapies such as CAR-T or bispecific antibodies.

Cohort C (800 μg/kg bi-weekly SC) will enroll subjects with multiple myeloma who have previously received ≥3 prior lines of therapy that included at least one PI, one IMiD, and an anti-CD38 monoclonal antibody, and have not been exposed to T cell redirection therapies such as CAR-T or bispecific antibodies.

Cohorts A and B will be enrolled after approximately 20 subjects have been treated with SC talquetamab at the RP2D of 400 μg/kg or 800 μg/kg for at least one cycle. The sponsor may also determine that additional subjects are required to further evaluate safety and dose prior to proceeding to Part 3. Enrollment for Cohort C will begin after 20 subjects have been treated with SC talquetamab at an RP2D of 800 μg/kg bi-weekly for at least 1 cycle. In contrast to Part 2, the selected RP2Ds for Part 3 are 400 μg/kg weekly and 800 μg/kg biweekly SC talquetamab until progressive disease. The putative RP2Ds in Part 2 are 405 μg/kg weekly and 800 μg/kg biweekly SC talquetamab until progressive disease.

In Part 3, subjects will be allowed to switch from a weekly 400 μg/kg SC dosing schedule to a biweekly 800 μg/kg SC dosing when a subject exhibits a complete response (CR) or better for a minimum of 6 months. The change in dosing schedule also must be approved by the sponsor, based on comparable preliminary PK, safety, and efficacy data of these 2 dosing schedules. The priming dose schedule in Part 3 will consist of 2 doses (10 and 60 μg/kg), each separated by 2 to 4 days and will be completed 2 to 4 days prior to the first treatment dose (i.e., if there are no delays in treatment, the first priming dose (10 μg/kg) is to be administered 5 to 8 days before the first treatment dose and the second priming dose (60 μg/kg 2 to 4 days before the first treatment dose). The biomarkers of the patients in Part 3 also will be evaluated.

An adverse event of special interest for Part 3 is a neurotoxicity grade ≥2 (i.e., ICANS (immune effector cell-associated neurotoxicity syndrome), symptoms of ICANS, and non-ICANS neurotoxicity). Regarding the T&E neurological examination for Part 3, an assessment occurs at priming dose 1 and there is no assessment at screening.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

What is claimed:

1. A method of treating a hematological malignancy, preferably a multiple myeloma, in a subject in need thereof, comprising administering to the subject a therapeutically effective amount of a GPRC5D×CD3 bispecific antibody or antigen binding fragment thereof, wherein the subject is relapsed or refractory to treatment with a prior anti-cancer treatment.

2. The method of claim 1, wherein the GPRC5D×CD3 bispecific antibody is administered intravenously or subcutaneously at a dose of about 0.2 μg/kg to about 2400 μg/kg.

3. The method of claim 2, comprising:
(1) administering to the subject the GPRC5D×CD3 bispecific antibody intravenously or subcutaneously at a first priming dose of 0.5 μg/kg to 10 μg/kg, such as 0.5 μg/kg, 1.0 μg/kg, 1.5 μg/kg, 2.25 μg/kg, 2.5 μg/kg, 2.75 μg/kg, 3.0 μg/kg, 3.25 μg/kg, 3.38 μg/kg, 3.5 μg/kg, 3.75 μg/kg, 4 μg/kg, 4.5 μg/kg, 5 μg/kg, 6 μg/kg, 7 μg/kg, 8 μg/kg, 9 μg/kg, 10 μg/kg, or any value in between, preferably every 2 to 4 days, and
(2) subsequently administering to the subject the GPRC5D×CD3 bispecific antibody subcutaneously at a treatment dose higher than the first priming dose, in the range of 1.5 μg/kg to 2400 μg/kg or 1.5 μg/kg to 1000 μg/kg, such as 1.5 μg/kg, 5 μg/kg, 10 μg/kg, 15 μg/kg, 30 μg/kg, 45 μg/kg, 100 μg/kg, 135 μg/kg, 200 μg/kg, 300 μg/kg, 400 μg/kg, 405 μg/kg, 500 μg/kg, 600 μg/kg, 700 μg/kg, 800 μg/kg, 900 μg/kg, 1000 μg/kg, 1200 μg/kg, 1600 μg/kg, 2000 μg/kg, 2400 μg/kg, or any value in between, preferably monthly, tri-weekly, bi-weekly, weekly or twice a week,
optionally, the method further comprising administering to the subject the GPRC5D×CD3 bispecific antibody subcutaneously at one or more additional priming doses higher than the first priming dose but lower than the treatment dose, wherein the one or more additional priming doses are administered after the first priming dose but before the administration of the treatment dose.

4. The method of claim 2, wherein the GPRC5D×CD3 bispecific antibody is administered intravenously at a dose of about 0.2 μg/kg to about 500 μg/kg, preferably about 1 μg/kg to about 300 μg/kg, most preferably about 10 μg/kg to about 200 μg/kg, such as about 10, 20, 30, 40, 50, 60, 70, 80, 90, 100, 110, 120, 130, 140, 150, 160, 170, 180, 190, 200 μg/kg, or any value in between, preferably monthly, tri-weekly, bi-weekly, weekly, or twice a week.

5. The method of claim 4, comprising:
(1) administering to the subject the GPRC5D×CD3 bispecific antibody intravenously at a first priming dose of 0.5 μg/kg to 5 μg/kg, such as 0.5 μg/kg, 1.0 μg/kg, 1.5 μg/kg, 2.25 μg/kg, 2.5 μg/kg, 2.75 μg/kg, 3.0 μg/kg, 3.25 μg/kg, 3.38 μg/kg, 3.5 μg/kg, 3.75 μg/kg, 4 μg/kg, 4.5 μg/kg, 5 μg/kg, or any value in between preferably every 2 to 4 days, and
(2) subsequently administering to the subject the GPRC5D×CD3 bispecific antibody intravenously at a treatment dose higher than the first priming dose, in the range of 1.5 μg/kg to 200 μg/kg, such as 1.5 μg/kg, 2.25 μg/kg, 3.38 μg/kg, 5 μg/kg, 7.5 μg/kg, 11.25 μg/kg, 20 μg/kg, 40 μg/kg, 60 μg/kg, 80 μg/kg, 100 μg/kg, 120 μg/kg, 140 μg/kg, 160 μg/kg, 180 μg/kg, 200 μg/kg, or any value in between, preferably monthly, tri-weekly, bi-weekly, weekly or twice a week,
optionally, the method further comprising administering to the subject the GPRC5D×CD3 bispecific antibody intravenously at one or more additional priming doses higher than the first priming dose but lower than the treatment dose, wherein the one or more additional priming doses are administered after the first priming dose but before the administration of the treatment dose.

6. The method of claim 2, wherein the GPRC5D×CD3 bispecific antibody is administered subcutaneously at a dose of about 0.5 μg/kg to about 2400 μg/kg, or about 1 μg/kg to about 2400 μg/kg, or about 10 μg/kg to about 2400 μg/kg, such as about 10, 50, 100, 150, 200, 250, 300, 350, 400, 450, 500, 550, 600, 650, 700, 750, 800, 850, 900, 950, 1000, 1200, 1600, 2000, 2400 μg/kg, or any value in between, preferably monthly, tri-weekly, bi-weekly, weekly, or twice a week.

7. The method of claim 6, comprising:
(1) administering to the subject the GPRC5D×CD3 bispecific antibody at one or more priming doses of 0.3 μg/kg to 400 μg/kg, preferably every 2 to 4 days, and
(2) subsequently administering to the subject the GPRC5D×CD3 bispecific antibody at a treatment dose higher than the priming doses, such as 1 μg/kg to 2400 μg/kg, preferably monthly, tri-weekly, bi-weekly, weekly or twice a week.

8. A method of treating a multiple myeloma in a subject in need thereof, comprising subcutaneously administering to the subject 405 μg/kg of a GPRC5D×CD3 bispecific antibody or antigen binding fragment thereof weekly or biweekly, wherein the subject is relapsed or refractory to treatment with a prior anti-cancer treatment, preferably the initial administration of the 405 μg/kg GPRC5D×CD3 bispecific antibody or antigen binding fragment thereof occurs after the subject is administered with one or more priming doses of the antibody or antigen binding fragment thereof, such as one or more priming doses of 10, 60, and 300 μg/kg administered subcutaneously weekly or biweekly.

9. A method of treating a multiple myeloma in a subject in need thereof, comprising subcutaneously administering to the subject 800 μg/kg of a GPRC5D×CD3 bispecific antibody or antigen binding fragment thereof weekly or biweekly, wherein the subject is relapsed or refractory to treatment with a prior anti-cancer treatment, preferably the initial administration of the 800 μg/kg GPRC5D×CD3 bispecific antibody or antigen binding fragment thereof occurs after the subject is administered with one or more priming doses of the antibody or antigen binding fragment thereof, such as one or more priming doses of 10, 60, and 300 μg/kg administered subcutaneously weekly or biweekly.

10. The method of any one of claims 1 to 9, wherein the GPRC5D×CD3 bispecific antibody or antigen binding fragment thereof comprises a GPRC5D binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

11. The method of any one of claims 1 to 10, wherein the GPRC5D binding domain comprises a heavy chain variable region (VH) having the amino acid sequence of SEQ ID NO: 10 and a light chain variable region (VL) having the amino acid sequence of SEQ ID NO: 11, and the CD3 biding domain comprises a VH having the amino acid sequence of SEQ ID NO: 20 and a VL having the amino acid sequence of SEQ ID NO: 21.

12. The method of any one of claims 1 to 11, wherein the GPRC5D×CD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

13. The method of any one of claims 1 to 12, wherein the GPRC5D×CD3 bispecific antibody further comprises proline at position 228, alanine at position 234 and alanine at position 235 in both the HC1 and the HC2.

14. The method of any one of claims 1 to 13, wherein the GPRC5D×CD3 bispecific antibody comprises the HC1 having the amino acid sequence of SEQ ID NO: 12, a first light chain (LC1) having the amino acid sequence of SEQ ID NO: 13, the HC2 having the amino acid sequence of SEQ ID NO: 22 and a second light chain (LC2) having the amino acid sequence of SEQ ID NO: 23.

15. The method of any one of claims 1 to 14, wherein the GPRC5D×CD3 bispecific antibody is talquetamab.

16. The method of any one of claims 1 to 15, wherein the treatment achieves a complete response, stringent complete response, very good partial response, partial response, minimal response or stable disease status, and can be continued until disease progression or lack of patient benefit.

17. The method of claim 16, wherein the treatment achieves the complete response that is characterized by negative minimal residual disease (MRD) status, preferably negative MRD status at 10⁻⁶ cells, as determined by next generation sequencing (NGS), or an overall response rate of at least 20%, such as at least 20%, 25%, 30%, 35%, 40%, 45%, 50%, 55%, 60%, 65%, 70%, 75%, 80%, 85%, 90%, 95%, 100%, or any value in between.

18. The method of any one of claims 1 to 17, wherein the treatment results in an exposure of GPRC5D×CD3 bispecific antibody at a steady state mean Cmax of 10 to 25,000 ng/ml, such as 100 to 20,000 ng/ml or 1000-10,000 ng/ml, and a steady state mean AUC_{0-14d} of 1000 to 1,500,000 ng h/ml, such as 5000 to 1,000,000 ng h/ml or 10,000 to 1,000,000 ng h/mL.

19. The method of any one of claims 1 to 18, wherein the prior anti-cancer treatment is selected from the group consisting of thalidomide, lenalidomide, pomalidomide, bortezomib, ixazomib, carfilzomib, panobinostat, pamidronate, zoledronic acid, daratumumab, elotuzumab, melphalan, selinexor, belantamab mafodotin-blmf, Venetoclax, CC-92480, CAR-T therapies, other BCMA-directed therapies, other CD38-directed therapies, and combinations of two or more thereof.

20. The method of any one of claims 1 to 19, wherein the subject is a human in need of a treatment of multiple myeloma and is relapsed or refractory to treatment with the prior treatment of the multiple myeloma.

21. The method of any one of claims 1 to 20, further comprising administering to the subject one or more additional anti-cancer therapies.

22. The method of claim 21, wherein the one or more additional anti-cancer therapies are selected from the group consisting of autologous stem cell transplants (ASCT), radiation, surgery, chemotherapeutic agents, CAR-T therapies, cellular therapies, immunomodulatory agents, targeted cancer therapies, a therapy that reduces or depletes Treg, and combinations of two or more thereof.

23. The method of claim 21, wherein the one or more additional anti-cancer therapies are selected from the group consisting of selinexor, belantamab mafodotin-blmf, isatuximab, venetoclax, lenalidomide, thalidomide, pomalidomide, bortezomib, carfilzomib, elotozumab, ixazomib, melphalan, dexamethasone, vincristine, cyclophosphamide, hydroxydaunorubicin, prednisone, rituximab, imatinib, dasatinib, CC-92480, nilotinib, bosutinib, ponatinib, bafetinib, saracatinib, tozasertib, danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, and combinations of two or more thereof.
